(19) United States
(12) Patent Application Publication
(10) Pub. No.: US 2005/0250093 A1
(43) Pub. Date: Nov. 10, 2005

(54) Hepatitis c virus sub-genomic replicons

(76) Inventors: Adam T. Gates, Royersford, PA (US); Baohua Gu, Swedesford Road, MA (US); Robert T. Sarisky, Lansdale, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/509,921

(22) PCT Filed: Apr. 3, 2003
(86) PCT No.: PCT/US03/10177

§ 371 Date: Jun. 22, 2005

Related U.S. Application Data
(60) Provisional application No. 60/369,685, filed on Apr. 3, 2002.

(51) Int. Cl. C12Q 1/70; C12N 7/00; C12N 15/86

(52) U.S. Cl. 435/5; 435/235.1; 435/456; 435/325

(57) ABSTRACT

The present invention relates generally to the construction of sub-genomic HCV replicon systems that may provide the foundation for generating HCV replicons of all six major genotypes and subtypes to facilitate screening, testing, and evaluating anti-infective agents for HCV disease(s).

### TECHNICAL FIELD OF THE INVENTION

The present invention relates generally to the construction of sub-genomic HCV replicon systems that may provide the foundation for generating HCV replicons of all six major genotypes and subtypes to facilitate screening, testing, and evaluating anti-infective agents for HCV disease(s).

### BACKGROUND OF THE INVENTION

In the U.S., an estimated 4.5 million Americans are chronically infected with hepatitis C virus (HCV). Although only 30% of acute infections are symptomatic, greater than 85% of infected individuals develop chronic, persistent infection. Treatment costs for HCV infection have been estimated at $5.46 billion for the U.S. in 1997. Worldwide, over 200 million people are estimated to be infected chronically. HCV infection is responsible for 40-60% of all chronic liver disease and 30% of all liver transplants. The CDC estimates that the number of deaths due to HCV will minimally increase to 38,000/yr. by the year 2010.

Due to the high degree of variability in the viral surface antigens, existence of multiple viral genotypes, and demonstrated specificity of immunity, the development of a successful vaccine in the near future is unlikely. Alpha-interferon, alone or in combination with ribavirin, has been widely used since its approval for treatment of chronic HCV infection. However, adverse side effects are commonly associated with this treatment: flu-like symptoms, leukopenia, thrombocytopenia, and depression from interferon, as well as hemolytic anemia induced by ribavirin (Lindsay, 1997). This therapy remains less effective against infections caused by HCV genotype 1, which constitutes ˜75% of all HCV infections in the developed markets, compared to infections caused by the other five major HCV genotypes. Unfortunately, only ˜50-80% of patients respond to this treatment, measured by a reduction in serum HCV RNA levels and normalization of liver enzymes. Of those patients treated, 50-70% relapse within six months of cessation of treatment. Recently with the introduction of pegylated interferon, both initial and sustained response rates have improved substantially, and combination treatment of Peg-EFN with ribavirin constitutes the gold standard for therapy. However, side effects associated with the combination therapy and the impaired response in patients with genotype 1 present opportunities for improvement in disease management.

First identified by molecular cloning in 1989 (Choo et al, 1989), HCV is now widely accepted as the most common causative agent of post-transfusion non-A, non-B hepatitis (NANBH) (Kuo et al., 1989). Due to HCV's genome structure and sequence homology, this virus was assigned as a new genus in the Flaviviridae family. Like the other members of the Flaviviridae, such as flaviviruses (e.g., yellow fever virus and Dengue virus types 14) and pestiviruses (e.g., bovine viral diarrhea virus, border disease virus, and classic swine fever virus (Choo et al., 1989; Miller and Purcell, 1990)), HCV is an enveloped virus containing a single strand RNA molecule of positive polarity. The HCV genome is approximately 9.6 kilobases (kb) with a long, highly conserved, non-capped 5′ non-translated region (NTR) of approximately 340 bases which functions as an internal ribosome entry site (IRES) (Wang and Siddiqui, 1995). This element is followed by a region which encodes a single long open reading frame (ORF) encoding a polypeptide of ˜3000 amino acids comprising both the structural and nonstructural viral proteins.

Upon entry of the RNA into the cytoplasm of the cell, it is directly translated into a polypeptide of ˜3000 amino acids comprising both the structural and nonstructural viral proteins. This large polypeptide is subsequently processed into the individual structural and nonstructural proteins by a combination of host and virally-encoded proteinases (Kolykhalov et al., 1996). Following the termination codon at the end of the long ORF, there is a 3′ NTR which roughly consists of three regions: an ˜40 base region which is poorly conserved among various genotypes, a variable length poly (U)/polypyrimidine tract, and a highly conserved 98 base element also called the “3′ X-tail” (Kolykhalov et al, 1996; Tanaka et al, 1995; Tanaka et al, 1996; Yamada et al., 1996). The 3′ NTR is predicted to form a stable secondary structure that is essential for HCV growth in chimps and is believed to function in the initiation and regulation of viral RNA replication.

To study the biology of HCV and to screen for compounds that inhibit the virus replication, a cell-based system to grow the virus is essential. Recently, cell-based replicon systems for HCV were developed, in which the nonstructural proteins stably replicate sub genomic viral RNA in Huh-7 cells (Lohmann et al., Science 285:110 (1999) and Blight et al., Science 290:1972 (2000)). In the absence of a purified, functional HCV replicase consisting of viral non-structural and host proteins, our understanding of Flaviviridae RNA synthesis comes from studies using active recombinant RNA-dependent RNA polymerase (RdRp) and validation of these studies in the HCV replicon system. Novel chemical entities, which interfere with HCV RNA synthesis, are identified by screening compound banks for inhibition of recombinant HCV polymerase in biochemical assays. However, biochemical inhibition against this purified enzyme does not necessarily translate into replicon cell-based inhibition since in the latter system the polymerase exists within a replicase complex, associated with other viral and cellular polypeptides in appropriate stoichiometry. Moreover, in the absence of a robust small animal model for HCV infection, the replicon system more accurately represents an active infection than other in vitro systems.

However, despite the existence of infectious cDNA clones and many attempts to cultivate the virus in established cell lines in laboratories, efficient in vitro replication of the HCV virus has not been established prior to the present invention. A sub-genomic HCV replication system was successful in the human hepatoma cell line Huh-7 after electroporation of a particular genotype 1b, BB7 strain, RNA replicon containing a neo resistance marker (Lohmann et al., Science 285:110, 1999). The replicon is a di-cistronic construct containing the 5′ untranslated region of HCV (5′ NTR), a neomycin resistance gene (neo), the non-structural genes NS3 to NS5 and the 3′ non-translated region of HCV (3′ NTR). Translation of neo and NS3 to NS5 genes is mediated by HCV 5′ NTR and EMCV IRES, respectively. Improved replicons, containing high-levels of viral sub-genoric RNA copies were obtained by selection for adapted mutations in Huh-7 cells (Blight, K. J. et al. 2000; Lohmann et al. 2001; Guo et al. 2001).

Huh-7 cell-based replicon systems for HCV were constructed so that the non-structural proteins replicate sub-genomic viral RNA. One significant limitation of the available replicon systems is the inability of other genotypic derivatives, beyond that of two specific strains of genotype 1b (HCV-N and HCV-BB7), to replicate in Huh-7 cells. The generation of functional replicons for HCV genotypes 1 to 6 would be invaluable in efficiently developing antiviral agents, and solves a longstanding problem.

### SUMMARY OF THE INVENTION

In view of the aforementioned deficiencies associated with prior art HCV replicons, cell culture systems for the analysis of HCV replication, and the development of therapeutic compositions therefor, it is evident that there exists a need in the art for identification of chimeric sub-genomic replicons of HCV which can be incorporated into a cell capable of replicating RNA transcripts. These transcripts can then be used as target sequences for the production of attenuated HCV for vaccines, and targets for therapeutic compositions.

In accordance with the present invention, nucleotide sequences derived from various functional chimeric HCV replicons are provided herein. Through molecular cloning and tissue culture technologies as well as detailed analysis of the literature, the present invention describes the successful generation of stable cell lines expressing and replicating functional replicons, containing sequences from HCV genotype 1a (strain H77) or genotype 1b (strain J4) within the prototype 1b replicon backbone from HCV strain BB7. The methodology used to create these functional replicons described herein which express (1) genotype 1a sequences or (2) genotype 1b sequences from other strains not previously shown to be functional in such systems provides the foundation and know-how for generating HCV replicons of all six major genotypes and subtypes to facilitate screening, testing, and evaluating anti-infective agents for HCV disease(s).

One embodiment of the invention is a sub-genomic viral replicon that contains a nucleic acid construct encoding chimeric HCV nonstructural proteins and a complete NS5B polymerase. A further embodiment provides an NS5B encoding sequence linked in cis to a 3′UTR sequence of an HCV strain, preferably the same HCV strain.

In another embodiment, a chimeric replicon includes the nonstructural proteins NS3, NS4A, NS4B, NS5A, and NS5B. In a further embodiment of the invention, a chimeric replicon comprises an NS3 nucleotide sequence that encodes about the first 75 contiguous N-terminal amino acids of NS3 of genotype 1b. A preferred embodiment of the invention is a chimeric replicon that contains the NS3 encoding nucleotide sequence of a genotype 1b, BB7 strain. The nucleotide sequence that encodes the first 75 contiguous N-terminal amino acids of HCV type 1b, strain BB7 is

| | |
|---|---|
| ATGGCGCCTATTACGGCCTACTCCCAACAGACGCGAGGCCTACTTGGCTGCATCATCACTAGCCTCAC | (SEQ ID NO: 1) |
| AGGCCGGGACAGGAACCAGGTCGAGGGGGAGGTCCAAGTGGTCTCCACCGCAACACAATCTTTCCTGG | |
| CGACCTGCGTCAATGGCGTGTGTTGGACTGTCTATCATGGTGCCGGCTCAAAGACCCTTGCCGGCCCA | |
| AAGGGCCCAATCACCCAAATG | |

In still another embodiment of the invention, a chimeric replicon comprises an NS3 from any of the other six major HCV genotypes and subtypes wherein a particular NS3 has its N-terminal first 225 nucleotides replaced by the N-terminal first 225 nucleotides of NS3 of the genotype 1b, BB7 strain. For example, a preferred embodiment of the invention comprises a replicon wherein the N-terminal sequence of NS3 genotype 1b, BB7 strain, replaces the corresponding N-terminal sequence of NS3, genotype 1a. In addition, another embodiment is provided wherein a NS3 sequence is from the genotype 1a, H77 strain. In yet a further embodiment, an NS3 sequence is from the genotype 1b, J4 strain.

Another embodiment of the invention is a sub-genomic viral replicon that comprises a nucleic acid construct encoding chimeric HCV nonstructural proteins, and at least the C-terminal end of a strain specific NS5B polymerase gene linked in cis to a 3′UTR sequence from said strain. Specifically, a preferred embodiment of the invention is a chimeric replicon wherein the NS5B comprises sequence from both a BB7 strain and a J4 strain, see FIG. 2(b), the J4M/S construct. The C-terminal portion of BB7's NS5B is linked in cis to a 3′UTR sequence of a BB7 strain. However, the N-terminal portion of NS5B is sequence from the J4 strain. In an embodiment of the invention, there is provided a construct wherein less than the complete NS5B from a single HCV strain is used because both BB7 and J4 are of the same subtype, 1b.

In another embodiment of the invention, a replicating HCV sub-genomic replicon comprises SEQ ID NO:2, SEQ ID NO:6, SEQ ID NO:7, or SEQ ID NO:8.

A further embodiment of the invention includes a method for introducing a HCV replicon into a cell. The method produces a cell comprising a replicating chimeric HCV sub-genomic viral replicon. A preferred embodiment a cell of the invention comprising a HCV sub-genomic replicon further comprising all of the non-structural HCV genes and none of the structural HCV genes.

A further embodiment of the invention is a method of screening for compounds that modulate viral replication comprising the steps of administering a test compound to a cell comprising a replicating chimeric HCV sub-genomic viral replicon and determining whether said test compound modulates replication of said subgenomic replicon.

Another embodiment of the invention includes a method of screening for compounds that inhibit viral replication comprising the steps of administering a test compound to a cell comprising a replicating chimeric HCV sub-genoric viral replicon and determining whether said test compound inhibits replication of said chimeric sub-genoric viral replicon.

### BRIEF DESCRIPTION OF THE DRAWINGS

FIG. 1 shows a comparative sequence alignment of 3′UTR coding region from genotype 1a and 1b strains.

FIG. 2 schematically depicts the novel replicon constructs in comparison with the prototype pHCVrep1b(BB7). Replicon pHCVrep1b(BB7) contains noncoding sequences in thin lines and protein coding sequences in boxes (neo=G418 resistance gene; NS3 to NS5B=nonstructrual protein genes). The arrow on top of the figure shows the translation start codon AUG and the NS5A adaptive mutation Serine 1179 Isoleucine. The arrows below the Figure indicate the relevant restriction sites in the DNA plasmid used for sub-cloning. B, Bsr GI; RI, Eco RI; M, Mfe I. Stripped boxes indicate DNA sequences derived from pCVJ4, Shaded boxes indicate sequences derived from pCVH77. 5′ and 3′ NTR sequences are indicated while the nucleotide changes in 3′NTR are pointed out by the nucleotide under the 3′NTR. Replication capability was indicated by + and − signs at the left of the figure.

FIG. 3 depicts Huh-7 cell colony formation. Colonies formed after selection in G418 containing media for over four weeks were stained with 5% crystal violet in methanol and photographed.

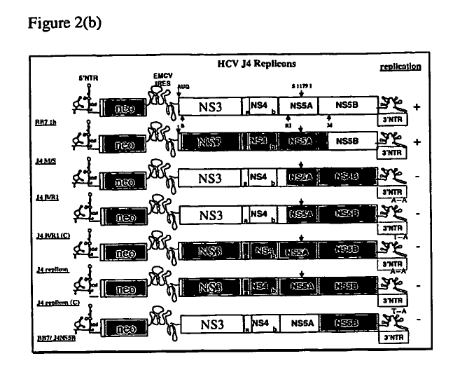

FIG. 4 depicts a Western blot of stable replicon cells expressing NS5A. Total replicon cell protein was separated on 8% SDS-PAGE, transferred to nitrocellulose paper and probed with anti-NS5A serum. Replicon cell A serves as positive control, while Huh-7 cell as negative control. The molecular weight is indicated.

FIG. 5 illustrates an analysis of replicon genomic DNA by PCR. Genomic DNA from each replicon cell line was purified and used as substrates in PCR reactions to amplify either GDAPH or Neo resistance gene. While in both H77 1A (F1) and J4 M/S cells, DNA for GAPDH was amplified, indicating that the good quality of genomic DNA, no Neo DNA was detected. For positive control of the reaction, amplification of neo DNA was from a neo plasmid DNA.

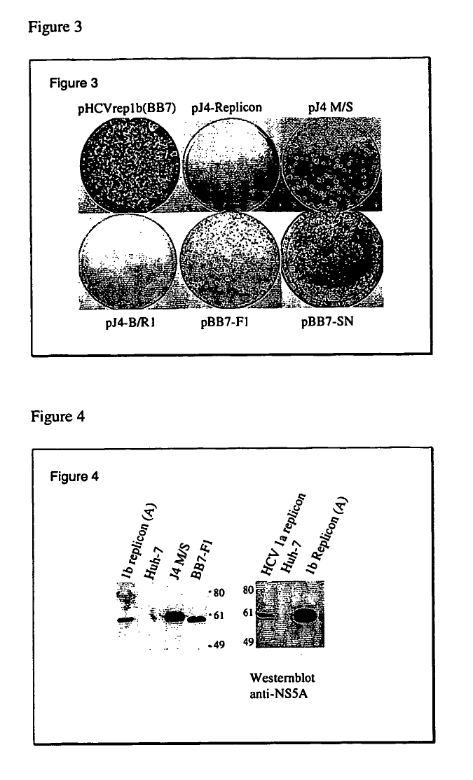

FIG. 6 illustrates a genome copy number determination by TaqMan analysis.

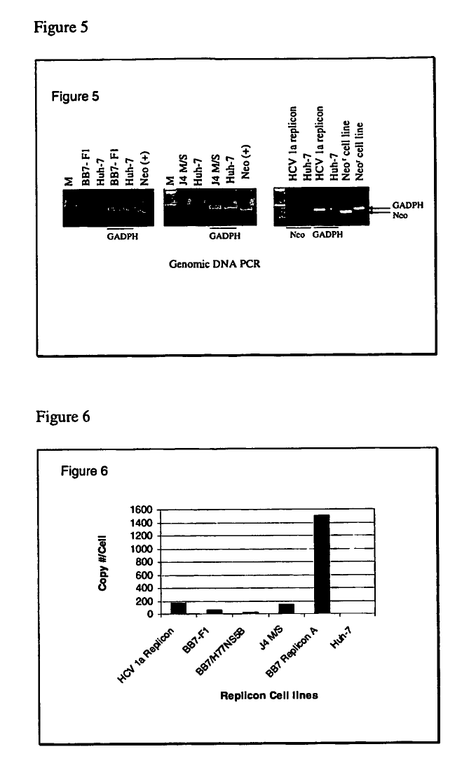

### DETAILED DESCRIPTION OF THE INVENTION

Through molecular cloning and tissue culture technologies, the present invention produced functionally stable cell lines expressing and replicating replicon RNA consisting of sequences from HCV genotype 1a, strain H77, within the prototype genotype 1b replicon, strain BB7, backbone. The type 1a replicon system is characterized in detail and discussed herein.

Specifically, expression of HCV viral proteins, NS3, NS5A, and NS5B, were detected by Western blot and in situ immunofluoresence analysis. Positive- and negative-strand replicon RNA was quantified by TaqMan and full length RNA was confirmed by northern blot analysis. Susceptibility to interferon and antiviral agents was shown to be similar when comparing with the type 1b replicon system. Lastly, cDNA was generated from replicon cell RNA by RT-PCR, and as many as 18 clones were sequenced to identify adaptive mutation(s) not present in the parental H77 sequence. The non-structural genes NS3, NS5A, and NS5B contained four mutations. While the two mutations in NS3 were found in all clones, the mutations in NS5A and NS5B were always present in a same subset of clones.

Huh-7 cell-based replicon systems for HCV were developed, in which the nonstructural proteins stably replicate sub-genomic viral RNA. (Lohmann et al., Science 285:110 (1999) and Blight et al., Science 290:1972 (2000)). Although two specific strains of genotype 1b, (HCV-N and HCV-BB7), have been confirmed to be functional in Huh-7 cells, it is not clear why other strains of genotype 1b are unable to replicate. The present invention is based, in part, on understanding the interplay of how certain adaptive mutations confer competence in Huh-7 cells for one viral strain, yet altogether different mutations are critical for a different viral strain would facilitate the creation of functional replicons for HCV genotypes 1 to 6.

These functional chimeric replicons are valuable tools that enable one skilled in the art to adapt known replicon systems to other HCV genotypes, such as HCV type 1a strain H77 (Yanagi, M. et. al. 1997) and HCV type 1b strain HC-J4 (Yanagi, M. et al. 1998), which have not been reported as successful. The reported cDNA for genotype 1b, strain J4, was constructed using the 5′ and 3′ untranslated regions of a genotype 1a strain (Yanagi, M. et al. 1998). The infectivity of three full-length cDNA clones was tested by direct injection of RNA transcripts into the liver of a chimpanzee and only one of the three clones was found to be infectious. The infectious clone coding region contained three amino acid changes from the parental J4 strain. Moreover, heterogeneity of the 3′UTR was examined and several changes compared to the parental genotype 1a 3′UTR were identified (nt 9407, 9399, poly U-UC region varied in length and complexity, and several point mutations in the conserved region of the 3′UTR. Although not mentioned by these authors, it is provided by this invention that these in vivo-selected changes indicate that the generation of functional HCV genomes requires a specific NS5B polymerase sequence (e.g., J4 strain polymerase sequence) to be linked in cis to its cognate 3′UTR sequences (e.g., J4 strain 3′UTR sequence). Replication of negative strand by NS5B initiates at the 3′UTR, and coordination of specific sequences in these two regions directs function. The present invention further provides generation of chimeric replicons containing genotype 1b sequence from the J4 strain that are nonfunctional when paired with 3′UTR sequences from genotype 1a strains. The invention thus explains the lack of reported J4 strain replicons to date, but in no way is the invention limited by this hypothesis.

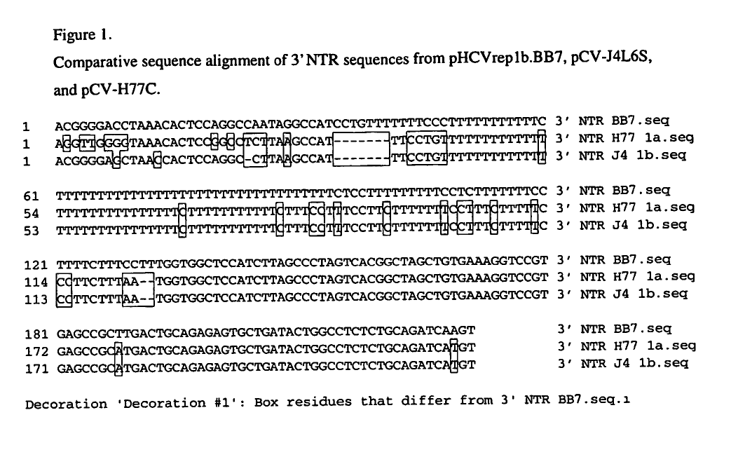

The present invention provides certain sub-genomic replicon constructs. A replicon comprising both the polyprotein coding sequences and 3′UTR sequences from a genotype 1a, strain H77, cDNA is functional, based on the in cis pairing of NS5B and 3′UTR from same strain. Moreover, functional replicons are provided wherein all polyprotein coding sequences except NS5B were replaced by sequences of genotype 1b, strain J4, while leaving NS5B and 3′UTR sequences intact from genotype 1b sequences reported in the BB7 1b functional replicon. Lastly, based on the 3′UTR sequences in FIG. 1, replicons containing solely J4 strain sequences function if the genotype 1a 3′UTR from the infectious cDNA is replaced with the 3′UTR from genotype 1b, strain J4. Data confirming this result is provided elsewhere herein.

In addition, the hybrid clones of the present invention retained a portion of the 5′ sequence of the non-structural gene NS3, genotype 1b, of the BB7 replicon. The first 225 nucleotides of genotype 1b replaced the corresponding 5′ end of the non-structural gene NS3 of either genotype 1a or genotype 1b, J4 strain.

The present invention discloses a panel of constructed BB7 strain chimeras, among others. These chimeras comprise sequences from the infectious strains J4, type 1b or H77, type 1a. The J4/BB7 chimeric replicons comprising coding sequence for NS3, NS4A, NS4B, NS5A, and 132 amino acids of NS5B from J4 and the remainder of the replicon from BB7 efficiently replicated in Huh-7 cells. However, replacement of BB7 NS5B and 3′NTR with J4 strain sequences failed to result in stable cell line generation, although transient replication was not ruled out. The H77/BB7 chimeric replicons containing NS3, NS4A, and NS4B sequences from H77 supported replication in Huh-7 cells, although replacement of BB7 NS5A and the N-terminal 132 amino acids of NS5B with H77 sequences rendered this replicon deficient. Lastly, replacing a portion of BB7 NS5B and 3′NTR elements with H77 sequences also did not confer stable replication in Huh-7 cells. Surprisingly, a chimeric replicon whereby the entire BB7 NS5B was replaced with H77 sequences resulted in efficient colony formation and stable replication in Huh-7 cells. Adaptive mutations were found in the H77 NS5B region of this replicon. Such replicons could be used for cell-based testing of antiviral agents, among other uses, against NS3 and NS5B from strains J4 and H77.

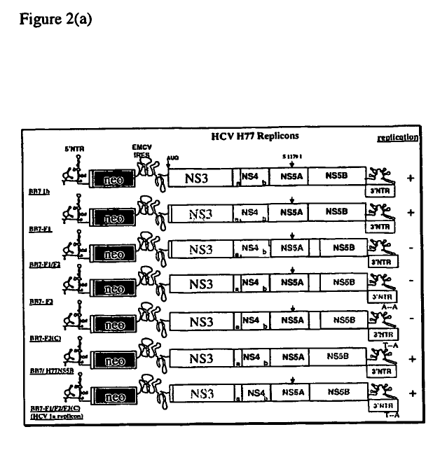

In the present invention, certain chimeric replicon constructs were generated from pHCVrep1b(BB7) with sequences substituted with either type 1aHCV H77 sequence or by HCV type 1b J4 strain sequence. Rather than trying to replace only the NS5B coding region, as other groups have attempted but failed to achieve, the present invention was based, in part, on the methodical replacement of sections of the pHCVrep1b(BB7) nonstructural genes with 1a or J4 1b sequences. Replicon RNA prepared in vitro for the majority of these chimeras (BB7-F1/F2, BB7-F3, BB7-F3(C), J4B/R1, J4 B/R1 (C), J4 replicon, J4 replicon (c), and BB7/J4NS5B, see FIG. 2) failed to yield stable cell lines after transfection into Huh-7 cells. However, surprisingly, constructs BB7-F1, BB7/H77NS5B, HCV1a replicon and J4 MIS were able to stably replicate and confer resistance to G418. The methods for constructing these replicons are described herein.

Evaluation of the functional replicon constructs demonstrated certain findings that are, in certain embodiments, important for chimeric replicon generation of multiple genotypes and subtypes: (1) A portion of NS5B, starting at residue 133, from strain BB7 genotype 1b can confer replication, albeit at low levels and (2) the 3′NTR sequences from BB7 can confer replication of the replicon, albeit also at low levels. Both the 3′NTR and a portion of residues from NS5B are, in certain embodiments, important components which together control replication of the sub-genomic RNA in Huh-7 cells. This finding indicates that inclusion of nonstructural gene sequences from other strains and genotypes results in a functional replicon if sequences for NS5B and 3′NTR region are from the same strain, for example BB7.

The invention further provides that other NS5B sequences can replace BB7 NS5B sequence, if paired with their cognate 3′NTR sequence elements. Moreover, certain sequence changes in the BB7 3′NTR, compared to changes in the J4 3′NTR indicate the basis of NS5B:3′NTR interaction, and thereby confer stable replication in Huh-7 cells. FIG. 1 shows an alignment of 3′NTR sequences from different strains.

Various definitions are used throughout this document. Most words have the meaning that would be attributed to those words by one skilled in the art. Words specifically defined either below or elsewhere in this document have the meaning provided in the context of the present invention as a whole and as typically understood by those skilled in the art.

As used herein, the term ♭replicon” refers to a viral nucleic acid that is capable of directing the generation of copies of itself. As used herein, the term “replicon” includes RNA as well as DNA, and hybrids thereof. For example, double-stranded DNA versions of HCV genomes can be used to generate a single-stranded RNA transcript that constitutes an HCV replicon. Generally, a viral replicon contains the complete genome of the virus. “Sub-genomic replicon,” as used herein, refers to a viral nucleic acid that contains something less than the full complement of genes and other features of the viral genome, yet is still capable of directing the generation of copies of itself. For example, the sub-genomic replicons of HCV described below contain most of the genes for the non-structural proteins of the virus, but are missing most of the genes coding for the structural proteins. Sub-genomic replicons are capable of directing the expression of all of the viral genes necessary for the replication of the viral sub-genome, replication of the sub-genomic replicon, without the production of viral particles.

An HCV sub-genomic replicon, may be derived from any of the various HCV strains and isolates, such as, but not limited to, any of the isolates from genotypes 1, 2, 3, 4, 5 or 6 of HCV. Moreover, the various genes included in the sub-genomic replicon can be derived from different strains. The complete genotypes of many of these strains are known. See, e.g., U.S. Pat. No. 6,150,087 and GenBank Accession Nos. AJ238800 and AJ238799, International Publication Nos. WO 89/04669; WO 90/11089; and WO 90/14436. Moreover, the genes included in the sub-genomic replicon may be full-length, fragments or variants of the native sequence, so long as the sub-genomic replicon remains capable of expressing the viral genes necessary for replication thereof, without producing viral particles. Thus, for example, the genes included in the sub-genomic replicon may be homologous to the native genes.

The phrase “stably replicating” as used herein in reference to the sub-genomic replicons means the steady, continuous generation of new sub-genomic replicons in the cells into which initial sub-genomic replicon transcripts are introduced, as well as their progeny cells. The transfected cells continue to proliferate, and the sub-genomic replicons continue to replicate.

The term “cell” as used herein refers to single cells as well as to the collection of cells in culture derived from a single progenitor cell, otherwise referred to as cell lines.

As used in this specification and the claims, the singular forms “a,” “an” and “the” include plural references unless the content clearly dictates otherwise. Thus, for example, reference to “a cell” includes a mixture of two or more cells.

Many different cell types can be used to practice the invention. In a preferred embodiment of the invention, the cell used is a human liver cell. More preferably, the cell is a hepatocellular carcinoma-derived cell. In a non-limiting example, the cell is Huh-7 (Nakabayashi et al., 1982, Cancer Res., 42:3858-3863; Seki et al., 1999, Hepatogastroenterology, 46:2812-2817). In another non-limiting example, the cell is HepG2 (U.S. Pat. No. 4,393,133). Other cell lines in which the invention may be practised include, but are not limited to, myc immortalized human liver cell lines, and primary cultures of fetal hepatocytes (Sanchez et al., 1995, J. Cell Physiol, 165:398-405).

Another aspect of the invention provides methods of screening for compounds that modulate replication of viral RNAs either directly or indirectly. Compounds can be screened for their effect on the replication of sub-genomic viral replicons in the cells of the invention by treating the cells with test compounds. Compounds that target the viral genes and/or proteins involved in the replication of sub-genomic viral replicons can be identified in screens of the invention where an HCV sub-genomic replicon can stably replicate.

The cells of the present invention can be used to identify compounds that inhibit viral RNA replication, and hence, viral replication, or to identify compounds that enhance viral RNA replication, and hence, viral replication. In particular, compounds identified as having inhibitory effects on the replication of HCV sub-genomic replicons will be candidates for use as drugs in the treatment of HCV infection and disease. Compounds exhibiting replication-enhancing activities will be candidates for use in the development of further cellular and animal model systems of HCV replication.

There are a variety of HCV targets for test compounds, including, but not limited to, HCV internal ribosomal entry sites, HCV NS3 serine proteinase, NS3 RNA helicase, NS5B RNA dependent RNA polymerase, and other HCV non-structural proteins. For example, compounds may interfere with the process of viral replicon replication by interfering with the viral proteins that are critical to RNA replication, all of which are translated off of the transcripts being generated in HCV sub-genomic replicon-containing cells of the present invention.

When a selectable drug resistance marker is included in the sub-genomic replicon, compounds can be assessed for their ability to sensitize cells to the selectable drug, i.e., to render the cells sensitive to the drug that was used to select them. Test cultures where cells die off are indicative of compounds that interfere with replicon replication, because loss of the drug-selectable replicon renders the cells sensitive to that particular drug. Where, for example, a neo resistance marker is used in conjunction with the viral sub-genomic replicon, loss of the neo-selectable, sub-genomic replicon will render the cells sensitive to G418.

As used herein, the term “compound” means any identifiable chemical or molecule, including, but not limited to small molecules, peptides, polypeptides, proteins, sugars, nucleotides, or nucleic acids. Such compounds can be natural or synthetic.

As used herein, the term “modulates” in reference to host replication activity means results in a change in the amount, quality, or effect of a particular response or activity. Both increases and decreases in the response or activity are included.

As used herein, the term “chimeric” means a molecule of RNA, DNA, or protein that has resulted from recombination, or has resulted from DNA from two sources fused or spliced together.

The invention is further illustrated by way of the following examples which are intended to elucidate the invention. These examples are not intended, nor are they to be construed, as limiting the scope of the invention. It will be clear that the invention may be practised otherwise that as particularly described herein. Numerous modifications and variations of the present invention are possible in view of the teachings herein and, therefore, are within the scope of the invention.

### EXAMPLES

### Example 1

### Construction of Replicons Comprising HCV Type 1a H77 Sequence

The present invention utilized PCR, in which pCV-H77C DNA was used as template to amplify a DNA fragment (F1) which contains NS3, starting at amino acid 76, NS4A, NS4B, and the N terminal of NS5A, ending at amino acid 148. The primers are designed according to the pCV-H77C DNA sequence with only one nucleotide change in each of the 5′ and 3′ primers. A single T to C change in the 5′ primer BG1000 incorporated a BsrGI restriction site while a single T to C change in the 3′ primer BG0002 created an Eco RI restriction site. Neither changes in the DNA sequence caused changes in the encoded amino acid sequences. The PCR product was digested with Eco RI and Bsr GI restriction enzymes and cloned into pHCVrep1b(BB7) vector that has been digested with the same enzymes. The resulting chimeric construct, pBB7-F1 was selected based on the lack of a Mlu I restriction site in the substituted H77 sequence and confirmed by DNA sequencing analysis.

PCR was utilized to amplify DNA fragment F2 by using H77 DNA as template and the primers BG1001 and BG1004. F2 extends from within NS5A (starting at amino acid 149) to NS5B (ending at amino acid 132), encompassing the S1179I adpative mutation. BG1001 is complementary to BG1002 and contained a single A to G nucletide change and an Eco RI restriction site. Similarly, a T to A change was built in BG1004 from the H77 sequence to create an Mfe I restriction site. To make the S1179I adaptive mutation, the 1.3 kb F2 was digested with Eco RI and MfeI and cloned into the corresponding sites in pLitmus 38 and subjected to oligonucleotide-directed mutagenesis with 1aS9484I and 1aS9484I R. The mutagenized fragment was subsequently cloned into pBB7-F1 that has been digested with Eco RI and Mfe I to produce pBB7-F1/F2.

An additional DNA fragment was amplified from H77 DNA by PCR to generate an F3(c) fragment containing H77 HCV 1a sequence from NS5B, at the end of F2, starting at amino acid 133, to the 3′ end of HCV genome. The 5′ primer BG1003 contains an MfeI site and the 3′ primer RB8000 contains a Spe I restriction site followed by a Sca I site. The ScaI site marks the end of the HCV genome in that after Sca I digestion and transcription the authentic RNA 3′ was produced. The RB8000 oligonucleotide has two changes from H77 in sequence, an A to T change at the −3 position and A to T change at the −44 position. The changes reflected the DNA sequence in pBB7 and preserved the base-pairing structure of the 3′ X tail (Yanagi, M et al. 1999). F3(c) fragment was digested with Mfe I and Spe I and purified from agose gels. It was ligated into pBB7-F1/F2 vector that was digested with Mfe I and Spe I to generate pBB7-F1I/F2/F3(c) or HCV 1A replicon. The F3 (C) fragment was also directly cloned into pHCVrep1b(BB7) to replace the corresponding DNA to make pBB7-F3(c). Similarly, Mfe I-Spe I fragment without the single nucleotide change F3 was cloned into pHCVrep1b(BB7) to generate pBB3-F3.

A replicon containing HCV type 1a NS5B, pBB7/H77NS5B, was constructed in multiple steps. First, a unique Sna BI site was introduced into pHCVrep1b(BB7) at the end of the NS5B gene. To accomplish this, two DNA fragments were amplified by using pHCVrep1b(BB7) as template and the primer pairs: rb6000 CGTCTGCTGCTCGATGTCCTAC and RB7801 3′ [SEQ ID NO:15] CTCCCCCAACCGATGAACGGGTACGTAAACACTCCAGGCCAATAG; BG1005 [SEQ ID NO: 16] GCACTAGTACTTGATCTGCAGAGAGGC and RB7801 5′ [SEQ ID NO: 17] CTATTGGCCTGGAGTGTTTACGTACCCGTTCATCGGTTGGGGGAG. [SEQ ID NO: 18] RB7801 5′ and RB7801 3′ are complementary primers derived from sequences at the end of NS5B. Two nucleotide sequences (underlined sequences) were changed from the BB7 HCV sequence so that a Sna BI site is created. The sequence changes do not affect the NS5B sequence since they are 3′ to the NS5B coding region. By using RB7801 3′ and rb6000, a 1.4 kb NS5B fragment was generated. By using RB7801 5′ and BG1005, a 220 bp 3′NTR sequence was generated. The two fragments were purified, annealed and the annealed product used as templates in PCR reactions to generate NS5B-3′NTR fragment. This fragment was digested with Bc1 I and Spe I and the resulting DNA ligated into pBB7 vector that were digested with the same restriction enzymes. The resulting plasmid, designated pBB7-SN, was confirmed to contain a Sna BI restriction site at the end of NS5B by restriction digestion and DNA sequencing. The NS5B gene fragment from pBB7-SN DNA was then replaced with other NS5B genes by cloning into the Bc1I and Sna BI restriction sites. Replicon RNA transcribed from pBB7-SN can replicate efficiently when electroporated into Huh-7 cells. To generate pBB7/H77NS5B, the HCV type 1a H77 NS5B gene was first amplified by using H77 DNA and primers RB7801 3′ and 1A 10501 CCTGGACAGGCGCACTGATCACC [SEQ ID NO: 19]. Two nucleotides were changed from the type 1a sequence to create a Bcl I site which caused a V to I substitution in H77 NS5B. After digestion with Bcl I and Sna BI, the H77 NS5B fragment was ligated to pBB7-SN vector prepared by digestion with the same restriction enzymes. Replacement of BB7 NS5B by H77 NS5B was confirmed by the introduction of an Eco RI restriction site. Similarly, pBB7/J4NS5B was constructed by cloning into pBB7-SN PCR fragments generated by using the primer pairs rb6000 and RB7801 3′ and pCVJ4L6S DNA.

For synthesis of all other hybrid J4 replicons, multiple cloning steps were used. First, pCVJ4L6S DNA was digested with BsrG I and Mfe I and the 4.3 kb DNA fragment encoding NS3 (from amino acid 75), NS4A, 4B, NS5A, and the N terminal 132 amino acids of NS5B was purified from agrose gels. The fragment was ligated with pHCVrep1b(BB7) vector digested with the same restriction enzymes to generate pBB7-J4. pBB7-J4 was distinguished from pHCVrep1b(BB7) by Hpa I digestion. pBB7-J4 has only one Hpal site while pHCVrep I b(BB7) has two. To put together the J4 replicon, primers J4-10861: GAGGACTTGCTGGAAGACACTG [SEQ ID NO:20] and BB7980: CAGGAGTACTTGATCTGCAGAGAGGC [SEQ ID NO:21] was used in PCR reactions to amplify the J4 DNA fragment 3′ to the Mfe I site. The DNA was digested with Mfe I and Sca I restriction enzymes and cloned into pBB7-J4 to create pJ4/BB7. To add the NS5A adaptation mutation S11791 in pHCVrep1b(BB7) to pJ4/BB7, the 1.3 kb Eco RI to Mfe I DNA fragment encoding part of NS5A surrounding the S11791 was subcloned into pLitmus 38. The resulting plasmid was subjected to oligonucleotide-directed mutagenesis to introduce the S11791 change by using the oligos J4-9841T and J49841TR. The mutagenized Eco RI-Mfe I fragment was used to replace the wildtype fragment in pJ4/BB7 to generate pJ4replicon. Regions containing the mutation in pJ4replicon were sequenced and both the J4 sequences and the S1179I mutation were confirmed. The pJ4replicon DNA also has the single nucleotide change (A to T at −40 position) in its 3′NTR X tail. This was fixed by first generating a PCR fragment by using the primer pairs BB8000 and J4-10861 and then replace the Mfe I-Spe I fragment in pJ4 replicon with the PCR fragment. The replicon with the corrected 3′ X tail is designated pJ4 replicon(c). The Mfe I-Spe I and BsrGI-Eco RI fragments from pHCVrep1b(BB7) were used to replace the corresponding sequence in pJ4 replicon(c) to make pJ4M/S and pJ4 B/R1 (c), respectively. The BsrGI-EcoRI fragment from pHCVrep1b(BB7) was also used on pJ4replicon to make pJ4 B/RI, which differs from pJ4B/RI(c) by a mutation in the 3′ X tail.

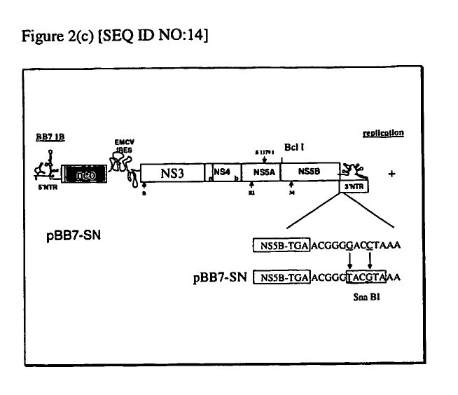

RNA transcribed from the above replicon DNA was electroporated into Huh-7 cells according to the procedures in the examples. G418 resistant cells were obtained by incubating the cells in G418-containing media. Once the cells were amplified, total cellular protein was separated on SDS-PAGE and probed by NS5A serum to assess whether HCV proteins were expressed. As predicted, all three replicon cell lines expressed NS5A as shown on figure. Genomic DNA and RNA were also isolated from each replicon cell lines. PCR reactions on genomic DNA were carried out to assess the possibility that the G418 resistance was due to expression of the phosphatransferase from integrated neo resistant gene into the Huh-7 cell genome. As shown in FIG. 4, while the PCR reaction amplified a fragment of neo DNA when using genomic DNA from a neo resistant cell line was used as substrate, no neo specific product was amplified from equivalent amount of genomic DNA from pJ4 M/S, pBB7-F1 and pHCVrep1b(BB7). However, when the same genomic DNA was used to amplify a GAPDH DNA fragment, the expected sized DNA was amplified readily, suggesting that the genomic DNA is of good quality. Therefore, the date indicate that it is unlikely that the resistance is due to integration of residue DNA in our RNA preparation into the genome of Huh-7 cells.

Next, replicon RNA was quantified by TaqMan. Real-time PCR was conducted with primers and probes in the neo gene on total RNA isolated from the replicon cells and on standard neo containing DNA. Copy numbers of the replicon were calculated based on standard curve generated from known amount of in vitro transcribed RNA. J4 M/S contained about 147 copies/cell, BB7-F1 contained 57 copies/cell, HCV 1a replicon has 165 copies/cell, BB7/H77NS5B has 29 copies/cell. For a positive control, HCV BB7 replicon that has 1500 copies/cell while Huh-7 cells served as negative controls.

### Example 2

### Cells, Media, DNA Cloning

Huh-7 cell lines were cultured in Dulbecco's Modified Eagle Media (DMEM) (Invitrogen #11965-084) containing, 10% fetal calf serum (FCS) (JRH Biosciences #12103-78P), 1% penicillin-streptomycin (P-S) (Invitrogen #15140-122), 1% non-essential amino acids (NEs) (Invitrogen #11140-050), and 1 mg/ml Geneticin (herein “G418”) (Invitrogen 11811-023).

### Example 3

### Construction of Hybrid H77 Replicons

PCR was carried out to amplify a DNA fragment (F1) which contains NS3 (from amino acid 76), NS4A, 4B and the N terminal of NS5A (148 amino acids) by using HCV-H type 1a DNA as substrate. The 100 ul reaction contains 1× pfu PCR buffer (Strategene), 0.25 uM BG1000, 0.25 uM BG1002 primers, 10 ng HCV type 1A DNA, 2 units turbo pfu DNA polymerase. The PCR conditions were set at 95 C for 30 seconds, 60 C for 30 seconds, 72C for 3 minutes for 25 cycles. The DNA product was purified from an agrose gel by Qiagen Quick gel kit, digested with BsrGI and EcoRI restriction enzymes. After purification, the DNA fragment was ligated into pHCVrep1b(BB7) vector DNA that was previously digested with BsrGI and EcoRI enzymes. The recombinant plamids were distinguished from background pHCVrep1b(BB7) plamids by DNA digestion with Mlu I. pHCVrep1b(BB7) has a Mlu I site in the substituted region, while recombinant pBB7 F1 lacks the Mlu I site. pH77 F1 DNA was prepared and sequence confirmed to contain type 1A sequences.

PCR reactions were carried out to amplify another DNA fragment (F2) by using H77 DNA as template and the primers BG1001 and BG1004. F2 extends from within NS5A to the 132 amino acid of NS5B, encompassing the S1179I adaptive mutation. BG1000 is complementary to BG 1002, thus containing a single nucleotide change, A to G, and an Eco RI restriction site. Similarly, a T to A change was built in BG1004 from the H77 sequence to create a Mfe I restriction site. The 1.3 kb PCR product was digested with Eco RI and MfeI and cloned into the corresponding sites in pLitmus38 and subjected to oligonucleotide-directed mutagenesis with the oligos J4-9841T and J49841TR. The mutagenized fragment was subsequently cloned into pBB7-F1 that has been digested with Eco RI and Mfe I to produce pBB7-F1/F2.

Another DNA fragment was amplified from H77 DNA by PCR to generate a F3(c) fragment which contains H77 HCV 1A sequence from the end of F2 to the 3′ end of HCV genome. The primers are BG1003, which was designed to contain an Eco RI site and RB8000, which contain a Spe I restriction site right next to a Sca I site. The ScaI site marks the end of the HCV genome in that after Sca I digestion and transcription the authentic RNA 3′ was produced. The oligonucleotide has two changes from H77 in sequence, an A to T change at the minus 3 position and T to A change at the minus 44 position. The changes reflected the DNA sequence in pBB7 and preserved the base-pairing structure of the 3′ X tail (Yanagi, M et al. 1999). F3(c) fragment was digested with Mfe I and Spe I and purified from agarose gel. Then ligated into pBB7-F1/F2 vector that was digested with Mfe I and Spe I to generate pBB7-F1/F2/F3(c) or HCV 1A replicon. The F3(c) fragment was also ligated into pBB7 vector to generate pBB7-F3(c). Another construct was generated by cloning an F3 fragment containing the mismatch in the 98 nt x-tail. The −44 position was left to have the T, but the −3 position was changed to T to create the Sca I site. This construct is designated pBB7-F3.

TABLE 1
The restriction sites and there positions as
used in the construction of the replicons.

| | |
|---|---|
| BsrGI | nt 2024 (NS3 aa#75) |
| Eco RI | nt 5083 (NS5A aa#148) |
| Mfe I | nt 6383 (NS5B aa#132) |
| Sna BI | nt 7764 (3′NTR) |

TABLE 2
Summary of nucleotide and amino acid changes
in HCV 1a replicon and BB7/H77NS5.
Changes in HCV 1a replicon:

| | | |
|---|---|---|
| Position | Nucleotide Change | Amino acid change |

I. BB7 adaptive mutation:

| | | |
|---|---|---|
| 5336 | G to T | NS5A S232I |

II. HCV 1a adaptive muation:

| | | |
|---|---|---|
| 2797 | T to C | NS3 S332P |
| 3550 | A to G | NS3 K583E |
| 4682 | A to T | NS5A E14V |
| 6520 | G to A | NS5B V179I |
| 6936 | C to A | none |
| 7356 | G to A | none |

III. changes made during cloning:

| | | |
|---|---|---|
| 2028 | T to C (create BsrGI site) | none |
| 5088 | T to C (create Eco RI site) | none |
| 6387 | A to T (create Mfe I site) | none |

Mutations in BB7/H77NS5B:

| | | |
|---|---|---|
| 6012 | C to G | NS5B V11I |
| 6013 | G to A | NS5B V11I |
| 7024 | G to T | NS5B A348S |

### Example 4

### Construction of Hybrid J4 Replicons

pHCV J4 DNA was digested with BsrG I and Mfe I and the 4.3 kb DNA fragment containing type 1B NS3 (from amino acid 75), NS4a, 4B, NS5A, and the N terminal 132 amino acids of NS5B was purified from agrose gels. The fragment was cloned into pHCVrep1b(BB7) vector DNA that was digested previously with the same restriction enzyme to generate pJ4M/S*. To add the NS5A adaptation mutation S11791 in pHCVrep1b(BB7) to pJ4 M/S*, a 1.3 kb Eco RI to Mfe I DNA fragment from pHCVrep1b(BB7) was subcloned into pLitmus 38 and subjected to oligonucleotide-directed mutagenesis. The oligonucleotides J4-9841T: CTTTAGCCAGCTCATCAGCTATCCAGTTGTCTGCGCCTTC [SEQ ID NO:22] and J49841TR:GAAGGCGCAGACAACTGGATAGCTGATGAGCTGGCTAAAC [SEQ ID NO:23] were used in PCR reactions according to the manufacturer's directions. The mutagenized Eco RI-MfeI fragment was then cloned into pJ4M/S* to generate pJ4M/S. DNA of pJ4M/S was sequenced to confirm both the J4 sequences and the S1179I mutation.

### Example 5

### In Vitro Transcription and DNase Digest of Replicon RNA

Replicon plasmid DNA was linearized with Sca I (New England Biolabs) using 1 U/1□g DNA for 1 hour at 37° C. The linearization of the plasmid generates the authentic 3′ end of the HCV 3′NTR and provides the template to generate the appropriate RNA transcript from the upstream T7 promoter. The DNA was subsequently purified in two phenol:chloroform:IAA extractions, followed by an EtOH precipitation. The linearized DNA pellet was dried and resuspended in nuclease-free water for use in an in vitro transcription reaction. The in vitro transcription reaction was performed using the Megascript T7 Kit (Ambion). A total of 1 ug of linearized DNA was aliquoted into a solution containing a final concentration of 1× Reaction Buffer, 1× Enzyme Mix and 7.5 mM of ATP, UTP, CTP and GTP. The reaction was incubated at 37° C. for 4 hours in an air incubator. Following the incubation, 1 U/1□g DNA of RQ1 DNase enzyme (Promega) was added to the reaction and incubated at 37° C. for 2 hours. The newly synthesized RNA was purified using the RNeasy Kit (Qiagen). Following the manufacturer's protocol, the RNA was bound to the RNeasy column and an additional DNase digest was performed using 1 U of RNase-free DNase (Qiagen) for 1 hour at 25° C. The RNA was eluted in nuclease free water and stored at −80° C.

### Example 6

### Electroporation of RNA into Huh-7 Cells

Five □g of replicon RNA was electroporated into 5×10⁶ cells of the hepatoma cell line, Huh-7. Briefly, Huh-7 cells were passed the day before the electroporation and grown to 50% confluency. Huh-7 cells were pelleted, washed, counted and resuspended to a final concentration of 1×10⁷ cells/ml in Opti-MEM media (Invitrogen). A total of 5 □g of RNA was aliquoted into a pre-chilled cuvette on ice. A 0.2 ml aliquot of the cell suspension was then added to the cuvette containing the RNA. The RNA was immediately electroporated into the Huh-7 cells using the Gene Pulser (Bio-Rad), adjusted to deliver one pulse of 0.2 kV, 100 ohms and 960 □FD. The cuvettes were chilled on ice for 10 minutes to allow the cells to recover. The cells were plated on a 100 mm tissue culture plate in complete media. Selection media containing either 0.5 to 1.0 mg/ml of G418 (Invitrogen) was added to the cells 24-48 hours post-electroporation.

### Example 7

### Analysis of Replicons by Western Blot Analysis

Total cell lysates were harvested from replicon cells in 1.1× LDS Buffer (Invitrogen). The lysates were heated for 10 minutes in the presence of a reducing agent. The lysates were run on a 10% Bis-Tris NuPage polyachrylamide gel (Invitrogen) in 1× MOPS buffer as recommended by the manufacturer. Protein transfer to PVDF (Invitrogen) membrane was performed using the Semi-Dry Trans Blot System (Bio-Rad). Following transfer, the blot was rinsed once with PBS containing 0.5% Tween-20 (PBS-Tween). The blot was blocked in PBS-Tween containing 5% non-fat milk for 1 hour at room temperature with gently agitation. The blot was rinsed quickly with two washes of PBS-Tween, followed by two ten minute PBS-Tween washes with gentle agitation. The primary antibody incubation to detect HCV NS5a protein utilized an □-NS5a rabbit polyclonal antibody dilued 1:2500 in PBS-Tween. The incubation was conducted for 1 hour at room temperature with gentle agitation. The □-NS5a antibody was removed and the blot was rinsed quickly with two washes of PBS-Tween, followed by two 10 minute PBS-Tween washes with gentle agitation. A secondary antibody incubation was performed using a HRP conjugated □-rabbit IgG antibody (Amersham) diluted in 1:5000 in PBS-Tween. Again, the incubation was done at room temperature for 1 hour with gentle agitation. Following the secondary antibody incubation, the blot was rinsed quickly with two washes of PBS-Tween, followed by two ten minute PBS-Tween washes with gentle agitation. The blot was incubated in Super Signal Chemiluminescent Reagent (Pierce) following the manufacturer's protocol and exposed to film (Amersham).

### Example 8

### Isolation of Genomic DNA

Genomic DNA was isolated using DNeasy Tissue Kit (Qiagen) according to the manufacturer's protocol. The experiments to determine possible genomic incorporation of the neomycin resistance gene was performed using PCR. Primers Ralf Neo 5′TCA AGA CCG ACC TGT CCG GTG CCC [SEQ ID NO:24] and Ralf Neo 3′CTT GAG CCT GGC GAA CAG TTC GGC [SEQ ID NO:25] to amplify a 380 bp product form 100 ng of genomic DNA. All PCR reactions utilized 2.5 U of PFU Turbo polymerase (Stratagene) per 100 ul reaction. The PCR reactions contained a final primer and dNTP concentration of 250 nM and 100 □M, respectively. In addition, the final buffer concentrations comprised 20 mM Tris-HCl (pH 8.8), 2 mM MgSO₄, 10 mM KCl, 10 mM (NH4)₂SO₄, 0.1% Triton X-100, and 0.1 mg/ml nuclease free BSA. Control primers GAPDH for ACC ACA GTC CAT GCC ATC AC [SEQ ID NO:26] and GAPDH rev TCC ACC ACC CTG TTG CTG TA [SEQ ID NO:27] were used to demonstrate the presence of intact genomic DNA in each PCR reaction.

### Example 9

### Analysis of Replicon Copy Number by TaqMan

Cells were trypinized and counted to determine cell number. The total cellular RNA was isolated using RNeasy Mini Kit (Qiagen) following the manufacturer's protocol. The RNA was stored at −80° C. or assayed by TaqMan quantitative RT-PCR. For analysis by TaqMan PCR, a master mix was prepared using TaqMan EZ RT-PCR Kit (Applied Biosystems #403028), TaqMan PDAR Control Reagent Human Cyclophilin (Applied Biosystems #4310883E), custom primer Neo^{R} fwd CCG GCT ACC TGC CCA TTC [SEQ ID NO:28] (Invitrogen), custom primer Neo^{R} rev CCA GAT CAT CCG ATC GAC AAG [SEQ ID NO:29] (Invitrogen), and custom probe Neo^{R} probe 5′FAM-ACA TCG CAT CGA GCG AGC ACG TAC-TAMRA 3′ [SEQ ID NO:30] (Biosource International). Master mix concentrations were as follows: 1× TaqMan EZ Buffer, 3 mM Mn(Oac)₂, 0.3 mM dATP, 0.3 mM dCTP, 0.3 mM dGTP, 0.6 mM dUTP, 0.2 mM Neo^{R} fwd, 0.2 mM Neo^{R} Rev, 0.1 mM Neo^{R} probe, 1× Cyclophilin Mix, 0.1 Unit/□l rTth DNA Polymerase, 0.01 Unit/□l AmpErase UNG, and H₂O to 40 □l. A total of 40 □l of master mix was added to wells of a 96-tube optical plate (Applied Biosystems #N801-0560), followed by 10 □l of RNA sample at a concentration of 5 ng/□l. The plate was covered with an optical adhesive cover (Applied Biosystems #4311971) and mixed by repeated inversion several times, followed by a brief centrifuge spin. The plate was placed in an AB17700 (Applied Biosystems) and the entire plate was set to FAM dye layer for unknown, and to the VIC dye layer for control. The cycling parameters were set to 50° C, 2 min.; 60° C., 30 min.; 95° C., 5 min.; (94° C., 20 sec.; 55° C., 1 min.) 40 cycles, with an exposure time of 10 milliseconds and spectral compensation. Data was analyzed by leaving the baseline to default level on both layers, and set a threshold to an equal value for both dye layers. The data was exported to Microsoft Excel for further analysis. To determine replicon copy number per cell, a linear regression curve for the known copy number Neo^{R} standards was established by plotting the Cycle Threshold values (Ct) versus the log of copy number for each Neo^{R} standard. The copy number for each replicon sample was calculated by taking the sample's Ct value, minus the line intercept, divided by the slope of the line.

TABLE 3
Polynucleotide Sequences

(A) Sequences from HCV H77 Replicons
BB7-F1 [SEQ ID NO: 2]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTCTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCGTGAAT
GAACTGCAGGACGAGGCAGCGCGGCTATCGTGGCTGGCCACGACGGGCGTTCC
TTGCGCAGCTGTGCTCGACGTTGTCAC
TGAAGCGGGAAGGGACTGGCTGCTATTGGGCGAAGTGCCGGGGCAGGATCTCC
TGTCATGTCACGTTGCTCCTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCCT
GCTTGCCGAATATCATGGTGGAAAATGGCCGCTTTTCTGGATTCATCGACTGTG
GCCGGCTGGGTGTGGCGGACCGCTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGCTGCATCATCAGTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTAcACCAATGTGGACCAAGACCTTGTGGG
CTGGCCCGCTCCTCAAGGTTCCCGCT
CATTGACACCCTGTACCTGCGGCTCCTCGGACCTTTACCTGGTCACGAGGCACG
CCGATGTCATTCCCGTGCGCCGGCGA
GGTGATAGCAGGGGTAGCCTGCTTTCGCCCCGGCCCATTTCCTACTTGAAAGGC
TCCTCGGGGGGTCCGCTGTTGTGCCC
CGCGGGACACGCCGTGGGCCTATTCAGGGCCGCGGTGTGCACCCGTGGAGTGG
CTAAAGCGGTGGACTTTATCCCTGTGG
AGAACCTAGGGACAACCATGAGATCCCCGGTGTTCACGGACAACTCCTCTCCAC
CAGCAGTGCCCCAGAGCTTCCAGGTG
GCCCACCTGCATGCTCCCACCGGCAGCGGTAAGAGCACCAAGGTCCCGGCTGC
GTACGCAGCCCAGGGCTACAAGGTGTT
GGTGCTCAACCCCTCTGTTGCTGCAACGCTGGGCTTTGGTGCTTACATGTCCAAG
GCCCATGGGGTTGATCCTAATATCA
GGACCGGGGTGAGAACAATTACCACTGGCAGCCCCATCACGTACTCCACCTACG
GCAAGTTCCTTGCCGACGGCGGGTGC
TCAGGAGGTGCTTATGACATAATAATTTGTGACGAGTGCCACTCCACGGATGCC
ACATCCATCTTGGGCATCGGCACTGT
CCTTGACCAAGCAGAGACTGCGGGGGCGAGACTGGTTGTGCTCGCCACTGCTAC
CCCTCCGGGCTCCGTCAGTGTGTCCC
ATCCTAACATCGAGGAGGTTGCTCTGTCCACCACCGGAGAGATCCCCTTTTACG
GCAAGGCTATCCCCCTCGAGGTGATC
AAGGGGGGAAGACATCTCATCTTCTGCCACTCAAAGAAGAAGTGCGACGAGCT
CGCCGCGAAGCTGGTCGCATTGGGCAT
CAATGCCGTGGCCTACTACCGCGGTCTTGACGTGTGTGTCATCCCGACCAGCGG
CGATGTTGTCGTCGTGTCGACCGATG
CTCTCATGACTGGCTTACCGGCGACTTCGACTCTGTGATAGACTGCAACACGT
GTGTCACTCAGACAGTCGATTTCAGC
CTTGACCCTACCTTTACCATTGAGACAACCACGCTCCCCCAGGATGCTGTCTCCA
GGACTCAACGCCGGGGCAGGACTGG
CAGGGGGAAGCCAGGCATCTATAGATTTGTGGCACCGGGGGAGCGCCCCTCCG
GCATGTTCGACTCGTCCGTCCTCTGTG
AGTGCTATGACGCGGGCTGTGCTTGGTATGAGCTCACGCCCGCCGAGACTACAG
TTAGGCTACGAGCGTACATGAACACC
CCGGGGCTTCCCGTGTGCCAGGACCATCTTGAATTTTGGGAGGGCGTCTTTACG
GGCCTCACTCATATAGATGCCCACTT
TTTATCCCAGACAAAGCAGAGTGGGGAGAACTTTCCTTACCTGGTAGCGTACCA
AGCCACCGTGTGCGCTAGGGCTCAAG
CCCCTCCCCCATCGTGGGACCAGATGTGGAAGTGTTTGATCCGCCTTAAACCCA
CCCTCCATGGGCCAACACCCCTGCTA
TACAGACTGGGCGCTGTTCAGAATGAAGTCACCCTGACGCACCCAATCACCAAA
TACATCATGACATGCATGTCGGCCGA
CCTGGAGGTCGTCACGAGCACCTGGGTGCTCGTTGGCGGCGTCCTGGCTGCTCT
GGCCGCGTATTGCCTGTCAACAGGCT
GCGTGGTCATAGTGGGCAGGATCGTCTTGTCCGGGAAGCCGGCAATTATACCTG
ACAGGGAGGTTCTCTACCAGGAGTTC
GATGAGATGGAAGAGTGCTCTCAGCACTTACCGTACATCGAGCAAGGGATGAT
GCTCGCTGAGCAGTTCAAGCAGAAGGC
CCTCGGCCTCGTGCAGACCGCGTCCCGCCATGCAGAGGTTATCACCCCTGCTGT
CCAGACCAACTGGCAGAAACTCGAGG
TCTTTTGGGCGAAGCACATGTGGAATTTCATCAGTGGGATACAATACTTGGCGG
GCCTGTCAACGCTGCCTGGTAACCCC
GCCATTGCTTCATTGATGGCTTTTACAGCTGCCGTCACCAGCCCACTAACCACTG
GCCAAACCCTCCTCTTCAACATATT
GGGGGGGTGGGTGGCTGCCCAGCTCGCCGCCCCCGGTGCCGCTACTGCCTTTGT
GGGTGCTGGCCTAGCTGGCGCCGCCA
TCGGCAGCGTTGGACTGGGGAAGGTCCTCGTGGACATTCTTGCAGGGTATGGCG
CGGGCGTGGCGGGAGCTCTTGTAGCA
TTCAAGATCATGAGCGGTGAGGTCCCCTCCACGGAGGACCTGGTCAATCTGCTG
CCCGCCATCCTCTCGCCTGGAGCCCT
TGTAGTCGGTGTGGTCTGCGCAGCAATACTGCGCCGGCACGTTGGCCCGGGCGA
GGGGGCAGTGCAATGGATGAACCGGC
TAATAGCCTTCGCCTCCCGGGGGAACCATGTTTCCCCCACGCACTACGTGCCGG
AGAGCGATGCAGCCGCCCGCGTCACT
GCCATACTCAGCAGCCTCACTGTAACCCAGCTCCTGAGGCGACTGCATCAGTGG
ATAAGCTCGGAGTGTACCACTCCATG
CTCCGGTTCCTGGCTAAGGGACATCTGGGACTGGATATGCGAGGTGCTGAGCGA
CTTTAAGACCTGGGTGAAAGCCAAGC
TCATGCCACAACTGCCTGGGATTCCCTTTGTGTCCTGCCAGCGCGGGTATAGGG
GGGTCTGGCGAGGAGACGGCATTATG
CACACTCGCTGCCACTGTGGAGCTGAGATCACTGGACATGTCAAAAACGGGAC
GATGAGGATCGTCGGTCCTAGGACCTG
CAGGAACATGTGGAGTGGGACGTTCCCCATTAACGCCTACACCACGGGCCCTG
TACTCCCCTTCCTGCGCCGAACTATA
AGTTCGCGCTGTGGAGGGTGTCTGCAGAGGAATACGTGGAGATAAGGCGGGTG
GGGGACTTCCACTACGTATCGGGTATG
ACTACTGACAATCTTAAATGCCCGTGCCAGATCCCATCGCCCGAATTCTTCACA
GAAGTGGATGGGGTGCGGTTGCACAG
GTACGCTCCAGCGTGCAAACCCCTCCTACGGGAGGAGGTCACATTCCTGGTCGG
GCTCAATCAATACCTGGTTGGGTCAC
AGCTCCCATGCGAGCCCGAACCGGACGTAGCAGTGCTCACTTCCATGCTCACCG
ACCCCTCCCACATTACGGCGGAGACG
GCTAAGCGTAGGCTGGCCAGGGGATCTCCCCCCTCCTTGGCCAGCTCATCAGCT
ATCCAGCTGTCTGCGCCTTCCTTGAA
GGCAACATGCACTACCCGTCATGACTCCCCGGACGCTGACCTCATCGAGGCCAA
CCTCCTGTGGCGGCAGGAGATGGGCG
GGAACATCACCCGCGTGGAGTCAGAAAATAAGGTAGTAATTTTGGACTCTTTCG
AGCCGCTCCAAGCGGAGGAGGATGAG
AGGGAAGTATCCGTTCCGGCGGAGATCCTGCGGAGGTCCAGGAAATTCCCTCGA
GCGATGCCCATATGGGCACGCCCGGA
TTACAACCCTCCACTGTTAGAGTCCTGGAAGGACCCGGACTACGTCCCTCCAGT
GGTACACGGGTGTCCATTGCCGCCTG
CCAAGGCCCCTCCGATACCACCTCCACGGAGGAAGAGGACGGTTGTCCTGTCAG
AATCTACCGTGTCTTCTGCCTTGGCG
GAGCTCGCCACAAAGACCTTCGGCAGGTCCGAATCGTCGGCCGTCGACAGCGG
CACGGCAACGGCCTCTCCTGACCAGCC
CTCCGACGACGGCGACGCGGGATCCGACGTTGAGTCGTACTCCTCCATGCCCCC
CCTTGAGGGGGAGCCGGGGGATCCCG
ATCTCAGCGACGGGTCTTGGTCTACCGTAAGCGAGGAGGCTAGTGAGGACGTCG
TCTGCTGCTCGATGTCCTACACATGG
ACAGGCGCCCTGATCACGCCATGCGCTGCGGAGGAAACCAAGCTGCCCATCAA
TGCACTGAGCAACTCTTTGCTCCGTCA
CCACAACTTGGTCTATGCTACAACATCTCGCAGCGCAAGCCTGCGGCAGAAGAA
GGTCACCTTTGACAGACTGCAGGTCC
TGGACGACCACTACCGGGACGTGCTCAAGGAGATGAAGGCGAAGGCGTCCACA
GTTAAGGCTAAACTTCTATCCGTGGAG
GAAGCCTGTAAGCTGACGCCCCCACATTCGGCCAGATCTAAATTTGGCTATGGG
GCAAAGGACGTCCGGAACCTATCCAG
CAAGGCCGTTAACCACATCCGCTCCGTGTGGAAGGACTTGCTGGAAGACACTGA
GACACCAATTGACACCACCATCATGG
CAAAAAATGAGGTTTTCTGCGTCCAACCAGAGAAGGGGGGCCGCAAGCCAGCT
CGCCTTATCGTATTCCCAGATTTGGGG
GTTCGTGTGTGCGAGAAAATGGCCCTTTACGATGTGGTCTCCACCCTCCCTCAG
GCCGTGATGGGCTCTTCATACGGATT
CCAATACTCTCCTGGACAGCGGGTCGAGTTCCTGGTGAATGCCTGGAAAGCGAA
GAAATGCCCTATGGGCTTCGCATATG
ACACCCGCTGTTTTGACTCAACGGTCACTGAGAATGACATCCGTGTTGAGGAGT
CAATCTACCAATGTTGTGACTTGGCC
CCCGAAGCCAGACAGGCCATAAGGTCGCTCACAGAGCGGCTTTACATCGGGGG
CCCCCTGACTAATTCTAAAGGGCAGAA
CTGCGGCTATCGCCGGTGCCGCGCGAGCGGTGTACTGACGACCAGCTGCGGTAA
TACCCTCACATGTTACTTGAAGGCCG
CTGCGGCCTGTCGAGCTGCGAAGCTCCAGGACTGCACGATGCTCGTATGCGGAG
ACGACCTTGTCGTTATGTGTGAAAGC
GCGGGGACCCAAGAGGACGAGGCGAGCCTACGGGCCTTCACGGAGGCTATGAC
TAGATAGTCTGCCCCCCCTGGGGACCC
GCCAAACCAGAATACGACTTGGAGTTGATAACATCATGCTCCTCCAATGTGTC
AGTCGCGCACGATGCATCTGGCAAAA
GGGTGTACTATCTCACCCGTGACCCCACCACCCCCCTTGCGCGGGCTGCGTGGG
AGACAGCTAGACACACTCCAGTCAAT
TCCTGGCTAGGCAACATCATCATGTATGCGCCCACCTTGTGGGCAAGGATGATC
CTGATGACTCATTTCTTCTCCATCCT
TCTAGCTCAGGAACAACTTGAAAAAGCCCTAGATTGTCAGATCTACGGGGCCTG
TTACTCCATTGAGCCACTTGACCTAC
CTCAGATCATTCAACGACTCCATGGCCTTAGCGCATTTTCACTCCATAGTTACTC
TCCAGGTGAGATCAATAGGGTGGCT
TCATGCCTCAGGAAACTTGGGGTACCGCCCTTGCGAGTCTGGAGACATCGGGCC
AGAAGTGTCCGCGCTAGGCTACTGTC
CCAGGGGGGGAGGGCTGCCACTTGTGGCAAGTACCTCTTCAACTGGGCAGTAA
GGACCAAGCTCAAACTCACTCCAATCC
CGGCTGCGTCCCAGTTGGATTATCCAGCTGGTTCGTTGCTGGTTACAGCGGGG
GAGACATATATCACAGCCTGTCTCGT
GCCCGACCCCGCTGGTTCATGTGGTGCCTACTCCTACTTTCTGTAGGGGTAGGCA
TCTATCTACTCCCCAACCGATGAAC
GGGGACCTAAACACTCCAGGCCAATAGGCCATCCTGTTTTTTTCCCTTTTTTTTT
TTCTTTTTTTTTTTTTTTTTTTTTT
TTTTTTTTTTTTCTCCTTTTTTTTTCCTCTTTTTTTCCTTTTCTTTCCTTTGGTGGCT
CCATCTTAGCCCTAGTCACGGC
TAGCTGTGAAAGGTCCGTGAGCCGCTTGACTGCAGAGAGTGCTGATACTGGCCT
CTCTGCAGATCAAGT
BB7-F1/F2: [SEQ ID NO: 3]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTGTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGCTATCGTGGCTGGCCACGACGGGCGTTCC
TTGCGCAGCTGTGCTCGACGTTGTCAC
TGAAGCGGGAAGGGACTGGCTGCTATTGGGCGAAGTGCCGGGGCAGGATGTCC
TGTCATCTCACGTTGCTCCTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCCT
GCTTGCCGAATATCATGGTGGAAAATGGCCGCTTTTCTGGATTCATCGACTGTG
GCCGGCTGGGTGTGGCGGACCGCTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTGTGGAAAGAGTCAAATGGCTGTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGCTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTAcACCAATGTGGACCAAGACCTTGTGGG
CTGGCCCGCTCCTCAAGGTTCCCGCT
CATTGACACCCTGTACCTGCGGCTCCTCGGACCTTTACCTGGTCACGAGGCACG
CCGATGTCATTCCCGTGCGCCGGCGA
GGTGATAGCAGGGGTAGCCTGCTTTCGCCCCGGCCCATTTCCTACTTGAAAGGC
TCGTCGGGGGGTCCGCTGTTGTGCCC
CGCGGGACACGCCGTGGGCCTATTCAGGGCCGCGGTGTGCACCCGTGGAGTGG
CTAAAGCGGTGGACTTTATCCCTGTGG
AGAACCTAGGGACAACCATGAGATCCCCGGTGTCACGGACAACTCCTCTCCAC
CAGCAGTGCCCCAGAGCTTCCAGGTG
GCCCACCTGCATGCTCCCACCGGCAGCGGTAAGAGCACCAAGGTCCCGGCTGC
GTACGCAGCCCAGGGCTACAAGGTGTT
GGTGCTCAACCCCTCTGTTGCTGCAACGCTGGGCTTTGGTGCTTACATGTCCAAG
GCCCATGGGGTTGATCCTAATATCA
GGACCGGGGTGAGAACAATTACCACTGGCAGCCCCATCACGTACTCCACCTACG
GCAAGTTCCTTGCCGACGGCGGGTGC
TCAGGAGGTGCTTATGACATAATAATTTGTGACGAGTGCCACTCCACGGATGCC
ACATCCATCTTGGGCATCGGCACTGT
CCTTGACCAAGCAGAGACTGCGGGGGCGAGACTGGTTGTGCTCGCCACTGCTAC
CCCTCCGGGCTCCGTCACTGTGTCCC
ATCCTAACATCGAGGAGGTTGCTCTGTCCACCACCGGAGAGATCCCCTTTTACG
GCAAGGCTATCCCCCTCGAGGTGATC
AAGGGGGGAAGACATCTCATCTTCTGCCACTCAAAGAAGAAGTGCGACGAGCT
CGCCGCGAAGCTGGTCGCATTGGGCAT
CAATGCCGTGGCCTACTACCGCGGTCTTGACGTGTCTGTCATCCCGACCAGCGG
CGATGTTGTCGTCGTGTCGACCGATG
CTCTCATGACTGGCTTTACCGGCGACTTCGACTCTGTGATAGACTGCAACACGT
GTGTCACTCAGACAGTCGATTTCAGC
CTTGACCCTACCTTTACCATTGAGACAACCACGCTCCCCCAGGATGCTGTCTCCA
GGACTCAACGCCGGGGCAGGACTGG
CAGGGGGAAGCCAGGCATCTATAGATTTGTGGCACCGGGGGAGCGCCCCTCCG
GCATGTTCGACTCGTCCGTCCTCTGTG
AGTGCTATGACGCGGGCTGTGCTTGGTATGAGCTCACGCCCGCCGAGACTACAG
TTAGGCTACGAGCGTACATGAACACC
CCGGGGCTTCCCGTGTGCCAGGACCATCTTGAATTTTGGGAGGGCGTCTTTACG
GGCCTCACTCATATAGATGCCCACTT
TTTATCCCAGACAAAGCAGAGTGGGGAGAACTTTCCTTACCTGGTAGCGTACCA
AGCCACCGTGTGCGCTAGGGCTCAAG
CCCGTCCCCCATCGTGGGACCAGATGTGGAAGTGTTTGATCCGCCTTAAACCCA
CCCTCCATGGGCCAACACCCCTGCTA
TACAGACTGGGCGCTGTTCAGAATGAAGTCACCCTGACGCACCCAATCACCAAA
TACATCATGACATGCATGTCGGCCGA
CCTGGAGGTCGTCACGAGCACCTGGGTGCTCGTTGGCGGCGTCCTGGCTGCTCT
GGCCGCGTATTGCCTGTCAACAGGCT
GCGTGGTCATAGTGGGCAGGATCGTCTTGTCCGGGAAGCCGGCAATTATACCTG
ACAGGGAGGTTCTCTACCAGGAGTTC
GATGAGATGGAAGAGTGCTCTCAGCACTTACCGTACATCGAGCAAGGGATGAT
GCTCGCTGAGCAGTTCAAGCAGAAGGC
CCTCGGCCTCCTGCAGACCGCGTCCCGCCATGCAGAGGTTATCACCCCTGCTGT
CCAGACCAACTGGCAGAAACTCGAGG
TCTTTTGGGCGAAGCACATGTGGAATTTCATCAGTGGGATACAATACTTGGCGG
GCCTGTCAACGCTGCCTGGTAACCCC
GCCATTGCTTCATTGATGGCTTTTACAGCTGCCGTCACCAGCCCACTAACCACTG
GCCAAACCCTCCTCTTCAACATATT
GGGGGGGTGGGTGGCTGCCCAGGTCGCCGCCCCCGGTGCCGCTACTGCCTTTGT
GGGTGCTGGCCTAGCTGGCGCCGCCA
TCGGCAGCGTTGGACTGGGGAAGGTCCTCGTGGACATTCTTGCAGGGTATGGCG
CGGGCGTGGCGGGAGCTCTTGTAGCA
TTCAAGATCATGAGCGGTGAGGTCCCCTCCACGGAGGACCTGGTCAATCTGCTG
CCCGCCATCCTCTCGCCTGGAGCCCT
TGTAGTCGGTGTGGTCTGCGCAGCAATACTGCGCCGGCACGTTGGCCCGGGCGA
GGGGGCAGTGCAATGGATGAACCGGC
TAATAGCCTTCGCCTCCCGGGGGAACCATGTTTCCCCCACGCACTACGTGCCGG
AGAGCGATGCAGCCGCCCGCGTCACT
GCCATACTCAGCAGCCTCACTGTAACCCAGCTCCTGAGGCGACTGCATCAGTGG
ATAAGCTCGGAGTGTACCACTCCATG
CTCCGGTTCCTGGCTAAGGGACATCTGGGACTGGATATGCGAGGTGCTGAGCGA
CTTTAAGACCTGGCTGAAAGCCAAGC
TCATGCCACAACTGCCTGGGATTCCCTTTGTGTCCTGCCAGCGCGGGTATAGGG
GGGTCTGGCGAGGAGACGGCATTATG
CACACTCGCTGCCACTGTGGAGCTGAGATCACTGGACATGTCAAAAAACGGGAC
GATGAGGATCGTCGGTCCTAGGACCTG
CAGGAACATGTGGAGTGGGACGTTCCCCATTAACGCCTACACCACGGGCCCCTG
TACTCCCCTTCCTGCGCCGAACTATA
AGTTCGCGCTGTGGAGGGTGTCTGCAGAGGAATACGTGGAGATAAGGCGGGTG
GGGGACTTCCACTACGTATCGGGTATG
ACTACTGACAATCTTAAATGCCCGTGCCAGATCCCATCGCCCGAATTcTTCACAG
AATTGGACGGGGTGCGCCTACACAG
GTTTGCGCCCCCTTGCAAGCCCTTGCTGCGGGAGGAGGTATCATTCAGAGTAGG
ACTCCACGAGTACCCGGTGGGGTCGC
AATTACCTTGCGAGCCCGAACCGGACGTAGCCGTGTTGACGTCCATGCTCACTG
ATCCCTCCCATATAACAGCAGAGGCG
GCCGGGAGAAGGTTGGCGAGAGGGTCACCCCCTTCTATGGCCAGCTCCTCGGCT
AtCCAGCTGTCCGCTCCATCTCTCAA
GGCAACTTGCACCGCCAACCATGACTCCCCTGACGCCGAGCTCATAGAGGCTAA
CCTCCTGTGGAGGCAGGAGATGGGCG
GCAACATCACCAGGGTTGAGTCAGAGAACAAAGTGGTGATTCTGGACTCCTTCG
ATCCGCTTGTGGCAGAGGAGGATGAG
CGGGAGGTCTCCGTACCTGCAGAAATTCTGCGGAAGTCTCGGAGATTCGCCCGG
GCCCTGCCCGTCTGGGCGCGGCCGGA
CTACAACCCCCCGCTAGTAGAGACGTGGAAAAAGCCTGACTACGAACCACCTGT
GGTCCATGGCTGCCCGCTACCACCTC
CACGGTCCCCTCCTGTGCCTCCGCCTCGGAAAAAGCGTACGGTGGTCCTCACCG
AATCAACCCTATCTACTGCCTTGGCC
GAGCTTGCCACCAAAAGTTTTGGCAGCTCCTCAACTTCCGGCATTACGGGCGAC
AATACGACAACATCCTCTGAGCCCGC
CCCTTCTGGCTGCCCCCCCGACTCCGACGTTGAGTCCTATTCTTCCATGCCCCCC
CTGGAGGGGGAGCCTGGGGATCCGG
ATCTCAGCGACGGGTCATGGTCGACGGTCAGTAGTGGGGCCGACACGGAAGAT
GTCGTGTGCTGCTCAATGTCTTATTCC
TGGACAGGCGCACTCGTCACCCCGTGCGCTGCGGAAGAACAAAAACTGCCCAT
CAACGCACTGAGCAACTCGTTGCTACG
CCATCACAATCTGGTGTATTCCACCACTTCACGCAGTGCTTGCCAAAGGCAGAA
GAAAGTCACATTTGACAGACTGCAAG
TTCTGGACAGCCATTACCAGGACGTGCTCAAGGAGGTCAAAGCAGCGGCGTCA
AAAGTGAAGGCTAACTTGCTATCCGTA
GAGGAAGCTTGCAGCCTGACGCCCCCACATTCAGCCAAATCCAAGTTTGGCTAT
GGGGCAAAAGACGTCCGTTGCCATGC
CAGAAAGGCCGTAGCCCACATCAACTCCGTGTGGAAAGACCTTCTGGAAGACA
GTGTAACACCAATTGACACCACCATCA
TGGCAAAAAATGAGGTTTTCTGCGTCCAACCAGAGAAGGGGGGCCGCAAGCCA
GCTCGCCTTATCGTATTCCCAGATTTG
GGGGTTCGTGTGTGCGAGAAAATGGCCCTTTACGATGTGGTCTCCACCCTCCCT
CAGGCCGTGATGGGCTCTTCATACGG
ATTCCAATACTCTCCTGGACAGCGGGTCGAGTTCCTGGTGAATGCCTGGAAAGC
GAAGAAATGCCCTATGGGCTTCGCAT
ATGACACCCGCTGTTTTGACTCAACGGTCACTGAGAATGACATCCGTGTTGAGG
AGTCAATCTACCAATGTTGTGACTTG
GCCCCCGAAGCCAGACAGGCCATAAGGTCGCTCACAGAGCGGCTTTACATCGG
GGGCCCCCTGACTAATTCTAAAGGGCA
GAACTGCGGCTATCGCCGGTGCCGCGCGAGCGGTGTACTGACGACCAGCTGCG
GTAATACCCTCACATGTTACTTGAAGG
CCGCTGCGGCGTGTCGAGCTGCGAAGCTCCAGGAGTGCACGATGCTCGTATGCG
GAGACGACCTTGTCGTTATCTGTGAA
AGCGCGGGGACCCAAGAGGACGAGGCGAGCCTACGGGCCTTCACGGAGGCTAT
GAGTAGATACTCTGCCCCCCCTGGGGA
CCCGCCCAAACCAGAATACGACTTGGAGTTGATAACATCATGCTCCTCCAATGT
GTCAGTCGCGCACGATGCATCTGGCA
AAAGGGTGTACTATCTCACCCGTGACCCCACCACCCCCCTTGCGCGGGCTGCGT
GGGAGACAGCTAGACACACTCCAGTC
AATTCCTGGCTAGGCAACATCATCATGTATGCGCCCACCTTGTGGGCAAGGATG
ATCCTGATGACTCATTTCTTCTCCAT
CCTTCTAGCTCAGGAACAACTTGAAAAAGCCCTAGATTGTCAGATCTACGGGGC
CTGTTACTCCATTGAGCCACTTGACC
TACCTCAGATCATTCAACGACTCCATGGCCTTAGCGCATTTTCACTCCATAGTTA
CTCTCCAGGTGAGATCAATAGGGTG
GCTTCATGCCTCAGGAAACTTGGGGTACCGCCCTTGCGAGTCTGGAGACATCGG
GCCAGAAGTGTCCGCGCTAGGCTACT
GTCCCAGGGGGGGAGGGCTGCCACTTGTGGCAAGTACCTCTTCAACTGGGCAGT
AAGGACCAAGCTCAAACTCACTCCAA
TCCCGGCTGCGTCCCAGTTGGATTTATCCAGCTGGTTCGTTGCTGGTTACAGCGG
GGGAGACATATATCACAGCCTGTCT
CGTGCCCGACCCCGCTGGTTCATGTGGTGCCTACTCCTACTTTCTGTAGGGGTAG
GCATCTATCTACTCCCCAACCGATG
AACGGGGACCTAAACACTCCAGGCCAATAGGCCATCCTGTTTTTTTCCCTTTTTT
TTTTTCTTTTTTTTTTTTTTTTTTT
TTTTTTTTTTTTTTTCTCCTTTTTTTTTCCTCTTTTTTTCCTTTTCTTTCCTTTGGTGG
CTCCATCTTAGCCCTAGTCAC
GGGTAGCTGTGAAAGGTCCGTGAGCCGCTTGACTGCAGAGAGTGCTGATACTGG
CCTCTCTGCAGATCAAGT
BB7-F3: [SEQ ID NO: 4]
GCCAGCCCCCGATTGGGGGCGACACTCCACCATAGATCACTCCCGTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTCTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGCTATCGTGGCTGGCCACGACGGGCGTTCC
TTGCGCAGCTGTGCTCGACGTTGTCAC
TGAAGCGGGAAGGGACTGGCTGCTATTGGGCGAAGTGCCGGGGCAGGATCTCC
TGTCATGTCACCTTGCTCCTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCCT
GCTTGCCGAATATCATGGTGGAAAATGGCCGCTTTTCTGGATTCATCGACTGTG
GCCGGCTGGGTGTGGCGGACCGGTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGCTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTGGACCAGGACCTCGTCG
GCTGGCAAGCGCCCCCCGGGGCGCGTT
CCTTGACACCATGCACCTGCGGCAGCTCGGACCTTTACTTGGTCACGAGGCATG
CCGATGTCATTCCGGTGCGCCGGCGG
GGCGACAGCAGGGGGAGCGTAGTGTCCCCCAGGCCCGTCTCCTACTTGAAGGGC
TCTTCGGGCGGTCCACTGCTCTGCCC
CTCGGGGCACGCTGTGGGCATCTTTCGGGCTGCCGTGTGCACCCGAGGGGTTGC
GAAGGCGGTGGACTTTGTACCCGTCG
AGTCTATGGAAACCACTATGCGGTCCCCGGTCTTCACGGACAACTCGTCCCCTC
CGGCCGTACCGCAGACATTCCAGGTG
GCCCATCTACACGCCCCTACTGGTAGCGGCAAGAGCACTAAGGTGCCGGCTGCG
TATGCAGCCCAAGGGTATAAGGTGCT
TGTCCTGAACCCGTCCGTCGCCGCCACCCTAGGTTTCGGGGCGTATATGTCTAA
GGCACATGGTATCGACCCTAACATCA
GAACCGGGGTAAGGACCATCACCACGGGTGCCCCCATCACGTACTCCACCTATG
GCAAGTTTCTTGCCGACGGTGGTTGC
TCTGGGGGCGCCTATGACATCATAATATGTGATGAGTGCCACTCAACTGACTCG
ACCACTATCCTGGGCATCGGCACAGT
CCTGGACCAAGCGGAGACGGCTGGAGCGCGACTCGTCGTGCTCGCCACCGCTA
CGCCTCCGGGATCGGTCACCGTGCCAC
ATCCAAACATCGAGGAGGTGGCTCTGTCCAGCACTGGAGAAATCCCCTTTTATG
GCAAAGCCATCCCCATCGAGACCATC
AAGGGGGGGAGGCACCTCATTTTCTGCCATTCCAAGAAGAAATGTGATGAGCTC
GCCGCGAAGCTGTCCGGCCTCGGACT
CAATGCTGTAGCATATTACCGGGGCCTTGATGTATCCGTCATACCAACTAGCGG
AGACGTCATTGTCGTAGCAACGGACG
CTCTAATGACGGGCTTTACCGGCGATTTCGACTCAGTGATCGACTGCAATACAT
GTGTCACCCAGACAGTCGACTTCAGC
CTGGACCCGACCTTCACCATTGAGACGACGACCGTGCCACAAGACGCGGTGTCA
CGCTCGCAGCGGCGAGGCAGGACTGG
TAGGGGCAGGATGGGCATTTACAGGTTTGTGACTCCAGGAGAACGGCCCTCGG
GCATGTTCGATTCCTCGGTTCTGTGCG
AGTGCTATGACGCGGGCTGTGCTTGGTACGAGCTCACGCCCGCCGAGACCTCAG
TTAGGTTGCGGGCTTACCTAAACACA
CCAGGGTTGCCCGTCTGCCAGGACCATCTGGAGTTCTGGGAGAGCGTCTTTACA
GGCCTCACCCACATAGACGCCCATTT
CTTGTCCCAGACTAAGCAGGCAGGAGACAACTTCCCCTACCTGGTAGCATACCA
GGCTACGGTGTGCGCCAGGGCTCAGG
CTCCACCTCCATCGTGGGACCAAATGTGGAAGTGTCTCATACGGCTAAAGCCTA
CGCTGCACGGGCCAACGCCCCTGCTG
TATAGGCTGGGAGCCGTTCAAAACGAGGTTACTACCACACACCCCATAACCAAA
TACATCATGGCATGCATGTCGGCTGA
CCTGGAGGTCGTCACGAGCACCTGGGTGCTGGTAGGCGGAGTCCTAGCAGCTCT
GGCCGCGTATTGCCTGACAACAGGCA
GCGTGGTCATTGTGGGCAGGATCATCTTGTCCGGAAAGCCGGCCATCATTCCCG
ACAGGGAAGTCCTTTACCGGGAGTTC
GATGAGATGGAAGAGTGCGCCTCACACCTCCCTTACATCGAACAGGGAATGCA
GCTCGCCGAACAATTCAAACAGAAGGC
AATCGGGTTGCTGCAAACAGCCACCAAGCAAGCGGAGGCTGCTGCTCCCGTGGT
GGAATCCAAGTGGCGGACCCTCGAAG
CCTTCTGGGCGAAGCATATGTGGAATTTCATCAGCGGGATACAATATTTAGCAG
GCTTGTCCACTCTGCCTGGCAACCCC
GCGATAGCATCACTGATGGCATTCACAGCCTCTATCACCAGCCCGCTCACCACC
CAACATACCCTCCTGTTTAACATCCT
GGGGGGATGGGTGGCCGCCCAACTrGCTCCTCCCAGCGCTGCTTCTGCTTTCGT
AGGCGCCGGCATCGCTGGAGCGGCTG
TTGGCAGCATAGGCCTTGGGAAGGTGCTTGTGGATATTTTGGCAGGTTATGGAG
CAGGGGTGGCAGGCGCGCTCGTGGCC
TTTAAGGTCATGAGCGGCGAGATGCCCTCCACCGAGGACCTGGTTAACCTACTC
CCTGCTATCCTCTCCCCTGGCGCCCT
AGTCGTCGGGGTCGTGTGCGCAGCGATACTGCGTCGGCACGTGGGCCCAGGGG
AGGGGGCTGTGCAGTGGATGAACCGGC
TGATAGCGTTCGCTTCGCGGGGTAACCACGTCTCCCCCACGCACTATGTGCCTG
AGAGCGACGCTGCAGCACGTGTCACT
CAGATCCTCTCTAGTCTTACCATCACTCAGCTGCTGAAGAGGCTTCACCAGTGG
ATCAACGAGGACTGCTCCACGCCATG
CTCCGGCTCGTGGCTAAGAGATGTTTGGGATTGGATATGCACGGTGTTGACTGA
TTTCAAGACCTGGCTCCAGTCCAAGC
TCCTGCCGCGATTGCCGGGAGTCCCCTTCTTCTCATGTCAACGTGGGTACAAGG
GAGTCTGGCGGGGCGACGGCATCATG
CAAACCACCTGCCCATGTGGAGCACAGATCACCGGACATGTGAAAAACGGTTC
CATGAGGATCGTGGGGCCTAGGACCTG
TAGTAACACGTGGCATGGAACATTCCCCATTAACGCGTACACCACGGGCCCCTG
CACGCCCTCCCCGGCGCCAAATTATT
CTAGGGCGCTGTGGCGGGTGGCTGCTGAGGAGTACGTGGAGGTTACGCGGGTG
GGGGATTTCCACTACGTGACGGGCATG
ACCACTGACAACGTAAAGTGCCCGTGTCAGGTTCCGGCCCCCGAATTCTTCACA
GAAGTGGATGGGGTGCGGTTGCACAG
GTACGCTCCAGCGTGCAAACCCCTCCTACGGGAGGAGGTCACATTCCTGGTCGG
GCTCAATCAATACCTGGTTGGGTCAC
AGCTCCCATGCGAGCCCGAACCGGACGTAGCAGTGCTCACTTCCATGCTCACCG
ACCCCTCCCACATTACGGCGGAGACG
GCTAAGCGTAGGCTGGCCAGGGGATCTCCCCCCTCCTTGGCCAGCTCATCAGCT
ATCCAGCTGTCTGCGCCTTCCTTGAA
GGCAACATGCACTACCCGTCATGACTCCCCGGACGCTGACCTCATCGAGGCCAA
CCTCCTGTGGCGGCAGGAGATGGGCG
GGAACATCACCCGCGTGGAGTCAGAAAATAAGGTAGTAATTTTGGACTCTTTCG
AGCCGCTCCAAGCGGAGGAGGATGAG
AGGGAAGTATCCGTTCCGGCGGAGATCCTGCGGAGGTCCAGGAAATTCCCTCGA
GCGATGCCCATATGGGCACGCCCGGA
TTACAACCCTCCACTGTTAGAGTCCTGGAAGGACCCGGACTACGTCCCTCCAGT
GGTACACGGGTGTCCATTGCCGCCTG
CCAAGGCCCCTCCGATACCACCTCCACGGAGGAAGAGGACGGTTGTCCTGTCAG
AATCTACCGTGTCTTCTGCCTTGGCG
GAGCTCGCCACAAAGACCTTCGGCAGCTCCGAATCGTCGGCCGTCGACAGCGG
CACGGCAACGGCCTCTCCTGACCAGCC
CTCCGACGACGGCGACGCGGGATCCGACGTTGAGTCGTACTCCTCCATGCCCCC
CCTTGAGGGGGAGCCGGGGGATCCCG
ATCTCAGCGACGGGTCTTGGTGTACCGTAAGCGAGGAGGCTAGTGAGGACGTCG
TCTGCTGCTCGATGTCCTACACATGG
ACAGGCGCCCTGATCACGCCATGCGCTGCGGAGGAAACCAAGCTGCCCATCAA
TGCACTGAGCAACTCTTTGCTCCGTCA
CCACAACTTGGTCTATGCTACAACATCTCGCAGCGCAAGCCTGCGGCAGAAGAA
GGTCACCTTTGACAGACTGCAGGTCC
TGGACGACCACTACCGGGACGTGCTCAAGGAGATGAAGGCGAAGGCGTCCACA
GTTAAGGCTAAACTTCTATCCGTGGAG
GAAGCCTGTAAGCTGACGCCCCCACATTCGGCCAGATCTAAATTTGGCTATGGG
GCAAAGGACGTCCGGAACCTATCCAG
CAAGGCCGTTAACCACATCCGCTCCGTGTGGAAGGACTTGCTGGAAGACACTGA
GACACCAATtGACACTACCATCATGG
CCAAGAACGAGGTTTTCTGCGTTCAGCCTGAGAAGGGGGGTCGTAAGCCAGCTC
GTCTCATCGTGTTCCCCGACCTGGGC
GTGCGCGTGTGCGAGAAGATGGCCCTGTACGACGTGGTTAGCAAGCTCCCCCTG
GCCGTGATGGGAAGCTCCTACGGATT
CCAATACTCACCAGGACAGCGGGTTGAATTCCTCGTGCAAGCGTGGAAGTCCAA
GAAGACCCCGATGGGGTTCTCGTATG
ATACCCGCTGTTTTGACTCCACAGTCACTGAGAGCGACATCCGTACGGAGGAGG
CAATTTACCAATGTTGTGACCTGGAC
CCCCAAGCCCGCGTGGCCATCAAGTCCCTCACTGAGAGGCTTTATGTTGGGGGC
CCTCTTACCAATTCAAGGGGGGAAAA
CTGCGGCTACCGCAGGTGCCGCGCGAGCGGCGTACTGACAACTAGCTGTGGTAA
CACCCTCACTTGCTACATCAAGGCCC
GGGCAGCCTGTCGAGCCGCAGGGCTCCAGGACTGCACCATGCTCGTGTGTGGCG
ACGACTTAGTCGTTATCTGTGAAAGT
GCGGGGGTCCAGGAGGACGCGGCGAGCCTGAGAGCCTTCACGGAGGCTATGAC
CAGGTACTCCGCCCCCCCCGGGGACCC
CCCACAACCAGAATACGACTTGGAGCTTATAACATCATGCTCCTCCAACGTGTC
AGTCGCCCACGACGGCGCTGGAAAGA
GGGTCTACTACCTTACCCGTGACCCTACAACCCCCCTCGCGAGAGCCGCGTGGG
AGACAGCAAGACACACTCCAGTCAAT
TCCTGGCTAGGCAACATAATCATGTTTGCCCCCACACTGTGGGCGAGGATGATA
CTGATGACCCATTTCTTTAGCGTCCT
CATAGCCAGGGATCAGCTTGAACAGGCTCTTAACTGTGAGATCTACGGAGCCTG
CTACTCCATAGAACCACTGGATCTAC
CTCCAATCATTCAAAGACTCCATGGCCTCAGCGCATTTTCACTCCACAGTTACTC
TCCAGGTGAAATCAATAGGGTGGCC
GCATGCCTCAGAAAACTTGGGGTCCCGCCCTTGCGAGCTTGGAGACACCGGGCC
CGGAGCGTCCGCGCTAGGCTTCTGTC
CAGAGGAGGCAGGGCTGCCATATGTGGCAAGTACCTCTTCAACTGGGCAGTAA
GAACAAAGCTCAAACTCACTCCAATAG
CGGCCGCTGGCCGGCTGGACTTGTCCGGTTGGTTCACGGCTGGGTACAGCGGGG
GAGACATTTATCACAGCGTGTCTCAT
GCCCGGCCCCGCTGGTTCTGGTTTTGCCTACTCCTGCTCGCTGCAGGGGTAGGCA
TCTACCTCCTCCCCAACCGATGAAG
GTTGGGGTAAACACTCCGGCCTCTTAAGCCATTTCCTGTTTTTTTTTTTTTTTTTT
TTTTTTTTTTCTTTTTTTTTTTCT
TTCCTTTCCTTCTTTTTTTCCTTTCTTTTTCCCTTCTTTAATGGTGGCTCCATCTTA
GCCCTAGTCACGGCTAGCTGTGA
AAGGTCCGTGAGCCGCATGACTGCAGAGAGTGCTGATACTGGCCTCTGTGCAGA
TCAaGT
BB7-F3(C): [SEQ ID NO: 5]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTGTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGCTATCGTGGCTGGCCACGACGGGCGTTCC
TTGCGCAGCTGTGCTCGACGTTGTCAC
TGAAGCGGGAAGGGACTGGCTGCTATTGGGCGAAGTGCCGGGGCAGGATCTCC
TGTCATCTCACCTTGCTCCTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCCT
GCTTGCCGAATATCATGGTGGAAAATGGCCGCTTTTCTGGATTCATCGACTGTG
GCCGGCTGGGTGTGGCGGACCGCTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGCTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTGGACCAGGACCTCGTCG
GCTGGCAAGCGCCCCCCGGGGCGCGTT
CCTTGACACCATGCACCTGCGGCAGCTCGGACCTTTACTTGGTCACGAGGCATG
CCGATGTCATTCCGGTGCGCCGGCGG
GGCGACAGCAGGGGGAGCCTACTCTCCCCCAGGCCCGTCTCCTACTTGAAGGGC
TCTTCGGGCGGTCCACTGCTCTGCCC
CTCGGGGCACGCTGTGGGCATCTTTCGGGCTGCCGTGTGCACCCGAGGGGTTGC
GAAGGCGGTGGACTTTGTACCCGTCG
AGTCTATGGAAACCACTATGCGGTCCCCGGTCTTCACGGACAACTCGTCCCCTC
CGGCCGTACCGCAGACATTCCAGGTG
GCCCATCTACACGCCCCTACTGGTAGCGGCAAGAGCACTAAGGTGCCGGCTGCG
TATGCAGCCCAAGGGTATAAGGTGCT
TGTCCTGAACCCGTCCGTCGCCGCCACCCTAGGTTTCGGGGCGTATATGTCTAA
GGCACATGGTATCGACCCTAACATCA
GAACCGGGGTAAGGACCATCACCACGGGTGCCCCCATCACGTACTCCACCTATG
GCAAGTTTCTTGCCGACGGTGGTTGC
TCTGGGGGCGCCTATGACATCATAATATGTGATGAGTGCCACTCAAGTGACTCG
ACCACTATCCTGGGCATCGGCACAGT
CCTGGACCAAGCGGAGACGGCTGGAGCGCGACTCGTCGTGCTCGCCACCGCTA
CGCCTCCGGGATCGGTCACCGTGCCAC
ATCCAAACATCGAGGAGGTGGCTCTGTCCAGCACTGGAGAAATCCCCTTTTATG
GCAAAGCCATCCCCATCGAGACCATC
AAGGGGGGGAGGCACCTCATTTTCTGCCATTCCAAGAAGAAATGTGATGAGCTC
GCCGCGAAGCTGTCCGGCCTCGGACT
CAATGCTGTAGCATATTACCGGGGCCTTGATGTATCCGTCATACCAACTAGCGG
AGACGTCATTGTCGTAGCAACGGACG
CTCTAATGACGGGCTTTACCGGCGATTTCGACTCAGTGATCGACTGCAATACAT
GTGTCACCCAGACAGTCGACTTCAGC
CTGGACCCGACCTTCACCATTGAGACGACGACCGTGCCACAAGACGCGGTGTCA
CGCTCGCAGCGGCGAGGCAGGACTGG
TAGGGGCAGGATGGGCATTTACAGGTTTGTGACTCCAGGAGAACGGCCCTCGG
GCATGTTCGATTCCTCGGTTCTGTGCG
AGTGCTATGACGCGGGCTGTGCTTGGTACGAGCTCACGCCCGCCGAGACCTCAG
TTAGGTTGCGGGCTTACGTAAACACA
CCAGGGTTGCCCGTCTGCCAGGACCATCTGGAGTTCTGGGAGAGCGTCTTTACA
GGCCTCACCCACATAGACGCCCATTT
CTTGTCCCAGACTAAGCAGGCAGGAGACAACTTCCCCTACCTGGTAGCATACCA
GGCTACGGTGTGCGCCAGGGCTCAGG
CTCCACCTCCATCGTGGGACCAAATGTGGAAGTGTCTCATACGGCTAAAGCCTA
CGCTGCACGGGCCAACGCCCCTGCTG
TATAGGCTGGGAGCCGTTCAAAACGAGGTTACTACCACACACCCCATAACCAAA
TACATCATGGCATGCATGTCGGCTGA
CCTGGAGGTCGTCACGAGCACCTGGGTGCTGGTAGGCGGAGTCCTAGCAGCTCT
GGCCGCGTATTGCCTGACAACAGGCA
GCGTGGTCATTGTGGGCAGGATCATCTTGTCCGGAAAGCCGGCCATCATTCCCG
ACAGGGAAGTCCTTTACCGGGAGTTC
GATGAGATGGAAGAGTGCGCCTCACACCTCCCTTACATCGAACAGGGAATGCA
GCTCGCCGAACAATTCAAACAGAAGGC
AATCGGGTTGCTGCAAACAGCCACCAAGCAAGCGGAGGCTGCTGCTCCCGTGGT
GGAATCCAAGTGGCGGACCCTCGAAG
CCTTCTGGGCGAAGCATATGTGGAATTTCATCAGCGGGATACAATATTTAGCAG
GCTTGTCCACTCTGCCTGGCAACCCC
GCGATAGCATCACTGATGGCATTCACAGCCTCTATCACCAGCCCGCTCACCACC
CAACATACCCTCCTGTTTAACATCCT
GGGGGGATGGGTGGCCGCCCAACTTGCTCCTCCCAGCGCTGCTTCTGCTTTCGT
AGGCGCCGGCATCGCTGGAGCGGCTG
TTGGCAGCATAGGCCTTGGGAAGGTGCTTGTGGATATTTTGGCAGGTTATGGAG
CAGGGGTGGCAGGCGCGCTCGTGGCC
TTTAAGGTCATGAGCGGCGAGATGCCCTCCACCGAGGACCTGGTTAACCTACTC
CCTGCTATCCTCTCCCCTGGCGCCCT
AGTCGTCGGGGTCGTGTGCGCAGCGATACTGCGTCGGCACGTGGGCCCAGGGG
AGGGGGCTGTGCAGTGGATGAACCGGC
TGATAGCGTTCGCTTCGCGGGGTAACCACGTCTCCCCCACGCACTATGTGCCTG
AGAGCGACGCTGCAGCACGTGTCACT
CAGATCCTCTCTAGTCTTACCATCACTCAGCTGCTGAAGAGGCTTCACCAGTGG
ATCAACGAGGACTGCTCCACGCCATG
CTCCGGCTCGTGGCTAAGAGATGTTTGGGATTGGATATGCACGGTGTTGACTGA
TTTCAAGACCTGGCTCCAGTCCAAGC
TCCTGCCGCGATTGCCGGGAGTCCCCTTCTTCTCATGTCAACGTGGGTACAAGG
GAGTCTGGCGGGGCGACGGCATCATG
CAAACCACCTGCCCATGTGGAGCACAGATCACCGGACATGTGAAAAACGGTTC
CATGAGGATCGTGGGGCCTAGGACCTG
TAGTAACACGTGGCATGGAACATTCCCCATTAACGCGTACACCACGGGCCCCTG
CACGCCCTCCCCGGCGCCAAATTATT
CTAGGGCGCTGTGGCGGGTGGCTGCTGAGGAGTACGTGGAGGTTACGCGGGTG
GGGGATTTCCACTACGTGACGGGCATG
ACCACTGACAACGTAAAGTGCCCGTGTCAGGTTCCGGCCCCCGAATTCTTCACA
GAAGTGGATGGGGTGCGGTTGCACAG
GTACGCTCCAGCGTGCAAACCCCTCCTACGGGAGGAGGTCACATTCCTGGTCGG
GCTCAATCAATACCTGGTTGGGTCAC
AGCTCCCATGCGAGCCCGAACCGGACGTAGCAGTGCTCACTTCCATGCTCACCG
ACCCCTCCCACATTACGGCGGAGACG
GCTAAGCGTAGGCTGGCCAGGGGATCTCCCCCCTCCTTGGCCAGCTCATCAGCT
ATCCAGCTGTCTGCGCCTTCCTTGAA
GGCAACATGCACTACCCGTCATGACTCCCCGGACGCTGACCTCATCGAGGCCAA
CCTCCTGTGGCGGCAGGAGATGGGCG
GGAACATCACCCGCGTGGAGTCAGAAAATAAGGTAGTAATTTTGGACTCTTTCG
AGCCGCTCCAAGCGGAGGAGGATGAG
AGGGAAGTATCCGTTCCGGCGGAGATCCTGCGGAGGTCCAGGAAATTCCCTCGA
GCGATGCCCATATGGGCACGCCCGGA
TTACAACCCTCCACTGTTAGAGTCCTGGAAGGACCCGGACTACGTCCCTCCAGT
GGTACACGGGTGTCCATTGCCGCCTG
CCAAGGCCCCTCCGATACCACCTCCACGGAGGAAGAGGACGGTTGTCCTGTCAG
AATCTACCGTGTCTTCTGCCTTGGCG
GAGCTCGCCACAAAGACCTTCGGCAGCTCCGAATCGTCGGCCGTCGACAGCGG
CACGGCAACGGCCTCTCCTGACCAGCC
CTCCGACGACGGCGACGCGGGATCCGACGTTGAGTCGTACTCCTCCATGCCCCC
CCTTGAGGGGGAGCCGGGGGATCCCG
ATCTCAGCGACGGGTCTTGGTCTACCGTAAGCGAGGAGGCTAGTGAGGACGTCG
TCTGCTGCTCGATGTCGTACACATGG
ACAGGCGCCCTGATCACGCCATGCGCTGCGGAGGAAACCAAGCTGCCCATCAA
TGCACTGAGCAACTCTTTGGTCCGTCA
CCACAACTTGGTCTATGCTACAACATCTCGCAGCGCAAGCCTGCGGCAGAAGAA
GGTCACCTTTGACAGACTGCAGGTCC
TGGACGACCACTACCGGGACGTGCTCAAGGAGATGAAGGCGAAGGCGTCCACA
GTTAAGGCTAAACTTCTATCCGTGGAG
GAAGCCTGTAAGCTGACGCCCCCACATTCGGCCAGATCTAAATTTGGCTATGGG
GCAAAGGACGTCCGGAACCTATCCAG
CAAGGCCGTTAACCACATCCGCTCCGTGTGGAAGGACTTGCTGGAAGACACTGA
GACACCAATtGACACTACCATCATGG
CCAAGAACGAGGTTTTCTGCGTTCAGCCTGAGAAGGGGGGTCGTAAGCCAGCTC
GTGTCATCGTGTTCCCCGACCTGGGC
GTGCGCGTGTGCGAGAAGATGGCCCTGTACGACGTGGTTAGCAAGCTCCCCCTG
GCCGTGATGGGAAGCTCCTACGGATT
CCAATACTCACCAGGACAGCGGGTTGAATTCCTCGTGCAAGCGTGGAAGTCCAA
GAAGACCCCGATGGGGTTCTCGTATG
ATACCCGCTGTTTTGACTCCACAGTCACTGAGAGCGACATCCGTACGGAGGAGG
CAATTTACCAATGTTGTGACCTGGAC
CCCCAAGCCCGCGTGGCCATCAAGTCCCTCACTGAGAGGCTTTATGTTGGGGGC
CCTCTTACCAATTCAAGGGGGGAAAA
CTGCGGCTACCGCAGGTGCCGCGCGAGCGGCGTACTGACAACTAGCTGTGGTAA
CACCCTCACTTGCTACATCAAGGCCC
GGGCAGCCTGTCGAGCCGCAGGGCTCCAGGACTGCACCATGCTCGTGTGTGGCG
ACGACTTAGTCGTTATCTGTGAAAGT
GCGGGGGTCCAGGAGGACGCGGCGAGCCTGAGAGCCTTCACGGAGGCTATGAC
CAGGTACTCCGCCCCCCCCGGGGACCC
CCCACAACCAGAATACGACTTGGAGCTTATAACATCATGCTCCTCCAACGTGTC
AGTCGCCCACGACGGCGCTGGAAAGA
GGGTCTACTACCTTACCCGTGACCCTACAACCCCCCTCGCGAGAGCCGCGTGGG
AGACAGCAAGACACACTCCAGTCAAT
TCCTGGCTAGGCAACATAATCATGTTTGCCCCCACACTGTGGGCGAGGATGATA
CTGATGACCCATTTCTTTAGCGTCCT
CATAGCCAGGGATCAGCTTGAACAGGCTCTTAACTGTGAGATCTACGGAGCCTG
CTACTCCATAGAACCACTGGATCTAC
CTCCAATCATCAAAGACTCCATGGCCTCAGCGCATTTTCACTCCACAGTTACTC
TCCAGGTGAAATCAATAGGGTGGCC
GCATGCCTCAGAAAACTTGGGGTCCCGCCCTTGCGAGCTTGGAGACACCGGGCC
CGGAGCGTCCGCGCTAGGCTTCTGTC
CAGAGGAGGCAGGGCTGCCATATGTGGCAAGTACCTCTTCAACTGGGCAGTAA
GAACAAAGCTCAAACTCACTCCAATAG
CGGCCGCTGGCCGGCTGGAGTTGTCCGGTTGGTTCACGGCTGGCTACAGCGGGG
GAGACATTTATCACAGCGTGTCTCAT
GCCCGGCCCCGCTGGTTCTGGTTTTGCCTACTCCTGCTCGCTGCAGGGGTAGGCA
TCTACCTCGTCCCCAACCGATGAAG
GTTGGGGTAAACACTCCGGCCTCTTAAGCCATTTCCTGTTTTTTTTTTTTTTTTTT
TTTTTTTTTTCTTTTTTTTTTTCT
TTCCTTTCCTTCTTTTTTTCCTTTCTTTTTCCCTTCTTTAATGGTGGCTCCATCTTA
GCCCTAGTCACGGCTAGCTGTGA
AAGGTCCGTGAGCCGCtTGACTGCAGAGAGTGCTGATACTGGCCTCTCTGCAGA
TCAaGT
BB7/H77NS5B: [SEQ ID NO: 6]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTCTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGCTATCGTGGCTGGCCACGACGGGCGTTCC
TTGCGCAGCTGTGCTCGACGTTGTCAC
TGAAGCGGGAAGGGACTGGCTGCTATTGGGCGAAGTGCCGGGGCAGGATCTCC
TGTCATCTCACCTTGCTCCTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCCT
GCTTGCCGAATATCATGGTGGAAAATGGCCGCTTTTCTGGATTCATCGACGTG
GCCGGCTGGGTGTGGCGGACCGCTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTCCACCATATTGCCGTGCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGCTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTGGACCAGGACCTCGTCG
GCTGGCAAGCGCCCCCCGGGGCGCGTT
CCTGGACACCATGCACCTGCGGCAGCTCGGACCTTTACTTGGTCACGAGGCATG
CCGATGTCATTCCGGTGCGCCGGCGG
GGCGACAGCAGGGGGAGCCTACTCTCCCCCAGGCCCGTCTCCTACTTGAAGGGC
TCTTCGGGCGGTCCACTGCTCTGCCC
CTCGGGGCACGCTGTGGGCATCTTTCGGGCTGCCGTGTGCACCCGAGGGGTTGC
GAAGGCGGTGGACTTTGTACCCGTCG
AGTCTATGGAAACCACTATGCGGTCCCCGGTCTTCACGGACAACTCGTCCCCTC
CGGCCGTACCGCAGACATTCCAGGTG
GCCCATCTACACGCCCCTACTGGTAGCGGCAAGAGCACTAAGGTGCCGGCTGCG
TATGCAGCCCAAGGGTATAAGGTGCT
TGTCCTGAACCCGTCCGTCGCCGCCACCCTAGGTTTCGGGGCGTATATGTCTAA
GGCACATGGTATCGACCCTAACATCA
GAACCGGGGTAAGGACCATCACCACGGGTGCCCCCATCACGTACTCCACCTATG
GCAAGTTTCTTGCCGACGGTGGTTGC
TCTGGGGGCGCCTATGACATCATAATATGTGATGAGTGCCACTCAACTGACTCG
ACCACTATCGTGGGCATCGGCACAGT
CCTGGACCAAGCGGAGACGGCTGGAGCGCGACTCGTCGTGCTCGCCACCGCTA
CGCCTCCGGGATCGGTCACCGTGCCAC
ATCCAAACATCGAGGAGGTGGCTCTGTCCAGCACTGGAGAAATCCCCTTTTATG
GCAAAGCCATCCCCATCGAGACCATC
AAGGGGGGGAGGCACCTCATTTTCTGCCATTCCAAGAAGAAATGTGATGAGCTC
GCCGCGAAGCTGTCCGGCCTCGGACT
CAATGCTGTAGCATATTACCGGGGCCTTGATGTATCCGTCATACCAACTAGCGG
AGACGTCATTGTCGTAGCAACGGACG
CTCTAATGACGGGCTTTACCGGCGATTTCGACTCAGTGATCGACTGCAATACAT
GTGTCACCCAGACAGTCGACTTCAGC
CTGGACCCGACCTTCACCATTGAGACGACGACCGTGCCACAAGACGCGGTGTCA
CGCTCGCAGCGGCGAGGCAGGACTGG
TAGGGGCAGGATGGGCATTTACAGGTTTGTGACTCCAGGAGAACGGCCCTCGG
GCATGTTCGATTCCTCGGTTCTGTGCG
AGTGCTATGACGCGGGCTGTGCTTGGTACGAGCTCACGCCCGCCGAGACCTCAG
TTAGGTTGCGGGCTTACCTAAACACA
CCAGGGTTGCCCGTCTGCCAGGACCATCTGGAGTTCTGGGAGAGCGTCTTTACA
GGCCTCACCCACATAGACGCCCATTT
CTTGTCCCAGACTAAGCAGGCAGGAGACAACTTCCCCTACCTGGTAGCATACCA
GGCTACGGTGTGCGCCAGGGCTCAGG
CTCCACCTCCATCGTGGGACCAAATGTGGAAGTGTCTCATACGGCTAAAGCCTA
CGCTGCACGGGCCAACGCCCCTGCTG
TATAGGCTGGGAGCCGTTCAAAACGAGGTTACTACCACACACCCCATAACCAAA
TACATCATGGCATGCATGTCGGCTGA
CCTGGAGGTCGTCACGAGCACCTGGGTGCTGGTAGGCGGAGTCCTAGCAGCTCT
GGCCGCGTATTGCCTGACAACAGGCA
GCGTGGTCATTGTGGGCAGGATCATCTTGTCCGGAAAGCCGGCCATCATTCCCG
ACAGGGAAGTCCTTTACCGGGAGTTC
GATGAGATGGAAGAGTGCGCCTCACACCTCCCTTACATCGAACAGGGAATGCA
GCTCGCCGAACAATTCAAACAGAAGGC
AATCGGGTTGCTGCAAACAGCCACCAAGCAAGCGGAGGCTGCTGCTCCCGTGGT
GGAATCCAAGTGGCGGACCCTCGAAG
CCTTCTGGGCGAAGCATATGTGGAATTCATCAGCGGGATACAATATTTAGCAG
GCTTGTCCACTCTGCCTGGCAACCCC
GCGATAGCATCACTGATGGCATTCACAGCCTCTATCACCAGCCCGCTCACCACC
CAACATACCCTCCTGTTTAACATCCT
GGGGGGATGGGTGGCCGCCCAACTTGCTCCTCCCAGCGCTGCTTCTGCTTTCGT
AGGCGCCGGCATCGCTGGAGCGGCTG
TTGGCAGCATAGGCCTTGGGAAGGTGCTTGTGGATATTTTGGCAGGTTATGGAG
CAGGGGTGGCAGGCGCGCTCGTGGCC
TTTAAGGTCATGAGCGGCGAGATGCCCTCCACCGAGGACCTGGTTAACCTACTC
CCTGCTATCCTCTCCCCTGGCGCCCT
AGTCGTCGGGGTCGTGTGCGCAGCGATACTGCGTCGGCACGTGGGCCCAGGGG
AGGGGGCTGTGCAGTGGATGAACCGGC
TGATAGCGTTCGCTTCGCGGGGTAACCACGTCTCCCCCACGCACTATGTGCCTG
AGAGCGACGCTGCAGCACGTGTCACT
CAGATCCTCTCTAGTCTTACCATCACTCAGCTGCTGAAGAGGCTTCACCAGTGG
ATCAACGAGGACTGCTCCACGCCATG
CTCCGGCTCGTGGCTAAGAGATGTTTGGGATTGGATATGCACGGTGTTGACTGA
TTTCAAGACCTGGCTCCAGTCCAAGC
TCCTGCCGCGATTGCCGGGAGTCCCCTTCTTCTCATGTCAACGTGGGTACAAGG
GAGTCTGGCGGGGCGACGGCATCATG
CAAACCACCTGCCCATGTGGAGCACAGATCACCGGACATGTGAAAAACGGTTC
CATGAGGATCGTGGGGCCTAGGACCTG
TAGTAACACGTGGCATGGAACATTCCCCATTAACGCGTACACCACGGGCCCCTG
CACGCCCTCCCCGGCGCCAAATTATT
CTAGGGCGCTGTGGCGGGTGGCTGCTGAGGAGTACGTGGAGGTTACGCGGGTG
GGGGATTTCCACTACGTGACGGGCATG
ACCACTGACAACGTAAAGTGCCCGTGTCAGGTTCCGGCCCCCGAATTCTTCACA
GAAGTGGATGGGGTGCGGTTGCACAG
GTACGCTCCAGCGTGCAAACCCCTCCTACGGGAGGAGGTCACATTCCTGGTCGG
GCTCAATCAATACCTGGTTGGGTCAC
AGCTCCCATGCGAGCCCGAACCGGACGTAGCAGTGCTCACTTCCATGCTCACCG
ACCCCTCCCACATTACGGCGGAGACG
GCTAAGCGTAGGCTGGCCAGGGGATCTCCCCCCTCCTTGGCCAGCTCATCAGCT
ATCCAGCTGTCTGCGCCTTCCTTGAA
GGCAACATGCACTACCCGTCATGACTCCCCGGACGCTGACCTCATCGAGGCCAA
CCTCCTGTGGCGGCAGGAGATGGGCG
GGAACATCACCCGCGTGGAGTCAGAAAATAAGGTAGTAATTTTGGACTCTTTCG
AGCCGCTCCAAGCGGAGGAGGATGAG
AGGGAAGTATCCGTTCCGGCGGAGATCCTGCGGAGGTCCAGGAAATTCCCTCGA
GCGATGCCCATATGGGCACGCCCGGA
TTACAACCCTCCACTGTTAGAGTCCTGGAAGGACCCGGACTACGTCCCTCCAGT
GGTACACGGGTGTCCATTGCCGCCTG
CCAAGGCCCCTCCGATACCACCTCCACGGAGGAAGAGGACGGTTGTCCTGTCAG
AATCTACCGTGTCTTCTGCCTTGGCG
GAGCTCGCCACAAAGACCTTCGGCAGCTCCGAATCGTCGGCCGTCGACAGCGG
CACGGCAACGGCCTCTCCTGACCAGCC
CTCCGACGACGGCGACGCGGGATCCGACGTTGAGTCGTAGTCCTCCATGCCCCC
CCTTGAGGGGGAGCCGGGGGATCCCG
ATCTCAGCGACGGGTCTTGGTCTACCGTAAGCGAGGAGGCTAGTGAGGACGTCG
TCTGCTGCTCGATGTCCTACACATGG
ACAGGCGCCCTGATCACCCCGTGCGCTGCGGAAGAACAAAAACTGCCCATCAA
CGCACTGAGCAACTCGTTGCTACGCCA
TCACAATCTGGTGTATTCCACCACTTCACGCAGTGCTTGCCAAAGGCAGAAGAA
AGTCACATTTGACAGACTGCAAGTTC
TGGACAGCCATTACCAGGACGTGCTCAAGGAGGTCAAAGCAGCGGCGTCAAAA
GTGAAGGCTAACTTGCTATCCGTAGAG
GAAGCTTGCAGCCTGACGCCCCCACATTCAGCCAAATCCAAGTTTGGCTATGGG
GCAAAAGACGTCCGTTGCCATGCCAG
AAAGGCCGTAGCCCACATCAACTCCGTGTGGAAAGACCTTCTGGAAGACAGTGT
AACACCAATAGACACTACCATCATGG
CCAAGAACGAGGTTTTCTGCGTTCAGCCTGAGAAGGGGGGTCGTAAGCCAGCTC
GTCTCATCGTGTTCCCCGACCTGGGC
GTGCGCGTGTGCGAGAAGATGGCCCTGTACGACGTGGTTAGCAAGCTCCCCCTG
GCCGTGATGGGAAGCTCCTACGGATT
CCAATACTCACCAGGACAGCGGGTTGAATTCCTCGTGCAAGCGTGGAAGTCCAA
GAAGACCCCGATGGGGTTCTCGTATG
ATACCCGCTGTTTTGACTCCACAGTCACTGAGAGCGACATCCGTACGGAGGAGG
CAATTTACCAATGTTGTGACCTGGAC
CCCCAAGCCCGCGTGGCCATCAAGTCCCTCACTGAGAGGCTTTATGTTGGGGGC
CCTCTTACCAATTCAAGGGGGGAAAA
CTGCGGCTACCGCAGGTGCCGCGCGAGCGGCGTACTGACAACTAGCTGTGGTAA
CACCCTCACTTGCTACATCAAGGCCC
GGGCAGCCTGTCGAGCCGCAGGGCTCCAGGACTGCACCATGCTCGTGTGTGGCG
ACGACTTAGTCGTTATCTGTGAAAGT
GCGGGGGTCCAGGAGGACGCGGCGAGCCTGAGAGCCTTCACGGAGGCTATGAC
CAGGTAGTCCtCCCCCCCCGGGGACCC
CCCACAACCAGAATACGACTTGGAGCTTATAACATCATGCTCGTCCAACGTGTC
AGTCGCCCACGACGGCGCTGGAAAGA
GGGTCTACTACCTTACCCGTGACCCTACAACCCCCCTCGCGAGAGCCGCGTGGG
AGACAGCAAGACACACTCCAGTCAAT
TCCTGGCTAGGCAACATAATCATGTTTGCCCCCACACTGTGGGCGAGGATGATA
CTGATGACCCATTTCTTTAGCGTCCT
CATAGCCAGGGATCAGCTTGAACAGGCTCTTAACTGTGAGATCTACGGAGCCTG
CTACTCCATAGAACCACTGGATCTAC
CTCCAATCATTCAAAGACTCCATGGCCTCAGCGCATTTTCACTCCACAGTTACTC
TCCAGGTGAAATCAATAGGGTGGCC
GCATGCCTCAGAAAACTTGGGGTCCCGCCCTTGCGAGCTTGGAGACACCGGGCC
CGGAGCGTCCGCGCTAGGCTTCTGTC
CAGAGGAGGCAGGGCTGCCATATGTGGCAAGTACCTCTTCAACTGGGCAGTAA
GAACAAAGCTCAAACTCACTCCAATAG
CGGCCGCTGGCCGGCTGGACTTGTCCGGTTGGTTCACGGCTGGCTACAGCGGGG
GAGACATTTATCACAGCGTGTCTCAT
GCCCGGCCCCGCTGGTTCTGGTTTTGCGTACTCCTGCTCGCTGCAGGGGTAGGCA
TCTACCTCCTCCCCAACCGATGAAG
GTTtacGTAAACACTCCAGGCCAATAGGCCATCCTGTTTTTTTCCCTTTTTTTTTTT
CTTTTTTTTTTTTTTTTTTTTTT
TTTTTTTTTTTTCTCCTTTTTTTTTCCTCTTTTTTTCCTTTTCTTTCCTTTGGTGGCT
CCATCTTAGCCCTAGTCACGGC
TAGCTGTGAAAGGTCCGTGAGCCGCTTGAGTGCAGAGAGTGCTGATACTGGCCT
CTCTGCAGATCAAGT
HCV 1a replicon: [SEQ ID NO: 7]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTCTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGCTATCGTGGCTGGCCACGACGGGCGTTCC
TTGCGCAGCTGTGCTCGACGTTGTCAC
TGAAGCGGGAAGGGACTGGGTGCTATTGGGCGAAGTGCCGGGGCAGGATCTCC
TGTCATCTCACCTTGCTCGTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCCT
GCTTGCCGAATATCATGGTGGAAAATGGCCGCTTTTCTGGATTCATCGAGTGTG
GCCGGCTGGGTGTGGCGGACCGCTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTGTGGAAAGAGTCAAATGGCTCTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCGTACTCCCAACAGACGCGAGGCC
TACTTGGCTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTGGACCAAGACCTTGTGGG
CTGGCCCGCTCCTCAAGGTTCCCGCT
CATTGACACCCTGTACCTGCGGCTCCTCGGACCTTTACCTGGTCACGAGGCACG
CCGATGTCATTCCCGTGCGCCGGCGA
GGTGATAGCAGGGGTAGCCTGCTTTCGCCCCGGCCCATTTCCTACTTGAAAGGC
TCCTCGGGGGGTCCGCTGTTGTGCCC
CGCGGGACACGCCGTGGGCCTATTCAGGGCCGCGGTGTGCACCCGTGGAGTGG
CTAAAGCGGTGGACTTTATCCCTGTGG
AGAACCTAGGGACAACCATGAGATCCCCGGTGTTCACGGACAACTCCTCTCCAC
CAGCAGTGCCCCAGAGCTTCCAGGTG
GCCCACCTGCATGCTCCCACCGGCAGCGGTAAGAGCACCAAGGTCCCGGCTGC
GTACGCAGCCCAGGGCTACAAGGTGTT
GGTGCTCAACCCCTCTGTTGCTGCAACGCTGGGCTTTGGTGCTTACATGTCCAAG
GCCCATGGGGTTGATCCTAATATCA
GGACCGGGGTGAGAACAATTACCACTGGCAGCCCCATCACGTACTCCACCTACG
GCAAGTTCCTTGCCGACGGCGGGTGC
TCAGGAGGTGCTTATGACATAATAATTTGTGACGAGTGCCACTCCACGGATGCC
ACATCCATCTTGGGCATCGGCACTGT
CCTTGACCAAGCAGAGACTGCGGGGGCGAGACTGGTTGTGCTCGCCACTGCTAC
CCCTCCGGGCTCCGTCACTGTGcCCC
ATCCTAACATCGAGGAGGTTGCTCTGTCCACCACCGGAGAGATCCCCTTTTACG
GCAAGGCTATCCCCCTCGAGGTGATC
AAGGGGGGAAGACATCTCATCTTCTGCCACTCAAAGAAGAAGTGCGACGAGCT
CGCCGCGAAGCTGGTCGCATTGGGCAT
CAATGCCGTGGCCTACTACCGCGGTCTTGACGTGTCTGTCATCCCGACCAGCGG
CGATGTTGTCGTCGTGTCGACCGATG
CTCTCATGACTGGCTTTACCGGCGACTTCGACTGTGTGATAGACTGCAACACGT
GTGTCACTCAGACAGTCGATTTCAGC
CTTGACCCTACCTTTACCATTGAGACAACCACGCTCCCCCAGGATGCTGTGTCCA
GGACTCAACGCCGGGGCAGGACTGG
CAGGGGGAAGCCAGGCATCTATAGATTTGTGGCACCGGGGGAGCGCCCCTCCG
GCATGTTCGACTCGTCCGTCCTCTGTG
AGTGCTATGACGCGGGCTGTGCTTGGTATGAGCTCACGCCCGCCGAGACTACAG
TTAGGCTACGAGCGTACATGAACACC
CCGGGGCTTCCCGTGTGCCAGGACCATCTTGAATTTTGGGAGGGCGTCTTTACG
GGCCTCACTCATATAGATGCCCACTT
TTTATCCCAGACAAAGCAGAGTGGGGAGAACTTTCCTTACCTGGTAGCGTACCA
AGCCACCGTGTGCGCTAGGGCTCAAG
CCCCTCCCCCATCGTGGGACCAGATGTGGGAGTGTTTGATCCGCCTTAAACCCAC
CCTCCATGGGCCAACACCCCTGCTA
TACAGACTGGGCGCTGTTCAGAATGAAGTCACCCTGACGCACCCAATCACCAAA
TACATCATGACATGCATGTCGGCCGA
CCTGGAGGTCGTCACGAGCACCTGGGTGCTCGTTGGCGGCGTCCTGGCTGCTCT
GGCCGCGTATTGCCTGTCAACAGGCT
GCGTGGTCATAGTGGGCAGGATCGTCTTGTCCGGGAAGCCGGCAATTATACCTG
ACAGGGAGGTTCTCTACCAGGAGTTC
GATGAGATGGAAGAGTGCTCTCAGCACTTACCGTACATCGAGCAAGGGATGAT
GCTCGCTGAGCAGTTCAAGCAGAAGGC
CCTCGGCCTCCTGCAGACCGCGTCCCGCCATGCAGAGGTTATCACCCCTGCTGT
CCAGACCAACTGGCAGAAACTCGAGG
TCTTTTGGGCGAAGCACATGTGGAATTTCATCAGTGGGATACAATACTTGGCGG
GCCTGTCAACGCTGCCTGGTAACCCC
GCCATTGCTTCATTGATGGGCTTTTACAGCTGCCGTCACCAGCCCACTAACCACTG
GCCAAACCCTCCTCTTCAACATATT
GGGGGGGTGGGTGGCTGCCCAGCTCGCCGCCCCCGGTGCCGCTACTGCCTTTGT
GGGTGCTGGCCTAGCTGGCGCCGCCA
TCGGCAGCGTTGGACTGGGGAAGGTCCTCGTGGACATTCTTGCAGGGTATGGCG
CGGGCGTGGCGGGAGCTCTTGTAGCA
TTCAAGATCATGAGCGGTGAGGTCCCCTCCACGGAGGACCTGGTCAATCTGGTG
CCCGCCATCCTCTCGCCTGGAGCCCT
TGTAGTCGGTGTGGTCTGCGCAGCAATACTGCGCCGGCACGTTGGCCCGGGCGA
GGGGGCAGTGCAATGGATGAACCGGC
TAATAGCCTTCGCCTCCCGGGGGAACCATGTTTCCCCCACGCACTACGTGCCGG
AGAGCGATGCAGCCGCCCGCGTCACT
GCCATACTCAGCAGCCTCACTGTAACCCAGCTCCTGAGGCGACTGCATCAGTGG
ATAAGCTCGGAGTGTACCACTCCATG
CTCCGGTTCCTGGCTAAGGGACATGTGGGACTGGATATGCGtGGTGCTGAGCGA
CTTTAAGACCTGGCTGAAAGCCAAGC
TCATGCCACAACTGCCTGGGATTCCCTTTGTGTCCTGCCAGCGCGGGTATAGGG
GGGTCTGGCGAGGAGACGGCATTATG
CACACTCGCTGCCACTGTGGAGCTGAGATCACTGGACATGTCAAAAACGGGAC
GATGAGGATCGTCGGTCCTAGGACCTG
CAGGAACATGTGGAGTGGGACGTTCCCCATTAACGCCTACACCACGGGCCCCTG
TACTCCCCTTCCTGCGCCGAACTATA
AGTTCGCGCTGTGGAGGGTGTCTGCAGAGGAATACGTGGAGATAAGGCGGGTG
GGGGACTTCCACTACGTATCGGGTATG
AGTACTGACAATCTAAATGCCCGTGCCAGATCCCATCGCCCGAATTcTTCACAG
AATTGGACGGGGTGCGCCTACACAG
GTTTGCGCCCCCTTGCAAGCCCTTGCTGCGGGAGGAGGTATCATTCAGAGTAGG
ACTCCACGAGTACCCGGTGGGGTCGC
AATTACCTTGCGAGCCCGAACCGGACGTAGCCGTGTTGACGTCCATGCTCAGTG
ATCCCTCCCATATAACAGCAGAGGCG
GCCGGGAGAAGGTTGGCGAGAGGGTCACCCCCTTCTATGGCCAGCTCCTCGGCT
AtCCAGCTGTCCGGTCCATCTCTCAA
GGCAACTTGCACCGCCAACCATGACTCCCCTGACGCCGAGCTCATAGAGGCTAA
CCTCCTGTGGAGGCAGGAGATGGGCG
GCAACATCACCAGGGTTGAGTCAGAGAACAAAGTGGTGATTCTGGACTCCTTCG
ATCCGCTTGTGGCAGAGGAGGATGAG
CGGGAGGTCTCCGTACCTGCAGAAATTCTGCGGAAGTCTCGGAGATTCGCCCGG
GCCCTGCCCGTCTGGGCGCGGCCGGA
CTACAACCCCCCGCTAGTAGAGACGTGGAAAAAGCCTGACTACGAACCACCTGT
GGTCCATGGCTGCCCGCTACCACCTC
CACGGTCCCCTCCTGTGCCTCCGCCTCGGAAAAAGCGTACGGTGGTCCTCACCG
AATCAACCCTATCTACTGCCTTGGCC
GAGCTTGCCACCAAAAGTTTTGGCAGCTCCTCAACTTCCGGCATTACGGGCGAC
AATACGACAACATCCTCTGAGCCCGC
CCCTTCTGGCTGCCCCCCCGACTCCGACGTTGAGTCCTATTCTTCCATGCCCCCC
CTGGAGGGGGAGCCTGGGGATCCGG
ATGTCAGCGACGGGTCATGGTCGACGGTCAGTAGTGGGGCCGACACGGAAGAT
GTCGTGTGCTGCTCAATGTCTTATTCC
TGGACAGGCGCACTCGTCACCCCGTGCGCTGCGGAAGAACAAAAACTGCCCAT
CAACGCACTGAGCAACTCGTTGCTACG
CCATCACAATCTGGTGTATTCCACCACTTCACGCAGTGCTTGCCAAAGGCAGAA
GAAAGTCACATTTGACAGACTGCAAG
TTCTGGACAGCCATTACCAGGACGTGCTCAAGGAGGTCAAAGCAGCGGCGTCA
AAAGTGAAGGCTAACTTGCTATCCGTA
GAGGAAGCTTGCAGCCTGACGCCCCCACATTCAGCCAAATCCAAGTTTGGCTAT
GGGGCAAAAGACGTCCGTTGCCATGC
CAGAAAGGCCGTAGCCCACATCAACTCCGTGTGGAAAGACCTTCTGGAAGACA
GTGTAACACCAATtGACACTACCATCA
TGGCCAAGAACGAGGTTTTCTGCGTTCAGCCTGAGAAGGGGGGTCGTAAGCCA
GCTCGTCTCATCGTGTTCCCCGACCTG
GGCGTGCGCGTGTGCGAGAAGATGGCCCTGTACGACGTGaTTAGCAAGCTCCCC
GTGGCCGTGATGGGAAGCTCCTACGG
ATTCCAATACTCACCAGGACAGCGGGTTGAATTCCTCGTGCAAGCGTGGAAGTC
CAAGAAGACCCCGATGGGGTTCTCGT
ATGATACCCGCTGTTTTGACTCCACAGTCACTGAGAGCGACATCCGTACGGAGG
AGGCAATTTACCAATGTTGTGACCTG
GACCCCCAAGCCCGCGTGGCCATCAAGTCCCTCACTGAGAGGCTTTATGTTGGG
GGCCCTCTTACCAATTCAAGGGGGGA
AAACTGCGGCTACCGCAGGTGCCGCGCGAGCGGCGTACTGACAACTAGCTGTG
GTAACACCCTCACTTGCTACATCAAGG
CCCGGGCAGCCTGTCGAGCCGCAGGGCTCCAGGACTGCACCATGCTCGTGTGTG
GaGACGACTTAGTCGTTATCTGTGAA
AGTGCGGGGGTCCAGGAGGACGCGGCGAGCCTGAGAGCCTTCACGGAGGCTAT
GACCAGGTACTCCGCCCCCCCCGGGGA
CCCCCCACAACCAGAATACGACTTGGAGCTTATAACATCATGCTCCTCCAACGT
GTCAGTCGCCCACGACGGCGCTGGAA
AGAGGGTCTACTACCTTACCCGTGACCCTACAACCCCCCTCGAGAGAGCCGCGT
GGGAGACAGCAAGACACACTCCAGTC
AATTCCTGGCTAGGCAACATAATCATGTTTGCCCCCACACTGTGGGCGAGGATG
ATACTGATGACCCATTTCTTTAGCGT
CCTCATAGCCAGGGATCAGCTTGAACAGGCTCTTAACTGTGAGATCTACGGAGC
CTGCTACTCCATAGAACCACTaGATC
TACCTCCAATCATTCAAAGACTCCATGGCCTCAGCGCATTTTCACTCCACAGTTA
CTCTCCAGGTGAAATCAATAGGGTG
GCCGCATGCGTCAGAAAACTTGGGGTCCCGCCCTTGCGAGCTTGGAGACACCGG
GCCCGGAGCGTCCGCGCTAGGCTTCT
GTCCAGAGGAGGCAGGGCTGCCATATGTGGCAAGTACCTCTTCAACTGGGCAGT
AAGAACAAAGCTCAAACTCACTCCAA
TAGCGGCCGCTGGCCGGCTGGACTTGTCCGGTTGGTTCACGGCTGGCTACAGCG
GGGGAGACATTTATCACAGCGTGTCT
CATGCCCGGCCCCGCTGGTTCTGGTTTTGCCTACTCCTGCTCGCTGCAGGGGTAG
GCATCTACCTCCTCCCCAACCGATG
AAGGTTGGGGTAAACACTCCGGCCTCTTAAGCCATTTCCTGTTTTTTTTTTTTTTT
TTTTTTTTTTTTTCTTTTTTTTTT
TCTTTCCTTTCCTTCTTTTTTTCCTTTCTTTTTCCCTTCTTTAATGGTGGCTCCATC
TTAGCCCTAGTCACGGCTAGCTG
TGAAAGGTCCGTGAGCCGCtTGACTGCAGAGAGTGCTGATACTGGCCTCTGTGC
AGATCAaGT
(B) Sequences from HCV J4 Replicons
J4 M/S: [SEQ ID NO: 8]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTCTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGCTATCGTGGCTGGCCACGACGGGCGTTCC
TTGCGCAGCTGTGCTCGACGTTGTCAC
TGAAGCGGGAAGGGACTGGCTGCTATTGGGCGAAGTGCCGGGGCAGGATCTCC
TGTCATCTCACCTTGCTCCTGCCGAGA
AAGTATCCATCATGGCTGATGCAATGCGGCGGCTGCATACGCTTGATCCGGGTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCGT
GCTTGCCGAATATCATGGTGGAAAATGGCCGCTTTTCTGGATTCATCGAGTGTG
GCCGGCTGGGTGTGGCGGACCGCTAT
CAGGACATAGCGTfGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGCTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTAGACGTGGACCTCGTCGG
CTGGCAGGCGCCCCCCGGGGCGCGCT
CCATGACACCATGCAGCTGTGGCAGCTCGGACCTTTACTTGGTCACGAGACATG
CTGATGTCATTCCGGTGCGCCGGCGA
GGCGACAGCAGGGGAAGTCTACTCTCCCCCAGGCCCGTCTCCTACCTGAAAGGC
TCCTCGGGTGGTCCATTGCTTTGCCC
TTCGGGGCACGTCGTGGGCGTCTTCCGGGCTGCTGTGTGCACCCGGGGGGTCGC
GAAGGCGGTGGACTTCATACCCGTTG
AGTCTATGGAAACTACCATGCGGTCTCCGGTCTTCACAGACAACTCAACCCCCC
CGGCTGTACCGCAGACATTCCAAGTG
GCACATCTGCACGCTCCTACTGGCAGCGGCAAGAGCACCAAAGTGCCGGCTGC
GTATGCAGCCCAAGGGTACAAGGTGCT
CGTCCTGAACCCGTCCGTTGCCGCCACCTTAGGGTTTGGGGCGTATATGTCCAA
GGCACACGGTATCGACCCTAACATCA
GAACTGGGGTAAGGACCATTACCACGGGCGGCTCCATTACGTACTCCACCTATG
GCAAGTTCCTTGCCGACGGTGGCTGT
TCTGGGGGCGCCTATGACATCATAATATGTGATGAGTGCCACTCAACTGACTCG
ACTACCATCTTGGGCATCGGCACAGT
CCTGGACCAAGCGGAGACGGCTGGAGCGCGGCTCGTCGTGCTCGCCACCGCTA
CACCTCCGGGATCGGTTACCGTGCCAC
ACCCCAATATCGAGGAAATAGGCCTGTCCAACAATGGAGAGATCCCCTTCTATG
GCAAAGCCATCCCCATTGAGGCCATC
AAGGGGGGGAGGCATCTCATTTTCTGCCATTCCAAGAAGAAATGTGACGAGCTC
GCCGCAAAGCTGACAGGCCTCGGACT
GAACGCTGTAGCATATTACCGGGGCCTTGATGTGTCCGTCATACCGCCTATCGG
AGACGTCGTTGTCGTGGCAACAGACG
CTCTAATGACGGGTTTCACCGGCGATTTTGACTCAGTGATCGACTGCAATACAT
GTGTCACCCAGACAGTCGACTTCAGC
TTGGATCCCACCTTCACCATTGAGACGACGACCGTGCCCCAAGACGCGGTGTCG
CGCTCGCAACGGCGAGGTAGAACTGG
CAGGGGTAGGAGTGGCATCTACAGGTTTGTGACTCCAGGAGAACGGCCCTCGG
GCATGTTCGATTCTTCGGTCCTGTGTG
AGTGCTATGACGCGGGCTGTGCTTGGTATGAGCTCACGCCCGCTGAGACCTCGG
TTAGGTTGCGGGCTTACGTAAATACA
CCAGGGTTGCCCGTCTGCCAGGACCATCTGGAGTTCTGGGAGAGCGTCTTCACA
GGCCTCACCCACATAGATGCCCACTT
CCTGTCCCAGACTAAACAGGCAGGAGACAACTTTCCTTACCTGGTGGCATATCA
AGCTACAGTGTGCGCCAGGGCTCAAG
CTCCACCTCCATCGTGGGACCAAATGTGGAAGTGTCTCATACGGCTGAAACCTA
CACTGCACGGGCCAACACCCCTGCTG
TATAGGCTAGGAGCCGTCCAAAATGAGGTCATCGTCACACACCCCATAACTAAA
TACATCATGGCATGCATGTCGGCTGA
CCTGGAGGTCGTCACTAGCACCTGGGTGCTGGTAGGCGGAGTCCTTGCAGCTTT
GGCCGCATACTGCCTGACGACAGGCA
GTGTGGTCATTGTGGGCAGGATCATCTTGTCCGGGAAGCCAGCTGTCGTTCCCG
ACAGGGAAGTCCTCTACCAGGAGTTC
GATGAGATGGAAGAGTGTGCCTCACAACTTCCTTACATCGAGCAGGGAATGCA
GCTCGCCGAGCAATTCAAGCAAAAGGC
GGTCGGGTTGTTGCAAACGGCCACCAAGCAAGCGGAGGCTGCTGCTCCCGTGGT
GGAGTCCAAGTGGCGAGCCCTTGAGA
CCTTCTGGGCGAAGCACATGTGGAATTTCATCAGCGGAATACAGTACCTAGCAG
GCTTATCCACTCTGCCTGGAAACCCC
GCGATAGCATCATTGATGGCATTTACAGCTTCTATCACTAGCCCGCTCACCACCC
AAAACACCCTCCTGTTTAACATCTT
GGGGGGATGGGTGGCTGCCCAACTCGCTCCTCCCAGCGCTGCGTCAGCTTTCGT
GGGCGCCGGCATCGCCGGAGCGGCTG
TTGGCAGCATAGGCCTTGGGAAGGTGCTCGTGGACATCTTGGCGGGCTATGGGG
CAGGGGTAGCCGGCGCACTCGTGGCC
TTTAAGGTCATGAGCGGCGAGGTGCCCTCCACCGAGGACCTGGTCAACTTACTC
CGTGCCATCCTCTCTCCTGGTGCCCT
GGTCGTCGGGGTCGTGTGCGCAGCAATACTGCGTCGGCACGTGGGCCCGGGAG
AGGGGGCTGTGCAGTGGATGAACCGGC
TGATAGCGTTCGCTTCGCGGGGTAACCACGTCTCCCCTACGCACTATGTGCCTG
AGAGCGACGCTGCAGCACGTGTCACT
CAGATCCTCTCTAGCCTTACCATCACTCAACTGCTGAAGCGGCTCCACCAGTGG
ATTAATGAGGACTGCTCTACGCCATG
CTCCGGCTCGTGGCTAAGGGATGTTTGGGATTGGATATGCACGGTGTTGACTGA
CTTCAAGACCTGGCTCCAGTCCAAAC
TCCTGCCGCGGTTACCGGGAGTCCCTTTCCTGTCATGCCAACGCGGGTACAAGG
GAGTCTGGCGGGGGGACGGCATCATG
CAAACCACCTGCCCATGCGGAGCACAGATCGCCGGACATGTCAAAAACGGTTC
CATGAGGATCGTAGGGCCTAGAACCTG
CAGCAACACGTGGCACGGAACGTTCCCCATCAACGCATACACCACGGGACCTT
GCACACCCTCCCCGGCGCCCAACTATT
CCAGGGCGCTATGGCGGGTGGGTGCTGAGGAGTACGTGGAGGTTACGCGTGTG
GGGGATTTCCACTACGTGACGGGCATG
ACCACTGACAACGTAAAGTGCCCATGCCAGGTTCCGGCCCCCGAATTCTTCACG
GAGGTGGATGGAGTGCGGTTGCACAG
GTACGCTCCGGCGTGCAAACCTCTTCTACGGGAGGACGTCACGTTCCAGGTCGG
GCTCAACCAATACTTGGTCGGGTCGC
AGCTCCCATGCGAGCCCGAACCGGACGTAACAGTGCTTACTTCCATGCTCACCG
ATCCCTCCCACATTACAGCAGAGACG
GCTAAGCGTAGGCTGGCTAGAGGGTGTCCCCCCTCTTTAGCCAGCTCATCAGCT
AtCCAGTTGTCTGCGCCTTCTTTGAA
GGCGACATGCACTACCCACCATGACTCCCCGGACGCTGACCTCATCGAGGCCAA
CCTCTTGTGGCGGCAGGAGATGGGCG
GAAACATCACTCGCGTGGAGTCAGAGAATAAGGTAGTAATTCTGGACTCTTTCG
AACCGCTTTCACGCGGAGGGGGATGAG
AGGGAGATATCCGTCGCGGCGGAGATCCTGCGAAAATCCAGGAAGUCCCCTC
AGCGTTGCCCATATGGGCACGCCCGGA
CTACAATCCTCCACTGCTAGAGTCCTGGAAGGACCCGGACTACGTCCCTCCGGT
GGTACACGGATGCCCATTGCCACCTA
CCAAGGCTCCTCCAATACCACCTCCACGGAGAAAGAGGACGGTTGTCCTGACAG
AATCCAATGTGTCTTCTGCGTTGGCG
GAGCTCGCCACTAAGACCTTCGGTAGCTCCGGATCGTCGGCCGTTGATAGCGGC
ACGGCGACCGCCCTTCCTGACGTGGC
CTCCGACGACGGTGACAAAGGATCCGACGTTGAGTCGTACTCCTCCATGCCCCC
CCTTGAAGGGGAGCCGGGGGACCCCG
ATCTCAGCGACGGGTCTTGGTCTACCGTGAGTGAGGAGGCTAGTGAGGATGTCG
TCTGGTGCTCAATGTCCTATACGTGG
ACAGGCGCCCTGATCACGCCATGCGCTGCGGAGGAAAGTAAGCTGCCCATCAA
CCCGTTGAGCAACTCTTTGCTGCGTCA
CCACAACATGGTCTACGCCACAACATCCCGCAGCGCAAGCCTCCGGCAGAAGA
AGGTCACCTTTGACAGATTGCAAGTCC
TGGATGATCATTACCGGGACGTACTCAAGGAGATGAAGGCGAAGGCGTCCACA
GTTAAGGCTAAGCTTCTATCTATAGAG
GAGGCCTGCAAGCTGACGCCCCCACATTCGGCCAAATCCAAATTTGGCTATGGG
GCAAAGGACGTCCGGAACCTATCCAG
CAGGGCCGTTAACCACATCCGCTCCGTGTGGGAGGACTTGCTGGAAGACACTGA
AACACCAATTGACACCACCATCATGG
CAAAAAATGAGGTTTTCTGCGTCCAACCAGAGAAGGGGGGCCGCAAGCCAGCT
CGCCTTATCGTATTCCCAGATTTGGGG
GTTCGTGTGTGCGAGAAAATGGCCCTTTACGATGTGGTGTCCACCCTCCTCAG
GCCGTGATGGGCTCTTCATACGGATT
CCAATACTCTCCTGGACAGCGGGTCGAGTTCCTGGTGAATGCCTGGAAAGCGAA
GAAATGCCCTATGGGCTTCGCATATG
ACACCCGCTGTTTTGACTCAACGGTCACTGAGAATGACATCCGTGTTGAGGAGT
CAATCTACCAATGTTGTGACTTGGCC
CCCGAAGCCAGACAGGCCATAAGGTCGCTCACAGAGCGGCTTTACATCGGGGG
CCCCCTGACTAATTCTAAAGGGCAGAA
CTGCGGCTATCGCCGGTGCCGCGCGAGCGGTGTACTGACGACCAGCTGCGGTAA
TACCCTCACATGTTACTTGAAGGCCG
CTGCGGCCTGTCGAGCTGCGAAGCTCCAGGACTGCACGATGCTCGTATGCGGAG
ACGACCTTGTCGTTATCTGTGAAAGC
GCGGGGACCCAAGAGGACGAGGCGAGCCTACGGGCCTTCACGGAGGCTATGAC
TAGATACTCTGCCCCCCCTGGGGACCC
GCCCAAACCAGAATACGACTTGGAGTTGATAACATCATGCTCCTCCAATGTGTC
AGTCGCGCACGATGCATCTGGCAAAA
GGGTGTACTATCTCACCCGTGACCCCACCACCCCCCTTGCGCGGGCTGCGTGGG
AGACAGCTAGACACACTCCAGTCAAT
TCGTGGCTAGGCAACATCATCATGTATGCGCCCACCTTGTGGGCAAGGATGATC
CTGATGACTCATTTCTTCTCCATCCT
TCTAGCTCAGGAACAACTTGAAAAAGCCCTAGATTGTCAGATCTACGGGGCCTG
TTACTCCATTGAGCCACTTGACCTAC
CTCAGATCATTCAACGACTCCATGGCCTTAGCGCATTTTCACTCCATAGTTACTC
TCCAGGTGAGATCAATAGGGTGGCT
TCATGCCTCAGGAAACTTGGGGTACCGCCCTTGCGAGTGTGGAGACATCGGGCC
AGAAGTGTCCGCGCTAGGCTACTGTC
CCAGGGGGGGAGGGCTGCCACTTGTGGCAAGTACCTCTTCAACTGGGCAGTAA
GGACCAAGCTCAAACTCACTCCAATCC
CGGCTGCGTCCCAGTTGGATTTATCCAGCTGGTTCGTTGCTGGTTACAGCGGGG
GAGACATATATCACAGCCTGTCTCGT
GCCCGACCCCGCTGGTTCATGTGGTGCCTACTCGTACTTTCTGTAGGGGTAGGCA
TGTATCTACTCCCCAACCGATGAAC
GGGGACCTAAACACTCCAGGCCAATAGGCCATCCTGTTTTTTTCCCTTTTTTTTT
TTCTTTTTTTTTTTTTTTTTTTTTT
TTTTTTTTTTTTCTCCTTTTTTTTTCCTCTTTTTTTCCTTTTCTTTCCTTTGGTGGCT
CCATCTTAGCCCTAGTCACGGC
TAGCTGTGAAAGGTCCGTGAGCCGCTTGACTGCAGAGAGTGCTGATACTGGCCT
CTCTGCAGATCAAGT
J4 B/R1: [SEQ ID NO: 9]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCGTCCAGGACCCCCCCTCCCGGGA
GAGCCATAGTGGTGTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGGTATCGTGGCTGGCCACGACGGGCGTTCC
TTGCGCAGCTGTGCTCGACGTTGTCAC
TGAAGCGGGAAGGGACTGGCTGCTATTGGGCGAAGTGCCGGGGCAGGATCTCC
TGTCATCTCACCTTGCTCCTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCCT
GCTTGCCGAATATCATGGTGGAAAATGGCCGCTTTTCTGGATTCATCGACTGTG
GCCGGCTGGGTGTGGCGGACCGCTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTGTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCCTCAAGCGTATTCAACAAGGGGCTGA
AGGATGCCCAGAAGGTACCCCATTGT
ATGGGATCTGATGTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCGTATTAC
GGCCTACTCCCAACAGACGCGAGGCC
TACTTGGCTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTGGACCAGGACCTCGTCG
GCTGGCAAGCGCCCCCCGGGGCGCGTT
CCTTGACACCATGCACCTGCGGCAGCTCGGACCTTTACTTGGTCACGAGGCATG
CCGATGTCATTCCGGTGCGCCGGCGG
GGCGACAGCAGGGGGAGCCTACTCTCCCCCAGGCCCGTCTCCTACTTGAAGGGC
TCTTCGGGCGGTCCACTGCTGTGCCC
CTCGGGGCACGCTGTGGGCATCTTTCGGGCTGCCGTGTGCACCCGAGGGGTTGC
GAAGGCGGTGGACTTTGTACCCGTCG
AGTCTATGGAAACCACTATGCGGTCCCCGGTCTTCACGGACAACTCGTCCCCTC
CGGCCGTACCGCAGACATTCCAGGTG
GCCCATCTACACGCCCCTACTGGTAGCGGCAAGAGCACTAAGGTGCCGGCTGCG
TATGCAGCCCAAGGGTATAAGGTGCT
TGTCGTGAACCCGTCCGTCGCCGCCACCCTAGGTTTCGGGGCGTATATGTCTAA
GGCACATGGTATCGACCCTAACATCA
GAACCGGGGTAAGGACCATCACCACGGGTGCCCCCATCACGTACTCCACCTATG
GCAAGTTTCTTGCCGACGGTGGTTGC
TCTGGGGGCGCCTATGACATCATAATATGTGATGAGTGCCACTCAACTGACTCG
ACCACTATCCTGGGCATCGGCACAGT
CCTGGACCAAGCGGAGACGGCTGGAGCGCGACTCGTCGTGCTCGCCACCGCTA
CGCCTCCGGGATCGGTCACCGTGCCAC
ATCCAAACATCGAGGAGGTGGCTCTGTCCAGCACTGGAGAAATCCCCTTTTATG
GCAAAGCCATCCCCATCGAGACCATC
AAGGGGGGGAGGCACCTCATTTTCTGCCATTCCAAGAAGAAATGTGATGAGCTC
GCCGCGAAGCTGTCCGGCCTCGGACT
CAATGCTGTAGCATATTACCGGGGCCTTGATGTATCCGTCATACCAACTAGCGG
AGACGTCATTGTCGTAGCAACGGACG
CTCTAATGACGGGCTTTACCGGCGATTTCGACTCAGTGATCGACTGCAATACAT
GTGTCACCCAGACAGTCGACTTCAGC
CTGGACCCGACCTTCACCATTGAGACGACGACCGTGCCACAAGACGCGGTGTCA
CGCTCGCAGCGGCGAGGCAGGACTGG
TAGGGGCAGGATGGGCATTTACAGGTTTGTGACTCCAGGAGAACGGCCCTCGG
GCATGTTCGATTCCTCGGTTCTGTGCG
AGTGCTATGACGCGGGCTGTGCTTGGTACGAGCTCACGCCCGCCGAGACCTCAG
TTAGGTTGCGGGCTTACCTAAACACA
CCAGGGTTGCCCGTCTGCCAGGACCATCTGGAGTTGTGGGAGAGCGTCTTTACA
GGCCTCACCCACATAGACGCCCATTT
CTTGTCCCAGACTAAGCAGGCAGGAGACAACTTCCCCTACCTGGTAGCATACCA
GGCTACGGTGTGCGCCAGGGCTCAGG
CTCCACCTCCATCGTGGGACCAAATGTGGAAGTGTCTCATACGGCTAAAGCCTA
CGCTGCACGGGCCAACGCCCCTGCTG
TATAGGCTGGGAGCCGTTCAAAACGAGGTTACTACCACACACCCCATAACCAAA
TACATCATGGCATGCATGTCGGCTGA
CCTGGAGGTCGTCACGAGCACCTGGGTGCTGGTAGGCGGAGTCCTAGCAGCTCT
GGCCGCGTATTGCCTGACAACAGGCA
GCGTGGTCATTGTGGGCAGGATCATCTTGTCCGGAAAGCCGGCCATCATTCCCG
ACAGGGAAGTCCTTTACCGGGAGTTC
GATGAGATGGAAGAGTGCGCCTCACACCTCCCTTACATCGAACAGGGAATGCA
GCTCGCCGAACAATTCAAACAGAAGGC
AATCGGGTTGCTGCAAACAGCCACCAAGCAAGCGGAGGCTGCTGCTCCCGTGGT
GGAATCCAAGTGGCGGACCCTCGAAG
CCTCTGGGCGAAGCATATGTGGAATTTCATCAGCGGGATACAATATTTAGCAG
GCTTGTCCACTCTGCCTGGCAACCCC
GCGATAGCATCACTGATGGCATTCACAGCCTCTATCACCAGCCCGCTCACCACC
CAACATACCCTCCTGTTTAACATCCT
GGGGGGATGGGTGGCCGCCCAACTTGCTCCTCCCAGCGCTGCTTCTGCTTTCGT
AGGCGCCGGCATCGCTGGAGCGGCTG
TTGGCAGCATAGGCCTTGGGAAGGTGCTTGTGGATATTTTGGCAGGTTATGGAG
CAGGGGTGGCAGGCGCGCTCGTGGCC
TTTAAGGTCATGAGCGGCGAGATGCCCTCCACCGAGGACCTGGTTAACCTACTC
CCTGCTATCCTCTCCCCTGGCGCCCT
AGTCGTCGGGGTCGTGTGCGCAGCGATACTGCGTCGGCACGTGGGCCCAGGGG
AGGGGGGTGTGCAGTGGATGAACCGGC
TGATAGCGTTCGCTTCGCGGGGTAACCACGTCTCCCCCACGCACTATGTGCCTG
AGAGCGACGCTGCAGCACGTGTCACT
CAGATCCTCTCTAGTCTTACCATCACTCAGCTGCTGAAGAGGCTTCACCAGTGG
ATCAACGAGGACTGCTCCACGCCATG
CTCCGGCTCGTGGCTAAGAGATGTTTGGGATTGGATATGCACGGTGTTGACTGA
TTTCAAGACCTGGCTCCAGTCCAAGC
TCCTGCCGCGATTGCCGGGAGTCCCCTTCTTCTCATGTCAACGTGGGTACAAGG
GAGTCTGGCGGGGCGACGGCATCATG
CAAACCACCTGCCCATGTGGAGCACAGATCACCGGACATGTGAAAAACGGTTC
CATGAGGATCGTGGGGCCTAGGACCTG
TAGTAACACGTGGCATGGAACATTCCCCATTAACGCGTACACCACGGGCCCCTG
CACGCCCTCCCCGGCGCCAAATTATT
CTAGGGCGCTGTGGCGGGTGGCTGCTGAGGAGTACGTGGAGGTTACGCGGGTG
GGGGATTTCCACTACGTGACGGGCATG
ACCACTGACAACGTAAAGTGCCCGTGTCAGGTTCCGGCCCCCGAATTCTTCACG
GAGGTGGATGGAGTGCGGTTGCACAG
GTACGCTCCGGCGTGCAAACCTCTTCTACGGGAGGACGTCACGTTCCAGGTCGG
GCTCAACCAATACTTGGTCGGGTCGC
AGCTCCCATGCGAGCCCGAACCGGACGTAACAGTGCTTACTTCCATGCTCACCG
ATCCCTCCCACATTACAGCAGAGACG
GCTAAGCGTAGGCTGGCTAGAGGGTCTCCCCCCTCTTTAGCCAGCTCATCAGCT
AtCCAGTTGTCTGCGCCTTCTTTGAA
GGCGACATGCACTACCCACCATGACTCCCCGGACGCTGACCTCATCGAGGCCAA
CCTCTTGTGGCGGCAGGAGATGGGCG
GAAACATCACTCGCGTGGAGTCAGAGAATAAGGTAGTAATTCTGGACTCTTTCG
AACCGCTTCACGCGGAGGGGGATGAG
AGGGAGATATCCGTCGCGGCGGAGATCCTGCGAAAATCCAGGAAGTTCCCCTC
AGCGTTGCCCATATGGGCACGCCCGGA
CTACAATCCTCCACTGCTAGAGTCCTGGAAGGACCCGGACTACGTCCCTCCGGT
GGTACACGGATGCCCATTGCCACCTA
CCAAGGCTCCTCCAATACCACCTCCACGGAGAAAGAGGACGGTTGTCCTGACAG
AATCCAATGTGTCTTCTGCGTTGGCG
GAGCTCGCCACTAAGACCTTCGGTAGCTCCGGATCGTCGGCCGTTGATAGCGGC
ACGGCGACCGCCCTTCCTGACCTGGC
CTCCGACGACGGTGACAAAGGATCCGACGTTGAGTCGTACTCGTCCATGCCCCC
CCTTGAAGGGGAGCCGGGGGACCCCG
ATCTCAGCGACGGGTCTTGGTCTACCGTGAGTGAGGAGGCTAGTGAGGATGTCG
TCTGCTGCTCAATGTCCTATACGTGG
ACAGGCGCCCTGATCACGCCATGCGCTGCGGAGGAAAGTAAGCTGCCCATCAA
CCCGTTGAGCAACTCTTTGCTGCGTCA
CCACAACATGGTGTACGCCACAACATCCCGCAGCGCAAGCCTCCGGCAGAAGA
AGGTCACCTTTGACAGATTGCAAGTCC
TGGATGATCATTACCGGGACGTACTCAAGGAGATGAAGGCGAAGGCGTCCACA
GTTAAGGCTAAGCTTCTATCTATAGAG
GAGGCGTGCAAGCTGACGCCCCCACATTCGGCCAAATCCAAATTTGGCTATGGG
GCAAAGGACGTCCGGAACCTATCCAG
CAGGGCCGTTAACCACATCCGCTCCGTGTGGGAGGACTTGCTGGAAGACACTGA
AACACCAATTGACACCACCATCATGG
CAAAAAGTGAGGTTTTCTGCGTCCAACCAGAGAAGGGAGGCCGCAAGCCAGCT
CGCCTTATCGTATTCCCAGACCTGGGA
GTTCGTGTATGCGAGAAGATGGCCCTTTACGACGTGGTCTCCACCCTTCGTCAG
GCCGTGATGGGCTCCTCATACGGATT
TCAATAGTCCCCCAAGCAGCGGGTCGAGTTCCTGGTGAATACCTGGAAATCAAA
GAAATGCCCTATGGGCTTCTCATATG
ACACCCGCTGTTTTGACTCAACGGTCACTGAGAGTGACATTCGTGTTGAGGAGT
CAATTTACCAATGTTGTGACTTGGCC
CCCGAGGCCAGACAGGCCATAAGGTCGCTCACAGAGCGGCTTTACATCGGGGG
TCCCGTGACTAACTCAAAAGGGCAGAA
CTGCGGTTATCGCCGGTGCCGCGCAAGTGGCGTGCTGACGACTAGCTGCGGTAA
TACCCTCACATGTTACTTGAAGGCCA
CTGCAGCGTGTCGAGCTGCAAAGCTCCAGGAGTGCACGATGCTCGTGAACGGAG
ACGACCTTGTCGTTATGTGTGAAAGC
GCGGGAACCCAGGAGGATGCGGCGGCCCTACGAGCCTTCACGGAGGCTATGAC
TAGGTATTCCGCCCCCCCCGGGGATCC
GCCCCAACCAGAATACGACCTGGAGCTGATAACATCATGTTCCTCCAATGTGTC
AGTCGCGCACGATGCATCTGGCAAAA
GGGTATACTACCTCACCCGTGACCCCACCACCCCCCTTGCACGGGCTGCGTGGG
AGACAGCTAGACACACTCCAATCAAC
TCTTGGCTAGGCAATATCATCATGTATGCGCCCACCCTATGGGCAAGGATGATT
CTGATGACTCACTTTTTCTCCATCCT
TCTAGCTCAAGAGCAACTTGAAAAAGCCCTGGATTGTCAGATCTACGGGGCTTG
CTACTCCATTGAGCCACTTGACCTAC
CTCAGATCATTGAACGACTCCATGGTCTTAGCGCATTTACAGTCCACAGTTACTC
TCCAGGTGAGATCAATAGGGTGGCT
TCATGCCTCAGGAAACTTGGGGTACCACCCTTGCGAACCTGGAGACATCGGGCC
AGAAGTGTCCGCGCTAAGCTACTGTC
CCAGGGGGGGAGGGCCGCCACTTGTGGCAGATACCTGTTTAAGTGGGCAGTAA
GGACCAAGCTTAAACTCACTCCAATCC
CGGCCGCGTCCCAGCTGGACTTGTCTGGCTGGTTCGTCGCTGGTTACAGCGGGG
GAGACATATATCACAGCGTGTCTCGT
GCCCGACCCCGCTGGTTTCCGTTGTGCCTAGTCCTACTTTGTGTAGGGGTAGGCA
TTTACCTGCTCCCCAACCGATGAAC
GGGGAGCTAACCACTCCAGGCCTTAAGCCATTTCCTGTTTTTTTTTTTTTTTTTTT
TTTTTTTTTCTTTTTTTTTTTCTT
TCCTTTCCTTCTTTTTTTCCTTTCTTTTTCCCTTCTTTAATGGTGGCTCCATCTTAG
CCCTAGTCACGGCTAGCTGTGAA
AGGTCCGTGAGCCGCATGACTGCAGAGAGTGCTGATACTGGCCTCTCTGCAGAT
CAaGT
J4B/R1 (C): [SEQ ID NO: 10]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTCTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTGTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAAGTGCAGGACGAGGCAGCGCGGCTATCGTGGCTGGCCACGACGGGCGCGTTCC
TTGCGCAGCTGTGCTCGACGTTGTCAC
TGAAGCGGGAAGGGACTGGCTGCTATTGGGCGAAGTGCCGGGGCAGGATCTCC
TGTCATCTCACCTTGCTCCTGCCGAGA
AAGTATCCATCATGGCTGATGCAATGCGGCGGCTGCATACGCTTGATCCGGCTA
CCTGCCCATfCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATGTCGTCGTGACCCATGGCGATGCCT
GCTTGCCGAATATCATGGTGGAAAATGGCCGCTTTTGTGGATTCATCGAGTGTG
GCCGGCTGGGTGTGGCGGACCGCTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGCTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTGGACCAGGACCTCGTCG
GCTGGCAAGCGCCCCCCGGGGCGCGTT
CCTTGACACCATGCACCTGCGGCAGCTCGGACCTTTACTTGGTCACGAGGCATG
CCGATGTCATTCCGGTGCGCCGGCGG
GGCGACAGCAGGGGGAGCCTACTCTCCCCCAGGCCCGTCTCGTACTTGAAGGGC
TCTTCGGGCGGTCCACTGCTCTGCCC
CTCGGGGCACGCTGTGGGCATCTTTCGGGCTGCCGTGTGCACCCGAGGGGTTGC
GAAGGCGGTGGAGTTTGTACCCGTCG
AGTCTATGGAAACCACTATGCGGTCCCCGGTCTTCACGGACAACTCGTCCCCTC
CGGCCGTACCGCAGACATTCCAGGTG
GCCCATCTACACGCCCCTACTGGTAGCGGCAAGAGCACTAAGGTGCCGGCTGCG
TATGCAGCCCAAGGGTATAAGGTGCT
TGTCCTGAACCCGTCCGTCGCCGCCACCCTAGGTTTCGGGGCGTATATGTCTAA
GGCACATGGTATCGACCCTAACATCA
GAACCGGGGTAAGGACCATCACCACGGGTGCCCCCATCACGTACTCCACCTATG
GCAAGTTTCTTGCCGACGGTGGTTGC
TCTGGGGGCGCCTATGACATCATAATATGTGATGAGTGCCACTCAAGTGACTCG
ACCACTATCCTGGGCATCGGCACAGT
CCTGGACCAAGCGGAGACGGCTGGAGCGCGACTCGTCGTGCTCGCCACCGCTA
CGCCTCCGGGATCGGTCACCGTGCCAC
ATCCAAACATCGAGGAGGTGGCTCTGTCCAGCACTGGAGAAATCCCCTTTTATG
GCAAAGCCATCCCCATCGAGACCATC
AAGGGGGGGAGGCACCTCATTTTTCTGCCATTCCAAGAAGAAATGTGATGAGCTC
GCCGCGAAGCTGTCCGGCCTCGGACT
CAATGCTGTAGCATATTACCGGGGCCTTGATGTATCCGTCATACCAACTAGCGG
AGACGTCATTGTCGTAGCAACGGACG
CTCTAATGACGGGCTTTACCGGCGATTTCGACTCAGTGATCGACTGCAATACAT
GTGTCACCCAGACAGTCGACTTCAGC
CTGGACCCGACCTTCACCATTGAGACGACGACCGTGCCACAAGACGCGGTGTCA
CGCTCGCAGCGGCGAGGCAGGACTGG
TAGGGGCAGGATGGGCATTTACAGGTTTGTGACTCCAGGAGAACGGCCCTCGG
GCATGTTCGATTCCTCGGTTCTGTGCG
AGTGCTATGACGCGGGCTGTGCTTGGTACGAGCTCACGCCCGCCGAGACCTCAG
TTAGGTTGCGGGCTTACGTAAACACA
CCAGGGTTGCCCGTCTGCCAGGACCATCTGGAGTTCTGGGAGAGCGTCTTTACA
GGCCTCACCCACATAGACGCCCATTT
CTTGTCCCAGACTAAGCAGGCAGGAGACAACTTCCCCTACCTGGTAGCATACCA
GGCTACGGTGTGCGCCAGGGCTCAGG
CTCCACCTCCATCGTGGGACCAAATGTGGAAGTGTCTCATACGGCTAAAGCCTA
CGCTGCACGGGCCAACGCCCCTGCTG
TATAGGCTGGGAGCCGTTCAAAACGAGGTTACTACCACACACCCCATAACCAAA
TACATCATGGCATGCATGTCGGCTGA
CCTGGAGGTCGTCACGAGCACCTGGGTGCTGGTAGGCGGAGTCCTAGCAGCTCT
GGCCGCGTATTGCCTGACAACAGGCA
GCGTGGTCATTGTGGGCAGGATCATCTTGTCCGGAAAGCCGGCCATCATTCCCG
ACAGGGAAGTCCTTTACCGGGAGTTC
GATGAGATGGAAGAGTGCGCCTCACACCTCCCTTACATCGAACAGGGAATGCA
GCTCGCCGAACAATTCAAACAGAAGGC
AATCGGGTTGCTGCAAACAGCCACCAAGCAAGCGGAGGCTGCTGCTCCCGTGGT
GGAATCCAAGTGGCGGACCCTCGAAG
CCTTCTGGGCGAAGCATATGTGGAATTTCATCAGCGGGATACAATATTTAGCAG
GCTTGTCCACTCTGCCTGGCAACCCC
GCGATAGCATCACTGATGGCATTCACAGCCTCTATCACCAGCCCGCTCACCACC
CAACATACCCTCCTGTTTAACATCCT
GGGGGGATGGGTGGCCGCCCAACTTGCTCCTCCCAGCGCTGCTTCTGCTTTCGT
AGGCGCCGGCATCGCTGGAGCGGCTG
TTGGCAGCATAGGCCTTGGGAAGGTGCTTGTGGATATTTTGGCAGGTTATGGAG
CAGGGGTGGCAGGCGCGCTCGTGGCC
TTTAAGGTCATGAGCGGCGAGATGCCCTCCACCGAGGACCTGGTTAACCTACTC
CCTGCTATCCTCTCCCCTGGCGCCCT
AGTCGTCGGGGTCGTGTGCGCAGCGATACTGCGTCGGCACGTGGGCCCAGGGG
AGGGGGCTGTGCAGTGGATGAACCGGC
TGATAGCGTTCGCTTCGCGGGGTAACCACGTCTCCCCCACGCACTATGTGCCTG
AGAGCGACGCTGCAGCACGTGTCACT
CAGATCCTCTCTAGTCTTACCATCACTCAGCTGCTGAAGAGGCTTCACCAGTGG
ATCAACGAGGACTGCTCCACGCCATG
CTCCGGCTCGTGGCTAAGAGATGTTTGGGATTGGATATGCACGGTGTTGACTGA
TTTCAAGACCTGGCTCCAGTCCAAGC
TCCTGCCGCGATTGCCGGGAGTCCCCTTCTTCTCATGTCAACGTGGGTACAAGG
GAGTCTGGCGGGGCGACGGCATCATG
CAAACCACCTGCCCATGTGGAGCACAGATCACCGGACATGTGAAAAACGGTTC
CATGAGGATCGTGGGGCCTAGGACCTG
TAGTAACACGTGGCATGGAACATTCCCCATTAACGCGTACACCACGGCCCCTG
CACGCCCTCCCCGGCGCCAAATTATT
CTAGGGCGCTGTGGCGGGTGGCTGCTGAGGAGTACGTGGAGGTTACGCGGGTG
GGGGATTTCCACTACGTGACGGGCATG
ACCACTGACAACGTAAAGTGCCCGTGTCAGGTTCCGGCCCCCGAATTCTTCACG
GAGGTGGATGGAGTGCGGTTGCACAG
GTACGCTCCGGCGTGCAAACCTCTTCTACGGGAGGACGTCACGTTCCAGGTCGG
GCTCAACCAATACTTGGTCGGGTCGC
AGCTCCCATGCGAGCCCGAACCGGACGTAACAGTGCTTACTTCCATGCTCACCG
ATCCCTCCCACATTACAGCAGAGACG
GCTAAGCGTAGGCTGGCTAGAGGGTCTCCCCCCTCTTTAGCCAGCTCATCAGCT
AtCCAGTTGTCTGCGCCTTCTTTGAA
GGCGACATGCACTACCCACCATGACTCCCCGGACGCTGACGTCATCGAGGCCAA
CCTGTTGTGGCGGCAGGAGATGGGCG
GAAACATCACTCGCGTGGAGTCAGAGAATAAGGTAGTAATTCTGGACTCTTTCG
AACCGCTTCACGCGGAGGGGGATGAG
AGGGAGATATCCGTCGCGGCGGAGATCCTGCGAAAATCCAGGAAGTTCCCCTC
AGCGTTGCCCATATGGGCACGCCCGGA
CTACAATCCTCCACTGGTAGAGTCCTGGAAGGACCCGGACTACGTCCCTCCGGT
GGTACACGGATGCCCATTGCCACCTA
CCAAGGCTCCTCCAATACCACCTCCACGGAGAAAGAGGACGGTTGTCCTGACAG
AATCCAATGTGTCTTCTGCCTTGGCG
GAGCTCGCCACTAAGACCTTCGGTAGCTCCGGATCGTCGGCCGTTGATAGCGGC
ACGGCGACCGCCCTTCCTGACCTGGC
CTCCGACGACGGTGACAAAGGATCCGACGTTGAGTCGTACTCCTCCATGCCCCC
CCTTGAAGGGGAGCCGGGGGACCCCG
ATCTCAGCGACGGGTCTTGGTCTACCGTGAGTGAGGAGGCTAGTGAGGATGTCG
TCTGCTGCTCAATGTCCTATACGTGG
ACAGGCGCCCTGATCACGCCATGCGCTGCGGAGGAAAGTAAGCTGCCCATCAA
CCCGTTGAGCAACTCTTTGCTGCGTCA
CCACAACATGGTCTACGCCACAACATCCCGCAGCGCAAGCCTCCGGCAGAAGA
AGGTCACCTTTGACAGATTGCAAGTCC
TGGATGATCATTACCGGGACGTACTCAAGGAGATGAAGGCGAAGGCGTCCACA
GTTAAGGCTAAGCTTCTATCTATAGAG
GAGGCCTGCAAGCTGACGCCCCCACATTCGGCCAAATCCAAATTTGGCTATGGG
GCAAAGGACGTCCGGAACCTATCCAG
CAGGGCCGTTAACCACATCCGCTCCGTGTGGGAGGACTTGCTGGAAGACACTGA
AACACCAATTGACACCACCATCATGG
CAAAAAGTGAGGTTTTCTGCGTCCAACCAGAGAAGGGAGGCCGCAAGCCAGCT
CGCCTTATCGTATTCCCAGACCTGGGA
GTTCGTGTATGCGAGAAGATGGCCCTTTACGACGTGGTCTCCACCCTTCCTCAG
GCCGTGATGGGCTCCTCATACGGATT
TCAATACTCCCCCAAGCAGCGGGTCGAGTTCCTGGTGAATACCTGGAAATCAAA
GAAATGCCCTATGGGCTTCTCATATG
ACACCCGCTGTTTTGACTCAACGGTCACTGAGAGTGACATTCGTGTTGAGGAGT
CAATTTACCAATGTTGTGACTTGGCC
CCCGAGGCCAGACAGGCCATAAGGTCGCTCACAGAGCGGCTTTACATCGGGGG
TCCCCTGACTAACTCAAAAGGGCAGAA
CTGCGGTTATCGCCGGTGCCGCGCAAGTGGCGTGCTGACGACTAGCTGCGGTAA
TACCCTCACATGTTACTTGAAGGCCA
CTGCAGCCTGTCGAGCTGCAAAGCTCCAGGACTGCACGATGCTCGTGAACGGAG
ACGACCTTGTCGTTATCTGTGAAAGC
GCGGGAACCCAGGAGGATGCGGCGGCCCTACGAGCCTTCACGGAGGCTATGAC
TAGGTATTCCGCCCCCCCCGGGGATCC
GCCCCAACCAGAATACGACCTGGAGCTGATAACATCATGTTCCTCCAATGTGTC
AGTCGCGCACGATGCATCTGGCAAAA
GGGTATACTACCTCACCCGTGACCCCACCACCCCCCTTGCACGGGCTGCGTGGG
AGACAGCTAGACACACTCCAATCAAC
TCTTGGCTAGGCAATATCATCATGTATGCGCCCACCCTATGGGCAAGGATGATT
CTGATGACTCACTTTTTCTCCATCCT
TCTAGCTCAAGAGCAACTTGAAAAAGCCCTGGATTGTCAGATGTACGCGGGGTTG
CTACTCCATTGAGCCACTTGACCTAC
CTCAGATCATTGAACGACTCCATGGTCTTAGCGCATTTACACTCCACAGTTACTC
TCCAGGTGAGATCAATAGGGTGGCT
TCATGCCTCAGGAAACTTGGGGTACCACCCTTGCGAACCTGGAGACATCGGGCC
AGAAGTGTCCGCGCTAAGCTACTGTC
CCAGGGGGGGAGGGCCGCCACTTGTGGCAGATACCTCTTTAACTGGGCAGTAA
GGACCAAGCTTAAACTCACTCCAATCC
CGGCCGCGTCCCAGCTGGACTTGTCTGGCTGGTTCGTCGCTGGTTACAGCGGGG
GAGACATATATCACAGCCTGTCTCGT
GCCCGACCCCGCTGGTTTCCGTTGTGCCTACTCCTACTTTCTGTAGGGGTAGGCA
TTTACCTGCTCCCCAACCGATGAAC
GGGGAGCTAACCACTCCAGGCCTTAAGCCATTTCCTGTTTTTTTTTTTTTTTTTTT
TTTTTTTTTCTTTTTTTTTTTCTT
TCCTTTCCTTCTTTTTTTCCTTTCTTTTTCCCTTCTTTAATGGTGGCTCCATCTTAG
CCCTAGTCACGGCTAGCTGTGAA
AGGTCCGTGAGCCGCtTGACTGCAGAGAGTGCTGATACTGGCCTCTCTGCAGATC
AaGT
J4 Replicon: [SEQ ID NO: 11]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTCTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGCTATCGTGGCTGGCCACGACGGGCGTTCC
TTGCGCAGCTGTGCTCGACGTTGTCAC
TGAAGCGGGAAGGGACTGGCTGCTATTGGGCGAAGTGCCGGGGCAGGATCTCC
TGTCATCTCACCTTGCTCCTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGCACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCCT
GCTTGCCGAATATCATGGTGGAAAATGGCCGCTTTTCTGGATTCATCGACTGTG
GCCGGCTGGGTGTGGCGGACCGCTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTAAACAGACCACAACG
GTTTCCCTCTAGCGGGATCAATTCCGCCCCTCTCCGTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGGTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTAGACCTGGACCTCGTCGG
CTGGCAGGCGCCCCCCGGGGCGCGCT
CCATGACACCATGCAGCTGTGGCAGCTCGGACCTTTACTTGGTCACGAGACATG
CTGATGTCATTCCGGTGCGCCGGCGA
GGCGACAGCAGGGGAAGTCTACTCTCCCCCAGGCCCGTCTCCTACCTGAAAGGC
TCCTCGGGTGGTCCATTGCTTTGCCC
TTCGGGGCACGTCGTGGGCGTCTTCCGGGCTGCTGTGTGCACCCGGGGGGTCGC
GAAGGCGGTGGACTTCATACCCGTTG
AGTCTATGGAAACTACCATGCGGTCTCCGGTGTTCACAGACAACTCAACCCCCC
CGGCTGTACCGCAGACATTCCAAGTG
GCACATCTGCACGCTCCTACTGGCAGCGGCAAGAGCACCAAAGTGCCGGCTGC
GTATGCAGCCCAAGGGTACAAGGTGCT
CGTCCTGAACCCGTCCGTTGCCGCCACCTTAGGGTTTGGGGCGTATATGTCCAA
GGCACACGGTATCGACCCTAACATCA
GAACTGGGGTAAGGACCATTACCACGGGCGGCTCCATTACGTACTCCACCTATG
GCAAGTTCCTTGCCGACGGTGGCTGT
TGTGGGGGCGCCTATGACATCATAATATGTGATGAGTGCCACTCAAGTGACTCG
ACTACCATCTTGGGCATCGGCACAGT
CCTGGACCAAGCGGAGACGGCTGGAGCGCGGCTCGTCGTGCTCGCCACCGCTA
CACCTCCGGGATCGGTTACCGTGCCAC
ACCCCAATATCGAGGAAATAGGCCTGTCCAACAATGGAGAGATCCCCTTGTATG
GCAAAGCCATCCCCATTGAGGCCATC
AAGGGGGGGAGGCATCTCATTTTCTGCCATTCCAAGAAGAAATGTGACGAGCTC
GCCGCAAAGCTGACAGGCCTCGGACT
GAACGCTGTAGCATATTACCGGGGCCTTGATGTGTCCGTCATACCGCCTATCGG
AGACGTCGTTGTCGTGGCAACAGACG
CTCTAATGACGGGTTTCACCGGCGATTTTGACTCAGTGATCGACTGCAATACAT
GTGTCACCCAGACAGTCGACTTCAGC
TTGGATCCCACCTTCACCATTGAGACGACGACCGTGCCCCAAGACGCGGTGTCG
CGCTCGCAACGGCGAGGTAGAACTGG
CAGGGGTAGGAGTGGCATCTACAGGTTTGTGACTCCAGGAGAACGGCCCTCGG
GCATGTTCGATTCTTCGGTCCTGTGTG
AGTGCTATGACGCGGGCTGTGCTTGGTATGAGCTCACGCCCGCTGAGACCTCGG
TTAGGTTGCGGGCTTACCTAAATACA
CCAGGGTTGCCCGTCTGCCAGGACCATCTGGAGTTCTGGGAGAGCGTCTTCACA
GGCCTCACCCACATAGATGCCCACTT
CCTGTCCCAGACTAAACAGGCAGGAGACAACTTTCCTTACCTGGTGGCATATCA
AGCTACAGTGTGCGCCAGGGCTCAAG
CTCCACCTCCATCGTGGGACCAAATGTGGAAGTGTCTCATACGGCTGAAACCTA
CACTGCACGGGCCAACACCCCTGCTG
TATAGGCTAGGAGCCGTCCAAAATGAGGTCATCCTCACACACCCCATAACTAAA
TACATCATGGCATGCATGTCGGCTGA
CCTGGAGGTCGTCAGTAGCACCTGGGTGGTGGTAGGCGGAGTCCTTGCAGCTTT
GGCCGCATACTGCCTGACGACAGGCA
GTGTGGTCATTGTGGGCAGGATCATCTTGTCCGGGAAGCCAGCTGTCGTTCCCG
ACAGGGAAGTCCTCTACCAGGAGTTC
GATGAGATGGAAGAGTGTGCCTCACAACTTCCTTACATCGAGCAGGGAATGCA
GCTCGCCGAGCAATTCAAGCAAAAGGC
GCTCGGGTTGTTGCAAACGGCCACCAAGCAAGCGGAGGCTGCTGCTCCCGTGGT
GGAGTCCAAGTGGCGAGCCCTTGAGA
CCTTCTGGGCGAAGCACATGTGGAATTTCATCAGCGGAATACAGTACCTAGCAG
GCTTATCCACTCTGCCTGGAAACCCC
GCGATAGCATCATTGATGGCATTTACAGCTTCTATCACTAGCCCGCTCACCACCC
AAAACACCCTCCTGTTTAACATGTT
GGGGGGATGGGTGGCTGCCCAACTCGCTCCTCCCAGCGCTGCGTCAGCTTTCGT
GGGCGCCGGCATCGCCGGAGCGGCTG
TTGGCAGCATAGGCCTTGGGAAGGTGCTCGTGGACATCTTGGCGGGCTATGGGG
CAGGGGTAGCCGGCGCACTCGTGGCC
TTTAAGGTCATGAGCGGCGAGGTGCCCTCCACCGAGGACCTGGTCAACTTACTC
CCTGCCATCCTCTCTCCTGGTGCCCT
GGTCGTCGGGGTCGTGTGCGCAGCAATACTGCGTCGGCACGTGGGCCCGGGAG
AGGGGGCTGTGCAGTGGATGAACCGGC
TGATAGCGTTCGCTTCGCGGGGTAACCACGTCTCCCCTACGCACTATGTGCCTG
AGAGCGACGCTGCAGCACGTGTCACT
CAGATCCTCTCTAGCCTTACCATCACTCAACTGGTGAAGCGGCTCCACCAGTGG
ATTAATGAGGACTGCTCTACGCCATG
CTCCGGCTCGTGGCTAAGGGATGTTTGGGATTGGATATGCACGGTGTTGACTGA
CTTCAAGACCTGGCTCCAGTCCAAAC
TCCTGCCGCGGTTACCGGGAGTCCCTTTCCTGTCATGCCAACGCGGGTACAAGG
GAGTCTGGCGGGGGGACGGCATCATG
CAAACCACCTGCCCATGCGGAGCACAGATCGCCGGACATGTCAAAAACGGTTC
CATGAGGATCGTAGGGCCTAGAACCTG
CAGCAACACGTGGCACGGAACGTTCCCCATCAACGCATACACCACGGGACCTT
GCACACCCTCCCCGGCGCCCAACTATT
CCAGGGCGCTATGGCGGGTGGCTGCTGAGGAGTACGTGGAGGTTACGCGTGTG
GGGGATTTCCACTACGTGACGGGCATG
ACCACTGACAACGTAAAGTGCCCATGCCAGGTTCCGGCCCCCGAATTCTTCACG
GAGGTGGATGGAGTGCGGTTGCACAG
GTACGCTCCGGCGTGCAAACCTCTTCTACGGGAGGACGTCACGTTCCAGGTCGG
GCTCAACCAATACTTGGTCGGGTCGC
AGCTCCCATGCGAGCCCGAACCGGACGTAACAGTGCTTACTTCCATGCTCACCG
ATCCCTCCCACATTACAGCAGAGACG
GCTAAGCGTAGGCTGGCTAGAGGGTCTCCCCCCTCTTTAGCCAGCTCATCAGCT
AtCCAGTTGTCTGCGCCTTCTTTGAA
GGCGACATGCACTACCCACCATGACTCCCCGGACGCTGACCTCATCGAGGCCAA
CCTCTTGTGGCGGCAGGAGATGGGCG
GAAACATCACTCGCGTGGAGTCAGAGAATAAGGTAGTAATTCTGGACTCTTTCG
AACCGCTTCACGCGGAGGGGGATGAG
AGGGAGATATCCGTCGCGGCGGAGATCCTGCGAAAATCCAGGAAGTTCCCCTC
AGCGTTGCCCATATGGGCACGCCCGGA
CTACAATCCTCCACTGCTAGAGTCCTGGAAGGACCCGGACTACGTCCCTCCGGT
GGTACACGGATGCCCATTGCCACCTA
CCAAGGCTCCTCCAATACCACCTCCACGGAGAAAGAGGACGGTTGTCCTGACAG
AATCCAATGTGTCTTCTGCCTTGGCG
GAGCTCGCCACTAAGACCTTCGGTAGCTCCGGATCGTCGGCCGTTGATAGCGGC
ACGGCGACCGCCCTTCCTGACCTGGC
CTCCGACGACGGTGACAAAGGATCCGACGTTGAGTCGTACTCCTCCATGCCCCC
CCTTGAAGGGGAGCCGGGGGACCCCG
ATCTCAGCGACGGGTCTTGGTGTACCGTGAGTGAGGAGGCTAGTGAGGATGTCG
TCTGCTGCTCAATGTCCTATACGTGG
ACAGGCGCCCTGATCACGCCATGCGCTGCGGAGGAAAGTAAGCTGCCCATCAA
CCCGTTGAGCAACTTTTGCTGCGTCA
CCACAACATGGTCTACGCCACAACATCCCGCAGCGCAAGCCTCCGGCAGAAGA
AGGTCACCTTTGACAGATTGCAAGTCC
TGGATGATCATTACCGGGACGTACTCAAGGAGATGAAGGCGAAGGCGTCCACA
GTTAAGGCTAAGCTTCTATGTATAGAG
GAGGCCTGCAAGCTGACGCCCCCACATTCGGCCAAATCCAAATTTGGCTATGGG
GCAAAGGACGTCCGGAACCTATCCAG
CAGGGCCGTTAACCACATCCGCTCCGTGTGGGAGGACTTGCTGGAAGACACTGA
AACACCAATTGACACCACCATCATGG
CAAAAAGTGAGGTTTTCTGCGTCCAACCAGAGAAGGGAGGCCGCAAGCCAGCT
CGCCTTATCGTATCCCAGACCTGGGA
GTTCGTGTATGCGAGAAGATGGCCCTTTACGACGTGGTCTCCACCCTTCCTCAG
GCCGTGATGGGCTCCTCATACGGATT
TCAATACTCCCCCAAGCAGCGGGTCGAGTTCCTGGTGAATACCTGGAAATCAAA
GAAATGCCCTATGGGCTTCTCATATG
ACACCCGCTGTTTTGACTCAACGGTCACTGAGAGTGACATTCGTGTTGAGGAGT
CAATTTACCAATGTTGTGACTTGGCC
CCCGAGGCCAGACAGGCCATAAGGTCGCTCACAGAGCGGCTTTACATCGGGGG
TCCCCTGACTAACTCAAAAGGGCAGAA
CTGCGGTTATCGCCGGTGCCGCGCAAGTGGCGTGCTGACGACTAGCTGCGGTAA
TACCCTCACATGTTACTTGAAGGCCA
CTGCAGCCTGTCGAGCTGCAAAGCTCCAGGACTGCACGATGCTCGTGAACGGAG
ACGACCTTGTCGTTATGTGTGAAAGC
GCGGGAACCCAGGAGGATGCGGCGGCCCTACGAGCCTTCACGGAGGCTATGAC
TAGGTATTCCGCCCCCCCCGGGGATCC
GCCCCAACCAGAATACGACCTGGAGCTGATAACATCATGTTCCTCCAATGTGTC
AGTCGCGCACGATGCATCTGGCAAAA
GGGTATACTACCTCACCCGTGACCCCACCACCCCCCTTGCACGGGCTGCGTGGG
AGACAGCTAGACACACTCCAATCAAC
TCTTGGCTAGGCAATATCATCATGTATGCGCCCACCCTATGGGCAAGGATGATT
CTGATGACTCACTTTTTCTCATCCT
TCTAGCTCAAGAGCAACTTGAAAAAGCCCTGGATTGTCAGATCTACGGGGCTTG
CTACTCCATTGAGCCACTTGACCTAC
CTCAGATCATTGAACGACTCCATGGTCTTAGCGCATTTACACTCCACAGTTACTC
TCCAGGTGAGATCAATAGGGTGGCT
TCATGCCTCAGGAAACTTGGGGTACCACCCTTGCGAACCTGGAGACATCGGGCC
AGAAGTGTCCGCGCTAAGCTACTGTC
CCAGGGGGGGAGGGCCGCCACTTGTGGCAGATACCTCTTTAACTGGGCAGTAA
GGACCAAGCTTAAAGTCACTCCAATCC
CGGCCGCGTCCCAGCTGGACTTGTCTGGCTGGTTCGTCGCTGGTTACAGCGGGG
GAGACATATATCACAGCCTGTCTCGT
GCCCGACCCCGCTGGTTTCCGTTGTGCCTACTCCTACTTTCTGTAGGGGTAGGCA
TTTACCTGCTCCCCAACCGATGAAC
GGGGAGCTAACCACTCCAGGCCTTAAGCCATTTCCTGTTTTTTTTTTTTTTTTTTT
TCCTTTCCTTCTTTTTTTCCTTTCTTTTTCCCTTCTTTAATGGTGGCTCCATCTTAG
CCCTAGTCACGGCTAGCTGTGAA
AGGTCCGTGAGCCGCATGACTGCAGAGAGTGCTGATACTGGCCTCTCTGCAGAT
CAaGT
J4 replicon (c): [SEQ ID NO: 12]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTCTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGCTATCGTGGCTGGCCACGACGGGCGTTCC
TTGCGCAGCTGTGCTCGACGTTGTCAC
TGAAGCGGGAAGGGACTGGCTGCTATTGGGCGAAGTGCCGGGGCAGGATCTCC
TGTCATCTCACCTTGCTCCTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCCT
GCTTGCCGAATATCATGGTGGAAAATGGCCGCTTTTCTGGATTCATCGACTGTG
GCCGGCTGGGTGTGGCGGACCGGTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTGTGTTGAATGTCGTGAAGGAAGCA
GTTCCTCTGGAAGCTTCTTGAAGACAAACAACGTCTGTAGCGACCCTTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGCTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTAGACCTGGACCTCGTCGG
CTGGCAGGCGCCCCCCGGGGCGCGCT
CCATGACACCATGCAGCTGTGGCAGCTCGGACCTTTACTTGGTCACGAGACATG
CTGATGTCATTCCGGTGCGCCGGCGA
GGCGACAGCAGGGGAAGTCTACTCTCCCCCAGGCCCGTCTCCTACCTGAAAGGC
TCCTCGGGTGGTCCATTGCTTTGCCC
TTCGGGGCACGTCGTGGGCGTCTTCCGGGCTGGTGTGTGCACCCGGGGGGTCGC
GAAGGCGGTGGACTTCATACCCGTTG
AGTCTATGGAAACTACCATGCGGTCTCCGGTCTTCACAGACAACTCAACCCCCC
CGGCTGTACCGCAGACATTCCAAGTG
GCACATCTGCACGCTCCTACTGGCAGCGGCAAGAGCACCAAAGTGCCGGCTGC
GTATGCAGCCCAAGGGTACAAGGTGCT
CGTCCTGAACCCGTCCGTTGCCGCCACCTTAGGGTTTGGGGCGTATATGTCCAA
GGCACACGGTATCGACCCTAACATCA
GAACTGGGGTAAGGACCATTACCACGGGCGGCTCCATTACGTACTCCACCTATG
GCAAGTTCCTTGCCGACGGTGGCTGT
TCTGGGGGCGCCTATGACATCATAATATGTGATGAGTGCCACTCAACTGACTCG
ACTACCATCTTGGGCATCGGCACAGT
CCTGGACCAAGCGGAGACGGCTGGAGCGCGGCTCGTCGTGCTCGCCACCGCTA
CACCTCCGGGATCGGTTACCGTGCCAC
ACCCCAATATCGAGGAAATAGGCCTGTCCAACAATGGAGAGATCCCCTTCTATG
GCAAAGCCATCCCCATTGAGGCCATC
AAGGGGGGGAGGCATCTCATTTTCTGCCATTCCAAGAAGAAATGTGACGAGCTC
GCCGCAAAGCTGACAGGCCTCGGACT
GAACGCTGTAGCATATTACCGGGGCCTTGATGTGTCCGTCATACCGCCTATCGG
AGACGTCGTTGTCGTGGCAACAGACG
CTCTAATGACGGGTTTCACCGGCGATTTTGACTCAGTGATCGACTGCAATACAT
GTGTCACCCAGACAGTCGACTTCAGC
TTGGATCCCACCTTCACCATTGAGACGACGACCGTGCCCCAAGACGCGGTGTCG
CGCTCGCAACGGCGAGGTAGAACTGG
CAGGGGTAGGAGTGGCATCTACAGGTTTGTGACTCCAGGAGAACGGCCCTCGG
GCATGTTCGATTCTTCGGTCCTGTGTG
AGTGCTATGACGCGGGCTGTGCTTGGTATGAGCTCACGCCCGCTGAGACCTCGG
TTAGGTTGCGGGCTTACCTAAATACA
CCAGGGTTGCCCGTCTGCCAGGACCATCTGGAGTTCTGGGAGAGCGTCTTCACA
GGCCTCACCCACATAGATGCCCACTT
CCTGTCCCAGACTAAACAGGCAGGAGACAACTTTCCTTACCTGGTGGCATATCA
AGCTACAGTGTGCGCCAGGGCTCAAG
CTCCACCTCCATCGTGGGACCAAATGTGGAAGTGTCTCATACGGCTGAAACCTA
CACTGCACGGGCCAACACCCCTGCTG
TATAGGCTAGGAGCCGTCCAAAATGAGGTCATCCTCACACACCCCATAACTAAA
TACATCATGGCATGCATGTCGGCTGA
CCTGGAGGTCGTCACTAGCACCTGGGTGCTGGTAGGCGGAGTCCTTGCAGCTTT
GGCCGCATACTGCCTGACGACAGGCA
GTGTGGTCATTGTGGGCAGGATCATCTTGTCCGGGAAGCCAGCTGTCGTTCCCG
ACAGGGAAGTCCTCTACCAGGAGTTC
GATGAGATGGAAGAGTGTGCCTCACAACTTCCTTACATCGAGCAGGGAATGCA
GCTCGCCGAGCAATTCAAGCAAAAGGC
GCTCGGGTTGTTGCAAACGGCCACCAAGCAAGCGGAGGCTGCTGCTCCCGTGGT
GGAGTCCAAGTGGCGAGCCCTTGAGA
CCTTCTGGGCGAAGCACATGTGGAATTTCATCAGCGGAATACAGTACCTAGCAG
GCTTATCCACTCTGCCTGGAAACCCC
GCGATAGCATCATTGATGGCATTTACAGCTTCTATCACTAGCCCGCTCACCACCC
AAAACACCCTCCTGTTTAACATCTT
GGGGGGATGGGTGGCTGCCCAACTCGCTCCTCCCAGCGCTGCGTCAGCTTTCGT
GGGCGCCGGCATCGCCGGAGCGGCTG
TTGGCAGCATAGGCCTTGGGAAGGTGCTCGTGGACATCTTGGCGGGCTATGGGG
CAGGGGTAGCCGGCGCACTCGTGGCC
TTTAAGGTCATGAGCGGCGAGGTGCCCTCCACCGAGGACCTGGTCAACTTACTC
CCTGCCATCCTCTCTCCTGGTGCCCT
GGTCGTCGGGGTCGTGTGCGCAGCAATACTGCGTCGGCACGTGGGCCCGGGAG
AGGGGGCTGTGCAGTGGATGAACCGGC
TGATAGCGTTCGCTTCGCGGGGTAACCACGTCTCCCGTACGCACTATGTGCCTG
AGAGCGACGCTGCAGCACGTGTCACT
CAGATCGTCTGTAGCCTTACCATCACTCAACTGCTGAAGCGGCTCCACCAGTGG
ATTAATGAGGACTGCTCTACGCCATG
CTCCGGCTCGTGGCTAAGGGATGTTTGGGATTGGATATGCACGGTGTTGACTGA
CTTCAAGACCTGGCTCCAGTCCAAAC
TCCTGCCGCGGTTACCGGGAGTCCCTTTCGTGTCATGCCAACGCGGGTACAAGG
GAGTCTGGCGGGGGGACGGCATCATG
CAAACCACCTGCCCATGCGGAGCACAGATCGCCGGACATGTCAAAAACGGTTC
CATGAGGATCGTAGGGCCTAGAACCTG
CAGCAACACGTGGCACGGAACGTTCCCCATCAACGCATACACCACGGGACCTT
GCACACCCTCCCCGGCGCCCAACTATT
CCAGGGCGCTATGGCGGGTGGCTGCTGAGGAGTACGTGGAGGTTACGCGTGTG
GGGGATTTCCACTACGTGACGGGCATG
ACCACTGACAACGTAAAGTGCCCATGCCAGGTTCCGGCCCCCGAATTCTTCACG
GAGGTGGATGGAGTGCGGTTGCACAG
GTACGCTCCGGCGTGCAAACCTCTTCTACGGGAGGACGTCACGTTCCAGGTCGG
GCTCAACCAATACTTGGTCGGGTCGC
AGCTCCCATGCGAGCCCGAACCGGACGTAACAGTGCTTACTTCCATGCTCACCG
ATCCCTCCCACATTACAGCAGAGACG
GCTAAGCGTAGGCTGGCTAGAGGGTCTCCCCCCTCTTTAGCCAGCTCATCAGCT
AtCCAGTTGTCTGCGCCTTCTTTGAA
GGCGACATGCACTACCCACCATGACTCCCCGGACGCTGACCTCATCGAGGCCAA
CCTCTTGTGGCGGCAGGAGATGGGCG
GAAACATCACTCGCGTGGAGTCAGAGAATAAGGTAGTAATTCTGGACTCTTTCG
AACCGCTTCACGCGGAGGGGGATGAG
AGGGAGATATCCGTCGCGGCGGAGATCCTGCGAAAATCCAGGAAGTTCCCCTC
AGCGTTGCCCATATGGGCACGCCCGGA
CTACAATCCTCCACTGCTAGAGTCCTGGAAGGACCCGGACTACGTCCCTCCGGT
GGTACACGGATGCCCATTGCCACCTA
CCAAGGCTCCTCCAATACCACCTCCACGGAGAAAGAGGACGGTTGTCCTGACAG
AATCCAATGTGTCTTCTGCCTTGGCG
GAGCTCGCCACTAAGACCTTCGGTAGCTCCGGATCGTCGGCCGTTGATAGCGGC
ACGGCGACCGCCCTTCCTGACCTGGC
CTCCGACGACGGTGACAAAGGATCCGACGTTGAGTCGTACTCCTCCATGCCCCC
CCTTGAAGGGGAGCCGGGGGACCCCG
ATGTCAGCGACGGGTCTTGGTCTACCGTGAGTGAGGAGGCTAGTGAGGATGTCG
TCTGCTGCTCAATGTCGTATACGTGG
ACAGGCGCCCTGATCACGCCATGCGCTGCGGAGGAAAGTAAGCTGCCCATCAA
CCCGTTGAGCAACTCTTTGCTGCGTCA
CCACAACATGGTCTACGCCACAACATCCCGCAGCGCAAGCCTCCGGCAGAAGA
AGGTCACCTTTGACAGATTGCAAGTCC
TGGATGATCATTACCGGGACGTACTCAAGGAGATGAAGGCGAAGGCGTCCACA
GTTAAGGCTAAGCTTCTATCTATAGAG
GAGGCCTGCAAGCTGACGCCCCCACATTCGGCCAAATCCAAATTTGGCTATGGG
GCAAAGGACGTCCGGAACGTATCCAG
CAGGGCCGTTAACCACATCCGCTCCGTGTGGGAGGACTTGCTGGAAGACACTGA
AACACCAATTGACACCACCATCATGG
CAAAAAGTGAGGTTTTCTGCGTCCAACCAGAGAAGGGAGGCCGCAAGCCAGCT
CGCCTTATCGTATTCCCAGACCTGGGA
GTTCGTGTATGCGAGAAGATGGCCCTTTACGACGTGGTCTCCACCCTTCCTCAG
GCCGTGATGGGCTCCTCATACGGATT
TCAATACTCCCCCAAGCAGCGGGTCGAGTTCCTGGTGAATACCTGGAAATCAAA
GAAATGCCCTATGGGCTTCTCATATG
ACACCCGCTGTTTTGACTCAACGGTCACTGAGAGTGACATTCGTGTTGAGGAGT
CAATTTACCAATGTTGTGACTTGGCC
CCCGAGGCCAGACAGGCCATAAGGTCGCTCACAGAGCGGCTTTACATCGGGGG
TCCCCTGAGTAACTCAAAAGGGCAGAA
CTGCGGTTATCGCCGGTGCCGCGCAAGTGGCGTGCTGACGACTAGCTCGGGTAA
TACCCTCACATGTTACTTGAAGGCCA
CTGCAGCCTGTCGAGCTGCAAAGCTCCAGGACTGCACGATGCTCGTGAACGGAG
ACGACCTTGTCGTTATCTGTGAAAGC
GCGGGAACCCAGGAGGATGCGGCGGCCCTACGAGCCTTCACGGAGGCTATGAC
TAGGTATTCCGCCCCCCCCGGGGATCC
GCCCCAACCAGAATACGACCTGGAGCTGATAACATCATGTTCCTCCAATGTGTC
AGTCGCGCACGATGCATCTGGCAAAA
GGGTATACTACCTCACCCGTGACCCCACCACCCCCCTTGCACGGGCTGCGTGGG
AGACAGCTAGACACACTCCAATCAAC
TCTTGGCTAGGCAATATCATCATGTATGCGCCCACCCTATGGGCAAGGATGATT
CTGATGACTCACTTTTTCTCCATCCT
TCTAGCTCAAGAGCAACTTGAAAAAGCCCTGGATTGTCAGATCTACGGGGCTTG
CTACTCCATTGAGCCACTTGACCTAC
CTCAGATCATTGAACGACTCCATGGTCTTAGCGCATTTACACTCCACAGTTACTC
TCCAGGTGAGATCAATAGGGTGGCT
TCATGCCTCAGGAAACTTGGGGTACCACCCTTGCGAACCTGGAGACATCGGGCC
AGAAGTGTCCGCGCTAAGCTACTGTC
CCAGGGGGGGAGGGCCGCCACTTGTGGCAGATACCTCTTTAACTGGGCAGTAA
GGACCAAGCTTAAACTCACTCCAATCC
CGGCCGCGTCCCAGCTGGACTTGTCTGGCTGGTTCGTCGCTGGTTACAGCGGGG
GAGACATATATCACAGCCTGTCTCGT
GCCCGACCCCGCTGGTTTCCGTTGTGCCTACTCCTACTTTCTGTAGGGGTAGGCA
TTTACCTGCTCCCCAACCGATGAAC
GGGGAGCTAACCACTCCAGGCCTTAAGCCATTTCCTGTTTTTTTTTTTTTTTTTTT
TTTTTTTTTCTTTTTTTTTTTCTT
TCCTTTCCTTCTTTTTTTCCTTTCTTTTTCCCTTCTTTAATGGTGGCTCCATCTTAG
CCCTAGTCACGGCTAGCTGTGAA
AGGTCCGTGAGCCGCtTGACTGCAGAGAGTGCTGATAGTGGCCTCTCTGCAGATC
AaGT
BB7/J4NS5B: [SEQ ID NO: 13]
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCGTCCAGGACCCCCCCTCCCGGGA
GAGCCATAGTGGTCTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGCTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGGT
GTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGCTATCGTGGCTGGCCACGACGGGCGTTCC
TTGCGCAGCTGTGCTCGACGTTGTCAC
TGAAGCGGGAAGGGACTGGCTGCTATTGGGCGAAGTGCCGGGGCAGGATCTCC
TGTCATCTCACCTTGCTCCTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATGTCGTCGTGACCCATGGCGATGCCT
GCTTGCCGAATATCATGGTGGAAAATGGCCGCTTTTCTGGATTCATCGACTGTG
GCCGGCTGGGTGTGGCGGACCGCTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCCTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTGTGTAGCGACCCTTTGCAGG
CAGCGGAACCCCCCACCTGGCGACAG
GTGCCTGTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCGTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGCTGCATCATCACTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTGGACCAGGACCTCGTCG
GCTGGCAAGCGCCCCCCGGGGCGCGTT
CCTTGACACCATGCACCTGCGGCAGCTCGGACCTTTACTTGGTCACGAGGCATG
CCGATGTCATTCCGGTGCGCCGGCGG
GGCGACAGCAGGGGGAGCCTACTCTCCCCCAGGCCCGTCTCCTACTTGAAGGGC
TCTTCGGGCGGTCCACTGCTCTGCCC
CTCGGGGCACGCTGTGGGCATCTTTCGGGCTGCCGTGTGCACCCGAGGGGTTGC
GAAGGCGGTGGACTTTGTACCCGTCG
AGTCTATGGAAACCACTATGCGGTCCCCGGTCTTCACGGACAACTCGTCCCCTC
CGGCCGTACCGCAGACATTCCAGGTG
GCCCATCTACACGCCCCTACTGGTAGCGGCAAGAGCACTAAGGTGCCGGCTGCG
TATGCAGCCCAAGGGTATAAGGTGCT
TGTCCTGAACCCGTCCGTCGCCGCCACCCTAGGTTTCGGGGCGTATATGTCTAA
GGCACATGGTATCGACCCTAACATCA
GAACCGGGGTAAGGACCATCACCACGGGTGCCCCCATCACGTACTCCACCTATG
GCAAGTTTCTTGCCGACGGTGGTTGC
TCTGGGGGCGCCTATGACATCATAATATGTGATGAGTGCCACTCAACTGACTCG
ACCACTATCCTGGGCATCGGCACAGT
CCTGGACCAAGCGGAGACGGCTGGAGCGCGACTCGTCGTGCTCGCCACCGCTA
CGCCTCCGGGATCGGTCACCGTGCCAC
ATCCAAACATCGAGGAGGTGGCTCTGTCCAGCACTGGAGAAATCCCCTTTTATG
GCAAAGCCATCCCCATCGAGACCATC
AAGGGGGGGAGGCACCTCATTTTCTGCCATTCCAAGAAGAAATGTGATGAGCTC
GCCGCGAAGCTGTCCGGCCTCGGACT
CAATGCTGTAGCATATTACCGGGGCCTTGATGTATCCGTCATACCAACTAGCGG
AGACGTCATTGTCGTAGCAACGGACG
CTCTAATGACGGGCTTTACCGGCGATTTCGACTCAGTGATCGACTGCAATACAT
GTGTCACCCAGACAGTCGACTTCAGC
CTGGACCCGACCTTCACCATTGAGACGACGACCGTGCCACAAGACGCGGTGTCA
CGCTCGCAGCGGCGAGGCAGGACTGG
TAGGGGCAGGATGGGCATTTACAGGTTTGTGACTCCAGGAGAACGGCCCTCGG
GCATGTTCGATTCCTCGGTTCTGTGCG
AGTGCTATGACGCGGGCTGTGCTTGGTACGAGCTCACGCCCGCCGAGACCTCAG
TTAGGTTGCGGGCTTACCTAAACACA
CCAGGGTTGCCCGTCTGCCAGGACCATCTGGAGTTCTGGGAGAGCGTCTTTACA
GGCCTCACCCACATAGACGCCCATTT
CTTGTCCCAGACTAAGCAGGCAGGAGACAACTTCCCCTACCTGGTAGCATACCA
GGCTACGGTGTGCGCCAGGGCTCAGG
CTCCACCTCCATCGTGGGACCAAATGTGGAAGTGTCTCATACGGCTAAAGCCTA
CGCTGCACGGGCCAACGCCCCTGCTG
TATAGGCTGGGAGCCGTTCAAAACGAGGTTACTACCACACACCCCATAACCAAA
TACATCATGGCATGCATGTCGGCTGA
CCTGGAGGTCGTCACGAGCACCTGGGTGCTGGTAGGCGGAGTCCTAGCAGCTCT
GGCCGCGTATTGCCTGACAACAGGCA
GCGTGGTCATTGTGGGCAGGATCATCTTGTCCGGAAAGCCGGCCATCATTCCCG
ACAGGGAAGTCCTTTACCGGGAGTTC
GATGAGATGGAAGAGTGCGCCTCACACCTCCCTTACATCGAACAGGGAATGCA
GCTCGCCGAACAATTCAAACAGAAGGC
AATCGGGTTGCTGCAAACAGCCACCAAGCAAGCGGAGGCTGCTGCTCCCGTGGT
GGAATCCAAGTGGCGGACCCTCGAAG
CCTTCTGGGCGAAGCATATGTGGAATTTCATCAGCGGGATACAATATTTAGCAG
GCTTGTCCACTCTGCCTGGCAACCCC
GCGATAGCATCACTGATGGCATTCACAGCCTCTATCACCAGCCCGCTCACCACC
CAACATACCCTCCTGTTTAACATCCT
GGGGGGATGGGTGGCCGCCCAACTTGCTCCTCCCAGCGCTGCTTCTGCTTTCGT
AGGCGCCGGCATCGCTGGAGCGGCTG
TTGGCAGCATAGGCCTTGGGAAGGTGCTTGTGGATATTTTGGCAGGTTATGGAG
CAGGGGTGGCAGGCGCGCTCGTGGCC
TTTAAGGTCATGAGCGGCGAGATGCCCTCCACCGAGGACCTGGTTAACCTACTC
CCTGCTATCCTCTCCCCTGGCGCCCT
AGTCGTCGGGGTCGTGTGCGCAGCGATACTGCGTCGGCACGTGGGCCCAGGGG
AGGGGGCTGTGCAGTGGATGAACCGGC
TGATAGCGTTCGCTTCGCGGGGTAACCACGTCTCCCCCACGCACTATGTGCCTG
AGAGCGACGCTGCAGCACGTGTCACT
CAGATCCTCTCTAGTCTTACCATCACTCAGCTGCTGAAGAGGCTTCACCAGTGG
ATCAACGAGGACTGCTCCACGCCATG
CTCCGGCTCGTGGCTAAGAGATGTTTGGGATTGGATATGCACGGTGTTGACTGA
TTTCAAGACCTGGCTCCAGTCCAAGC
TCCTGCCGCGATTGCCGGGAGTCCCCTTCTTCTCATGTCAACGTGGGTACAAGG
GAGTCTGGCGGGGCGACGGCATCATG
CAAACCACCTGCCCATGTGGAGCACAGATCACCGGACATGTGAAAAACGGTTC
CATGAGGATCGTGGGGCCTAGGACCTG
TAGTAACACGTGGCATGGAACATTCCCCATTAACGCGTACACCACGGGCCCCTG
CACGCCCTCCCCGGCGCCAAATTATT
CTAGGGCGCTGTGGCGGGTGGCTGCTGAGGAGTACGTGGAGGTTACGCGGGTG
GGGGATTTCCACTACGTGACGGGCATG
ACCACTGACAACGTAAAGTGCCCGTGTCAGGTTCCGGCCCCCGAATTCTTCACA
GAAGTGGATGGGGTGCGGTTGCACAG
GTACGCTCCAGCGTGCAAACCCCTCCTACGGGAGGAGGTCACATTCCTGGTCGG
GCTCAATCAATACCTGGTTGGGTCAC
AGCTCCCATGCGAGCCCGAACCGGACGTAGCAGTGCTCACTTCCATGCTCACCG
ACCCCTCCCACATTACGGCGGAGACG
GCTAAGCGTAGGCTGGCCAGGGGATCTCCCCCCTCCTTGGCCAGCTCATCAGCT
ATCCAGCTGTCTGCGCCTTCCTTGAA
GGCAACATGCACTACCCGTCATGACTCCCCGGACGCTGACCTCATCGAGGCCAA
CCTCCTGTGGCGGCAGGAGATGGGCG
GGAACATCACCCGCGTGGAGTCAGAAAATAAGGTAGTAATTTTGGACTCTTTCG
AGCCGCTCCAAGCGGAGGAGGATGAG
AGGGAAGTATCCGTTCCGGCGGAGATCCTGCGGAGGTCCAGGAAATTCCCTCGA
GCGATGCCCATATGGGCACGCCCGGA
TTACAACCCTCCACTGTTAGAGTCCTGGAAGGACCCGGACTACGTCCCTCCAGT
GGTACACGGGTGTCCATTGCCGCCTG
CCAAGGCCCCTCCGATACCACCTCCACGGAGGAAGAGGACGGTTGTCCTGTCAG
AATCTACCGTGTCTTCTGCCTTGGCG
GAGCTCGCCACAAAGACCTTCGGCAGCTCCGAATCGTCGGCCGTCGACAGCGG
CACGGCAACGGCCTCTCCTGACCAGCC
CTCCGACGACGGCGACGCGGGATCCGACGTTGAGTCGTACTCCTCCATGCCCCC
CCTTGAGGGGGAGCCGGGGGATCCCG
ATCTCAGCGACGGGTCTTGGTCTACCGTAAGCGAGGAGGCTAGTGAGGACGTCG
TCTGCTGCTCGATGTCCTACACATGG
ACAGGCGCCCTGATCACGCCATGCGCTGCGGAGGAAAGTAAGCTGCCCATCAA
CCCGTTGAGCAACTCTTTGCTGCGTCA
CCACAACATGGTCTACGCCACAACATCCCGCAGCGCAAGCCTCCGGCAGAAGA
AGGTCACCTTTGACAGATTGCAAGTCC
TGGATGATCATTACCGGGACGTACTCAAGGAGATGAAGGCGAAGGCGTCCACA
GTTAAGGCTAAGCTTCTATCTATAGAG
GAGGCCTGCAAGCTGACGCCCCCACATTCGGCCAAATCCAAATTTGGCTATGGG
GCAAAGGACGTCCGGAACCTATCCAG
CAGGGCCGTTAACCACATCCGCTCCGTGTGGGAGGACTTGCTGGAAAGACACTGA
AACACCAATTGACACCACCATCATGG
CAAAAAGTGAGGTTTTCTGCGTCCAACCAGAGAAGGGAGGCCGCGCAAGCCAGCT
CGCCTTATCGTATTCCCAGACCTGGGA
GTTCGTGTATGCGAGAAGATGGCCCTTTACGACGTGGTCTCCACCCTTCCTCAG
GCCGTGATGGGCTCCTCATACGGATT
TCAATACTCCCCCAAGCAGCGGGTCGAGTTCCTGGTGAATACCTGGAAATCAAA
GAAATGCCCTATGGGCTTCTCATATG
ACACCCGCTGTTTTGACTCAACGGTCACTGAGAGTGACATTCGTGTTGAGGAGT
CAATTTACCAATGTTGTGACTTGGCC
CCCGAGGCCAGACAGGCCATAAGGTCGCTCACAGAGCGGCTTTACATCGGGGG
TCCCCTGACTAACTCAAAAGGGCAGAA
CTGCGGTTATCGCCGGTGCCGCGCAAGTGGCGTGCTGACGACTAGCTGCGGTAA
TACCCTCACATGTTACTTGAAGGCCA
CTGCAGCCTGTCGAGCTGCAAAGCTCCAGGACTGCACGATGCTCGTGAACGGAG
ACGACCTTGTCGTTATGTGTGAAAGC
GCGGGAACCCAGGAGGATGCGGCGGCCCTACGAGCCTTCACGGAGGCTATGAC
TAGGTATTCCGCCCCCCCCGGGGATCC
GCCCCAACCAGAATACGACCTGGAGCTGATAACATCATGTTCCTCCAATGTGTC
AGTCGCGCACGATGCATCTGGCAAAA
GGGTATACTACCTCACCCGTGACCCCACCACCCCCCTTGCACGGGCTGCGTGGG
AGACAGCTAGACACACTCCAATCAAC
TCTTGGCTAGGCAATATCATCATGTATGCGCCCACCCTATGGGCAAGGATGATT
CTGATGACTCACTTTTTCTCCATCCT
TCTAGCTCAAGAGCAACTTGAAAAAGCCCTGGATTGTCAGATCTACGGGGCTTG
CTACTCCATTGAGCCACTTGACCTAC
CTCAGATCATTGAACGACTCCATGGTCTTAGCGCATTTACACTCCACAGTTACTC
TCCAGGTGAGATCAATAGGGTGGCT
TCATGCCTCAGGAAACTTGGGGTACCACCCTTGCGAACCTGGAGACATCGGGCC
AGAAGTGTCCGCGCTAAGCTACTGTC
CCAGGGGGGGAGGGCCGCCACTTGTGGCAGATACCTCTTTAACTGGGCAGTAA
GGACCAAGCTTAAACTCACTCCAATCC
CGGCCGCGTCCCAGTGGACTTGTCTGGCTGGTTCGTCGCTGGTTACAGCGGGG
GAGACATATATCACAGCCTGTCTCGT
GCCCGACCCCGCTGGTTCCGTTGTGCCTACTCCTACTTTCTGTAGGGGTAGGCA
TTTACCTGCTCCCCAACCGATGAAC
GGGtAcgTAAACACTCCAGGCCAATAGGCCATCCTGTTTTTTTCCCTTTTTTTTTTT
CTTTTTTTTTTTTTTTTTTTTTT
TTTTTTTTTTTTCTCCTTTTTTTTTCCTCTTTTTTTCCTTTTCTTTCCTTTGGTGGCT
CCATCTTAGCCCTAGTCACGGC
TAGCTGTGAAAGGTCCGTGAGCCGCTTGAGTGCAGAGAGTGCTGATACTGGCCT
CTCTGCAGATCAAGT
pBB7-SN: [SEQ ID NO: 14]
BB7-SN:
GCCAGCCCCCGATTGGGGGCGACACTCCACCATAGATCACTCCCCTGTGAGGAA
CTACTGTCTTCACGCAGAAAGCGTCT
AGCCATGGCGTTAGTATGAGTGTCGTGCAGCCTCCAGGACCCCCCCTCCCGGGA
GAGCCATAGTGGTCTGCGGAACCGGT
GAGTACACCGGAATTGCCAGGACGACCGGGTCCTTTCTTGGATCAACCCGCTCA
ATGCCTGGAGATTTGGGCGTGCCCCC
GCGAGACTGCTAGCCGAGTAGTGTTGGGTCGCGAAAGGCCTTGTGGTACTGCCT
GATAGGGTGGTTGCGAGTGCCCCGGG
AGGTCTCGTAGACCGTGCACCATGAGCACGAATCCTAAACCTCAAAGAAAAAC
CAAAGGGCGCGCCATGATTGAACAAGA
TGGATTGCACGCAGGTTCTCCGGCCGCTTGGGTGGAGAGGCTATTCGGCTATGA
CTGGGCACAACAGACAATCGGCTGCT
CTGATGCCGCCGTGTTCCGGCTGTCAGCGCAGGGGCGCCCGGTTCTTTTTGTCAA
GACCGACCTGTCCGGTGCCCTGAAT
GAACTGCAGGACGAGGCAGCGCGGCTATCGTGGCTGGCCACGACGGGCGTTCC
TTTGCGCAGCTGTGCTCGACGTTGTCAC
TGAAGCGGGAAGGGACTGGCTGCTATTGGGCGAAGTGCCGGGGCAGGATCTCC
TGTCATCTCACCTTGCTCCTGCCGAGA
AAGTATCCATCATGGCTGATGCAATGCGGCGGCTGCATACGCTTGATCCGGCTA
CCTGCCCATTCGACCACCAAGCGAAA
CATCGCATCGAGCGAGCACGTACTCGGATGGAAGCCGGTCTTGTCGATCAGGAT
GATCTGGACGAAGAGCATCAGGGGCT
CGCGCCAGCCGAACTGTTCGCCAGGCTCAAGGCGCGCATGCCCGACGGCGAGG
ATCTCGTCGTGACCCATGGCGATGCCT
GCTTGCCGAATATCATGGTGGAAAATGGCCGCTTTTCTGGATTCATCGACTGTG
GCCGGCTGGGTGTGGCGGACCGCTAT
CAGGACATAGCGTTGGCTACCCGTGATATTGCTGAAGAGCTTGGCGGCGAATGG
GCTGACCGCTTCCTCGTGCTTTACGG
TATCGCCGCTCCCGATTCGCAGCGCATCGCCTTCTATCGCGTTCTTGACGAGTTC
TTCTGAGTTTAAACAGACCACAACG
GTTTCCCTCTAGCGGGATCAATTCCGCCCCTCTCCCTCCCCCCCCCCTAACGTTA
CTGGCCGAAGCCGCTTGGAATAAGG
CCGGTGTGCGTTTGTCTATATGTTATTTTCCACCATATTGCCGTCTTTTGGCAATG
TGAGGGCCCGGAAACCTGGCCCTG
TCTTCTTGACGAGCATTCCTAGGGGTCTTTCCCCTCTCGCCAAAGGAATGCAAG
GTCTGTTGAATGTCGTGAAGGAAGCA
GTTCCTCTGGAAGCTTCTTGAAGACAAACAACGTCTGTAGCGACCCTTTGCAGG
CAGCGGAACCCCCCACCTGGCGACAG
GTGCCTCTGCGGCCAAAAGCCACGTGTATAAGATACACCTGCAAAGGCGGCAC
AACCCCAGTGCCACGTTGTGAGTTGGA
TAGTTGTGGAAAGAGTCAAATGGCTCTCCTCAAGCGTATTCAACAAGGGGCTGA
AGGATGCCCAGAAGGTACCCCATTGT
ATGGGATCTGATCTGGGGCCTCGGTGCACATGCTTTACATGTGTTTAGTCGAGG
TTAAAAAACGTCTAGGCCCCCCGAAC
CACGGGGACGTGGTTTTCCTTTGAAAAACACGATAATACCATGGCGCCTATTAC
GGCCTACTCCCAACAGACGCGAGGCC
TACTTGGCTGCATCATCAGTAGCCTCACAGGCCGGGACAGGAACCAGGTCGAGG
GGGAGGTCCAAGTGGTCTCCACCGCA
ACACAATCTTTCCTGGCGACCTGCGTCAATGGCGTGTGTTGGACTGTCTATCATG
GTGCCGGCTCAAAGACCCTTGCCGG
CCCAAAGGGCCCAATCACCCAAATGTACACCAATGTGGACCAGGACCTCGTCG
GCTGGCAAGCGCCCCCCGGGGCGCGTT
CCTTGACACCATGCACCTGCGGCAGCTCGGACCTTTACTTGGTCACGAGGCATG
CCGATGTCATTCCGGTGCGCCGGCGG
GGCGACAGCAGGGGGAGCCTACTCTCCCCCAGGCCCGTCTCCTACTTGAAGGGC
TCTTCGGGCGGTCCACTGCTCTGCCC
CTCGGGGCACGCTGTGGGCATCTTTCGGGCTGCCGTGTGCACCCGAGGGGTTGC
GAAGGCGGTGGACTTTGTACCCGTCG
AGTCTATGGAAACCACTATGCGGTCCCCGGTCTTCACGGACAACTCGTCCCCTC
CGGCCGTACCGCAGACATTCCAGGTG
GCCCATCTACACGCCCCTACTGGTAGCGGCAAGAGCACTAAGGTGCCGGCTGCG
TATGCAGCCCAAGGGTATAAGGTGCT
TGTCCTGAACCCGTCCGTCGCCGCCACCCTAGGTTTCGGGGCGTATATGTCTAA
GGCACATGGTATCGACCCTAACATCA
GAACCGGGGTAAGGACCATCACCACGGGTGCCCCCATCACGTACTCCACCTATG
GCAAGTTTCTTGCCGACGGTGGTTGC
TCTGGGGGCGCCTATGACATCATAATATGTGATGAGTGCCACTCAACTGACTCG
ACCACTATCCTGGGCATCGGCACAGT
CCTGGACCAAGCGGAGACGGCTGGAGCGCGACTCGTCGTGCTCGCCACCGCTA
CGCCTCCGGGATCGGTCACCGTGCCAC
ATCCAAACATCGAGGAGGTGGCTCTGTCCAGCACTGGAGAAATCCCCTTTTATG
GCAAAGCCATCCCCATCGAGACCATC
AAGGGGGGGAGGCACCTCATTTTCTGCCATTCCAAGAAGAAATGTGATGAGCTC
GCCGCGAAGCTGTCCGGCCTCGGACT
CAATGCTGTAGCATATTACCGGGGCCTTGATGTATCCGTCATACCAACTAGCGG
AGACGTCATTGTCGTAGCAACGGACG
CTCTAATGACGGGCTTTACCGGCGATTTCGACTCAGTGATCGACTGCAATACAT
GTGTCACCCAGACAGTCGACTTCAGC
CTGGACCCGACCTTCACCATTGAGACGACGACCGTGCCACAAGACGCGGTGTCA
CGCTCGCAGCGGCGAGGCAGGACTGG
TAGGGGCAGGATGGGCATTTACAGGTTTGTGACTCCAGGAGAACGGCCCTCGG
GCATGTTCGATTCCTCGGTTCTGTGCG
AGTGCTATGACGCGGGCTGTGCTTGGTACGAGCTCACGCCCGCCGAGACCTCAG
TTAGGTTGCGGGCTTACCTAAACACA
CCAGGGTTGCCCGTCTGCCAGGACCATCTGGAGTTCTGGGAGAGCGTCTTTACA
GGCCTCACCCACATAGACGCCCATTT
CTTGTCCCAGACTAAGCAGGCAGGAGACAACTTCCCCTACCTGGTAGCATACCA
GGCTACGGTGTGCGCCAGGGCTCAGG
GTCCACCTCCATCGTGGGACCAAATGTGGAAGTGTCTCATACGGCTAAAGCCTA
CGCTGCACGGGCCAACGCCCCTGCTG
TATAGGCTGGGAGCCGTTCAAAACGAGGTTACTACCACACACCCCATAACCAAA
TACATCATGGCATGCATGTCGGCTGA
CCTGGAGGTCGTCACGAGCACCTGGGTGCTGGTAGGCGGAGTCCTAGCAGCTCT
GGCCGCGTATTGCCTGACAACAGGCA
GCGTGGTCATTGTGGGCAGGATCATCTTGTCCGGAAAGCCGGCCATCATTCCCG
ACAGGGAAGTCCTTTACCGGGAGTTC
GATGAGATGGAAGAGTGCGCCTCACACCTCCCTTACATCGAACAGGGAATGCA
GCTCGCCGAACAATTCAAACAGAAGGC
AATCGGGTTGCTGCAAACAGCCACCAAGCAAGCGGAGGCTGCTGCTCCCGTGGT
GGAATCCAAGTGGCGGACCCTCGAAG
CCTTCTGGGCGAAGCATATGTGGAATTTCATCAGCGGGATACAATATTTAGCAG
GCTTGTCCACTCTGCCTGGCAACCCC
GCGATAGCATCACTGATGGCATTCACAGCCTCTATCACCAGCCCGCTCACCACC
CAACATACCCTCCTGTTTAACATCCT
GGGGGGATGGGTGGCCGCCCAACTTGCTCCTCCCAGCGCTGCTTCTGCTTTCGT
AGGCGCCGGCATCGCTGGAGCGGCTG
TTGGCAGCATAGGCCTTGGGAAGGTGCTTGTGGATATTTTGGCAGGTTATGGAG
CAGGGGTGGCAGGCGCGCTCGTGGCC
TTTAAGGTCATGAGCGGCGAGATGCCCTCCACCGAGGACCTGGTTAACCTACTC
CCTGCTATCCTCTCCCCTGGCGCCCT
AGTCGTCGGGGTCGTGTGCGCAGCGATACTGCGTCGGCACGTGGGCCCAGGGG
AGGGGGCTGTGCAGTGGATGAACCGGC
TGATAGCGTTCGCTTCGCGGGGTAACCACGTCTCCCCCACGCACTATGTGCCTG
AGAGCGACGCTGCAGCACGTGTCACT
CAGATCCTCTCTAGTCTTACCATCACTCAGCTGCTGAAGAGGCTTCACCAGTGG
ATCAACGAGGACTGCTCCACGCCATG
CTCCGGCTCGTGGCTAAGAGATGTTTGGGATTGGATATGCACGGTGTTGACTGA
TTTCAAGACCTGGCTCCAGTCCAAGC
TCCTGCCGCGATTGCCGGGAGTCCCCTTCTTCTCATGTCAACGTGGGTACAAGG
GAGTCTGGCGGGGCGACGGCATCATG
CAAACCACCTGCCCATGTGGAGCACAGATCACCGGACATGTGAAAAACGGTTC
CATGAGGATCGTGGGGCCTAGGACCTG
TAGTAACACGTGGCATGGAACATTCCCCATTAACGCGTACACCACGGGCCCCTG
CACGCCCTCCCCGGCGCCAAATTATT
CTAGGGCGCTGTGGCGGGTGGCTGCTGAGGAGTACGTGGAGGTTACGCGGGTG
GGGGATTTCCACTACGTGACGGGCATG
ACCACTGACAACGTAAAGTGCCCGTGTCAGGTTCCGGCCCCCGAATTCTTCACA
GAAGTGGATGGGGTGCGGTTGCACAG
GTACGCTCCAGCGTGCAAACCCCTCCTACGGGAGGAGGTCACATTCCTGGTCGG
GCTCAATCAATACCTGGTTGGGTCAC
AGCTCCCATGCGAGCCCGAACCGGACGTAGCAGTGCTCACTTCCATGCTCACCG
ACCCCTCCCACATTACGGCGGAGACG
GCTAAGCGTAGGCTGGCCAGGGGATCTCCCCCGTCCTTGGCCAGCTCATCAGCT
ATCCAGCTGTCTGCGCCTTCCTTGAA
GGCAACATGCACTACCCGTCATGACTCCCCGGACGCTGACCTCATCGAGGCCAA
CCTCCTGTGGCGGCAGGAGATGGGCG
GGAACATCACCCGCGTGGAGTCAGAAAATAAGGTAGTAATTTTGGACTCTTTCG
AGCCGCTCCAAGCGGAGGAGGATGAG
AGGGAAGTATCCGTTCCGGCGGAGATCCTGCGGAGGTCCAGGAAATTCCCTCGA
GCGATGCCCATATGGGCACGCCCGGA
TTACAACCCTCCACTGTTAGAGTCCTGGAAGGACCCGGACTACGTCCCTCCAGT
GGTACACGGGTGTCCATTGCCGCCTG
CCAAGGCCCCTCCGATACCACCTCCACGGAGGAAGAGGACGGTTGTCCTGTCAG
AATCTACCGTGTCTTCTGCCTTGGCG
GAGCTCGCCACAAAGACCTTCGGCAGCTCCGAATCGTCGGCCGTCGACAGCGG
CACGGCAACGGCCTCTCCTGACCAGCC
CTCCGACGACGGCGACGCGGGATCCGACGTTGAGTCGTACTCCTCCATGCCCCC
CCTTGAGGGGGAGCCGGGGGATCCCG
ATCTCAGCGACGGGTCTTGGTCTACCGTAAGCGAGGAGGCTAGTGAGGACGTCG
TCTGCTGCTCGATGTCGTACACATGG
ACAGGCGCCCTGATCACGCCATGCGCTGCGGAGGAAACCAAGCTGCCCATCAA
TGCACTGAGCAACTCTTTGCTCCGTCA
CCACAACTTGGTCTATGGTACAACATCTCGCAGCGCAAGCCTGCGGCAGAAGAA
GGTCACCTTTGACAGACTGCAGGTCC
TGGACGACCACTACCGGGACGTGCTCAAGGAGATGAAGGCGAAGGCGTCCACA
GTTAAGGCTAAACTTCTATCCGTGGAG
GAAGCCTGTAAGCTGACGCCCCCACATTCGGCCAGATCTAAATTTGGCTATGGG
GCAAAGGACGTCCGGAACCTATCCAG
CAAGGCCGTTAACCACATCCGCTCCGTGTGGAAGGACTTGCTGGAAGACACTGA
GACACCAATTGACACCACCATCATGG
CAAAAAATGAGGTTTTCTGCGTCCAACCAGAGAAGGGGGGCCGCAAGCCAGCT
CGCCTTATCGTATTCCCAGATTTGGGG
GTTCGTGTGTGCGAGAAAATGGCCCTTTACGATGTGGTCTCCACCCTCCCTCAG
GCCGTGATGGGCTCTTCATACGGATT
CCAATACTCTCCTGGACAGCGGGTCGAGTTCCTGGTGAATGCCTGGAAAGCGAA
GAAATGCCCTATGGGCTTCGCATATG
ACACCCGCTGTTTTGACTCAACGGTCACTGAGAATGACATCCGTGTTGAGGAGT
CAATCTACCAATGTTGTGACTTGGCC
CCCGAAGCCAGACAGGCCATAAGGTCGCTCACAGAGCGGCTTTACATCGGGGG
CCCCCTGACTAATTCTAAAGGGCAGAA
CTGCGGCTATCGCCGGTGCCGCGCGAGCGGTGTACTGACGACCAGCTGCGGTAA
TACCCTCACATGTTACTTGAAGGCCG
CTGCGGCCTGTCGAGCTGCGAAGCTCCAGGACTGCACGATGCTCGTATGCGGAG
ACGACCTTGTCGTTATCTGTGAAAGC
GCGGGgACCCAAGAGGACGAGGCGAGCCTACGGGCCTTCACGGAGGCTATGAC
TAGATACTCTGCCCCCCCTGGGGACCC
GCCCAAACCAGAATACGACTTGGAGTTGATAACATCATGCTCCTCCAATGTGTC
AGTCGCGCACGATGCATCTGGCAAAA
GGGTGTACTATCTCACCCGTGACCCCACCACCCCCCTTGCGCGGGCTGCGTGGG
AGACAGCTAGACACACTCCAGTCAAT
TCCTGGCTAGGCAACATCATCATGTATGCGCCCACCTTGTGGGCAAGGATGATC
CTGATGACTCATTTCTTCTCCATCCT
TCTAGCTCAGGAACAACTTGAAAAAGCCCTAGATTGTCAGATCTACGGGGCCTG
TTACTCCATTGAGCCACTTGACCTAC
CTCAGATCATTCAACGACTCCATGGCCTTAGCGCATTTTCACTCCATAGTTACTC
TCCAGGTGAGATCAATAGGGTGGCT
TCATGCCTCAGGAAACTTGGGGTACCGCCCTTGCGAGTCTGGAGACATCGGGCC
AGAAGTGTCCGCGCTAGGCTACTGTC
CCAGGGGGGGAGGGCTGCCACTTGTGGCAAGTACCTCTTCAACTGGGCAGTAA
GGACCAAGCTCAAACTCACTCCAATCC
CGGCTGCGTCCCAGTTGGATTTATCCAGCTGGTTCGTTGCTGGTTACAGCGGGG
GAGACATATATCACAGCCTGTCTCGT
GCCCGACCCCGCTGGTTCATGTGGTGCCTACTCCTACTTTCTGTAGGGGTAGGCA
TCTATCTAGTCCCCAACCGATGAAC
GGGtACgTAAACACTCCAGGCCAATAGGCCATCCTGTTTTTTTCCCTTTTTTTTTT
TCTTTTTTTTTTTTTTTTTTTTTT
TTTTTTTTTTTTCTCCTTTTTTTTTCCTCTTTTTTTCCTTTTCTTTCCTTTGGTGGCT
CCATCTTAGCCCTAGTCACGGC
TAGCTGTGAAAGGTCCGTGAGCCGCTTGACTGCAGAGAGTGCTGATACTGGCCT
CTGTGCAGATCAAGT

(C) Primers

DNA primer sequences used in making the chimeric replicon constructs. Primers of the invention were derived from plasmids also described in the disclosure.

| | |
|---|---|
| BG1000: | |
| CAT CCA GAT GTA CAC CAA TGT GGA C | [SEQ ID NO: 31] |
| BG1001: | |
| CAT CGC CCG AAT TCT TCA CAG AAT TG | [SEQ ID NO: 32] |
| BG1002: | |
| CAA TTC TGT GAA GAA TTC GGG CGA TG | [SEQ ID NO: 33] |
| BG1003: | |
| GTA ACA CCA ATT GAC ACT ACC ATC | [SEQ ID NO: 34] |
| BG1004: | |
| GAT GGT AGT GTC TAT TGG TGT TAC | [SEQ ID NO: 35] |
| RB8000: | |
| GCA CTA GTA CTT GAT CTG CAG AGA | [SEQ ID NO: 36] |
| GGC CAG TAT CAG CAC TCT CTG CAG | |
| TCA AGC GG | |
| J4-9841T: | |
| CTT TAG CCA GCT CAT CAG CTA TCC | [SEQ ID NO: 37] |
| AGT TGT CTG CGC CTT C | |
| J49841TR: | |
| GAA GGC GCA GAC AAC TGG ATA GCT | [SEQ ID NO: 38] |
| GAT GAG CTG GCT AAA C | |
| 1aS9484I R: | |
| GAG ATG GAG CGG ACA GCT GGA TAG | [SEQ ID NO: 39] |
| CCG AGG AGC TGG CCA TAG AAG | |
| 1aS9484I: | |
| CTT CTA TGG CCA GCT CCT CGG CTA | [SEQ ID NO: 40] |
| TCC AGC TGT CCG CTC CAT CTC | |
| rb6000: | |
| CGT CTG CTG CTC GAT GTC CTA C | [SEQ ID NO: 41] |
| RB7801 | |
| 3′CTC CCC CAA CCG ATG AAC GGG TAC | [SEQ ID NQ: 42] |
| GTA AAC ACT CCA GGC CAA TAG | |
| 1A 10501 | |
| CCT GGA CAG GCG CAC TGA TCA CC | [SEQ ID NO: 43] |
| J4-10861 | |
| GAG GAC TTG CTG GAA GAC ACT G | [SEQ ID NO: 44] |
| BB7980 | |
| CAG GAG TAC TTG ATC TGC AGA GAG GC | [SEQ ID NQ: 45] |
| BG1005: | |
| GCA CTA GTA CTT GAT CTG CAG AGA | [SEQ ID NO: 46] |
| GGC | |
| RB7801 | |
| 5′CTA TTG GCC TGG AGT GTT TAC GTA | [SEQ ID NO: 47] |
| CCC GTT CAT CGG TTG GGG GAG | |
| Ralf Neo | |
| 5′TCA AGA CCG ACC TGT CCG GTG CCC | [SEQ ID NO: 48] |
| Ralf Neo | |
| 3′CTT GAG CCT GGC GAA CAG TTC GGC | [SEQ ID NO: 49 |
| GAPDH for | |
| ACC ACA GTC CAT GCC ATC AC | [SEQ ID NO: 50] |
| GAPDH rev | |
| TCC ACC ACC CTG TTG CTG TA | [SEQ ID NO: 51] |
| Neo^{R} fwd | |
| CCG GCT ACC TGC CCA TTC | [SEQ ID NO: 52] |
| Neo^{R} rev | |
| CCA GAT CAT CCG ATC GAC AAG | [SEQ ID NO: 53] |
| 5′FAM- | |
| ACA TCG CAT CGA GCG AGC ACG TAC- | [SEQ ID NO: 54] |
| TAMRA 3′ | |

The following is a list of documents related to the above disclosure and particularly to the experimental procedures and discussions.
Lohmann V., Korner, F. Koch, J., Herian U. Theilmann, L., and Bartenschlager, R. Replication of subgenomic hepatitis C virus RNAs in a hepatoma cell line. Science 285:110, 1999
Blight, K. J. Kolykhalov, A. A and Rice C. M. Efficient initiation of HCV RNA replication in cell culture. Science 290:1972-4, 2000
Guo J. Bichko, V. and Seeger C 2001 Effect of alpha interferon on the hepatitis C virus replicon. J Virol. Septenber 2001; 75(18):8516-23.
Yanagi, M, St claire, M., Shapiro, M, Emerson, S. U. Purcell, R. H. and Bukh, J. 1998, Transcripts of a chimeric cDNA clone of hepatitis C virus genotype 1b are infectious in vivo. Virology, 244:161-72.
Yanagi, M, Purcell, R. H., M, Emerson, S. U. and Bukh, J. 1997 Transcripts from a single full-length cDNA clone of hepatitis C virus are infectious when directly transfected into the liver of a chimpanzee. Proc Natl Acad Sci USA. 94:8738-43.
Yanagi, M, St claire, M., Emerson, S. U., Purcell, R. H., and Bukh, J. 1999. In vivo analysis of the 3′ untranslated region of the hepatitis C virus after in vivo mutagenesis of an infectious cDNA clone. PNAS 96:2291-2295
Simmonds, P. et al. 1994 A proposed system for the nomenclature of hepatitis C viral genotypes. Hepatology 19:1321-1324.
Lindsay, K. L. 1997. Therapy of hepatitis C: overview. Hepatology. 1997 71S-77S.
Choo, Q. L. Kuo, G., Weiner, A. J., Overby, L. R. Bradley, D. W. and Houghton, M. 1989 Isolation of a cDNA clone derived from a blood-borne non-A, non-B viral hepatitis genome. Science, 244:359-362.
Miller, R. H. and Purcell, R. H, 1990 Hepatitis C virus shares amino acid sequence similarity with pestiviruses and flaviviruses as well as members of two plant virus supergroups. Proc Natl Acad Sci USA. 87:2057-61.
Kuo, G. et al., 1989 An assay for circulating antibodies to a major etiologic virus of human non-A, non-B hepatitis. Science. Apr. 26, 1989; 244(4902):362-4.
Wang, C. and Siddiqui, A. 1995. Structure and function of the hepatitis C virus internal ribosome entry site. Curr Top Microbiol Immunol. 1995; 203:99-115.
Kolykhalov A. A. Feinstone, S. M. Rice C. M. 1996; Identification of a highly conserved sequence element at the 3′ terminus of hepatitis C virus genome RNA. J Virol. 1996 70:3363-71.
Tanaka, T. Kato, N., Cho, M. J., and Shimotohno, K. 1995; A. novel sequence found at the 3′ terminus of hepatitis C virus genome. Biochem. Biophys. Res. Commun. 215;744-749.
Tanaka, T., Kato, N., Cho, M. J., Sugiyama, K., and Shimotohno, K. 1996; Structure of the 3′ terminus of the hepatitis C virus genome. J Virol. 70:3307-12.
Yamada, N., K. Tanihara, A. Takada, T. Yorihuzi, M. Tsutsumi, H. Shimomura, T. Tsuji, T. Date 1996. Genetic Organization and Diversity of the 3′ Noncoding Region of the Hepatitis C Virus Genome, Virology, 223: 255-261.

1. A sub-genomic viral replicon comprising:
(a) a nucleic acid construct encoding chimeric HCV nonstructural protein, and
(b) an NS5B polymerase gene.

2. A replicon of claim 1 wherein the NS5B polymerase gene is from an HCV strain and linked in cis to a 3′UTR from said strain.

3. A replicon of claim 1 wherein the chimeric nonstructural proteins comprise a protein selected from the group consisting of NS3, NS4A, NS4B, NS5A, and NS5B.

4. A replicon of claim 1 comprising an NS3 nucleotide sequence that encodes the first 75 contiguous N-terminal amino acids of the NS3 of genotype 1b, of a BB7 strain.

5. A replicon of claim 1 wherein the NS3 N-terminal nucleotide sequence comprises
[SEQ ID NO: 1]
ATGGCGCCTATTACGGCCTACTCCCAACAGACGCGAGGCCTACTTGGC
TGCATCATCACTAGCCTCACAGGCCGGGACAGGAACCAGGTCGAGGG
GGAGGTCCAAGTGGTCTCCACCGCAACACAATCTTTCCTGGCGACCTG
CGTCAATGGCGTGTGTTGGACTGTCTATCATGGTGCCGGCTCAAAGAC
CCTTGCCGGCCCAAAGGGCCCAATCACCCAAATG.

6. A replicon of claim 4 wherein said NS3 N-terminal nucleotide sequence replaces the N-terminal first 225 nucleotides of an NS3 from any of six major HCV genotypes selected from the group consisting of HCV genotype 1, 2, 3, 4, 5 and 6.

7. A replicon of claim 6 wherein the NS3 is from HCV genotype 1a.

8. A replicon of claim 7 wherein the HCV genotype 1a is from an H77 strain.

9. A sub-genomic viral replicon comprising:
(a) a nucleic acid construct encoding chimeric HCV nonstructural proteins, and
(b) at least the C-terminal end of a strain specific NS5B polymerase gene linked in cis to a 3′LTR sequence from said strain.

10. A replicon of claim 9 wherein the chimeric nonstructural proteins comprise a protein selected from the group consisting of NS3, NS4A, NS4B, NS5A, and NS5B.

11. A replicon of claim 9 comprising an NS3 nucleotide sequence that encodes about the first 75 contiguous N-terminal amino acids of the NS3 of genotype 1b, of a BB7 strain.

12. A replicon of claim 9 wherein the NS3 N-terminal nucleotide sequence comprises
[SEQ ID No: 1]
ATGGCGCCTATTACGGCCTACTCCCAACAGACGCGAGGCCTACTTGGC
TGCATCATCACTAGCCTCACAGGCCGGGACAGGAACCAGGTCGAGGG
GGAGGTCCAAGTGGTCTCCACCGCAACACAATCTTTTCCTGGCGACCTG
CGTCAATGGCGTGTGTTGGACTGTCTATCATGGTGCCGGCTCAAAGAC
CCTTGCCGGCCCAAAGGGCCCAATCACCCAAATG.

13. A replicon of claim 11 wherein said NS3 N-terminal nucleotide sequence replaces the N-terminal first 225 nucleotides of an NS3 from any of six major HCV genotypes selected from the group consisting of HCV genotype 1, 2, 3, 4, 5, and 6.

14. A replicon of claim 13 wherein the NS3 is from HCV genotype 1b.

15. A replicon of claim 14 wherein the NS3, genotype 1b, is from a J4 strain.

16. A sub-genomic viral replicon comprising SEQ ID NO:2, SEQ ID NO:6, SEQ ID NO:7, or SEQ ID NO:8.

17. A method of generating a cell comprising a replicating chimeric sub-genomic viral replicon, said method comprising introducing said chimeric replicon into a cell.

18. A cell comprising a replicating chimeric sub-genomic viral replicon.

19. The cell of claim 18 wherein the HCV sub-genomic replicon comprises all of the non-structural HCV genes and none of the structural HCV genes.

20. A method of screening for compounds that modulate viral replication comprising the steps of:
a) administering a test compound to a cell of claim 18, and
b) determining whether said test compound modulates the replication of said chimeric replicon.

21. A method of screening for compounds that inhibit viral replication comprising the steps of
a) administering a test compound to a cell of claim 18, and
b) determining whether said test compound inhibits the replication of said chimeric sub-genomic viral replicon.


SEQUENCE LISTING (54 sequences)

SEQ ID NO: 1 - DNA, 225 bp, Artificial Sequence
  The nucleotide sequence encodes the first 75 contiguous N-terminal amino acids of HCV type 1b, strain BB7
atggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatg

SEQ ID NO: 2 - DNA, 7989 bp, Artificial Sequence
  Thepolynucleotide sequence encodes sequences from HCV H77 (BB7-F1)Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctatccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggacctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 3 - DNA, 7992 bp, Artificial Sequence
  Thepolynucleotide sequence encodes sequences from HCV H77(BB7-F1/F2) Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaattcttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctatccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggacctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 4 - DNA, 7980 bp, Artificial Sequence
  Thepolynucleotide sequence encodes sequences from HCV H77(BB7-F3)Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctatccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcaagt

SEQ ID NO: 5 - DNA, 7980 bp, Artificial Sequence
  Thepolynucleotide sequence encodes sequences from HCV H77 (BB7-F3(C)) Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctatccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcttgactgcagagagtgctgatactggcctctctgcagatcaagt

SEQ ID NO: 6 - DNA, 7989 bp, Artificial Sequence
  Thepolynucleotide sequence encodes sequences from HCV H77(BB7/H77NS5B)Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctatccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactcctccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggtttacgtaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 7 - DNA, 7983 bp, Artificial Sequence
  Thepolynucleotide sequence encodes sequences from HCV 1a Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgccccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtgggagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgtggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaattcttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctatccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaattgacactaccatcatggccaagaacgaggttttctgcgttcagcctgagaaggggggtcgtaagccagctcgtctcatcgtgttccccgacctgggcgtgcgcgtgtgcgagaagatggccctgtacgacgtgattagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggagacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactagatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcttgactgcagagagtgctgatactggcctctctgcagatcaagt

SEQ ID NO: 8 - DNA, 7989 bp, Artificial Sequence
  Thepolynucleotide sequence encodes sequences from HCV J4(J4 M/S)Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtagacctggacctcgtcggctggcaggcgccccccggggcgcgctccatgacaccatgcagctgtggcagctcggacctttacttggtcacgagacatgctgatgtcattccggtgcgccggcgaggcgacagcaggggaagtctactctcccccaggcccgtctcctacctgaaaggctcctcgggtggtccattgctttgcccttcggggcacgtcgtgggcgtcttccgggctgctgtgtgcacccggggggtcgcgaaggcggtggacttcatacccgttgagtctatggaaactaccatgcggtctccggtcttcacagacaactcaacccccccggctgtaccgcagacattccaagtggcacatctgcacgctcctactggcagcggcaagagcaccaaagtgccggctgcgtatgcagcccaagggtacaaggtgctcgtcctgaacccgtccgttgccgccaccttagggtttggggcgtatatgtccaaggcacacggtatcgaccctaacatcagaactggggtaaggaccattaccacgggcggctccattacgtactccacctatggcaagttccttgccgacggtggctgttctgggggcgcctatgacatcataatatgtgatgagtgccactcaactgactcgactaccatcttgggcatcggcacagtcctggaccaagcggagacggctggagcgcggctcgtcgtgctcgccaccgctacacctccgggatcggttaccgtgccacaccccaatatcgaggaaataggcctgtccaacaatggagagatccccttctatggcaaagccatccccattgaggccatcaagggggggaggcatctcattttctgccattccaagaagaaatgtgacgagctcgccgcaaagctgacaggcctcggactgaacgctgtagcatattaccggggccttgatgtgtccgtcataccgcctatcggagacgtcgttgtcgtggcaacagacgctctaatgacgggtttcaccggcgattttgactcagtgatcgactgcaatacatgtgtcacccagacagtcgacttcagcttggatcccaccttcaccattgagacgacgaccgtgccccaagacgcggtgtcgcgctcgcaacggcgaggtagaactggcaggggtaggagtggcatctacaggtttgtgactccaggagaacggccctcgggcatgttcgattcttcggtcctgtgtgagtgctatgacgcgggctgtgcttggtatgagctcacgcccgctgagacctcggttaggttgcgggcttacctaaatacaccagggttgcccgtctgccaggaccatctggagttctgggagagcgtcttcacaggcctcacccacatagatgcccacttcctgtcccagactaaacaggcaggagacaactttccttacctggtggcatatcaagctacagtgtgcgccagggctcaagctccacctccatcgtgggaccaaatgtggaagtgtctcatacggctgaaacctacactgcacgggccaacacccctgctgtataggctaggagccgtccaaaatgaggtcatcctcacacaccccataactaaatacatcatggcatgcatgtcggctgacctggaggtcgtcactagcacctgggtgctggtaggcggagtccttgcagctttggccgcatactgcctgacgacaggcagtgtggtcattgtgggcaggatcatcttgtccgggaagccagctgtcgttcccgacagggaagtcctctaccaggagttcgatgagatggaagagtgtgcctcacaacttccttacatcgagcagggaatgcagctcgccgagcaattcaagcaaaaggcgctcgggttgttgcaaacggccaccaagcaagcggaggctgctgctcccgtggtggagtccaagtggcgagcccttgagaccttctgggcgaagcacatgtggaatttcatcagcggaatacagtacctagcaggcttatccactctgcctggaaaccccgcgatagcatcattgatggcatttacagcttctatcactagcccgctcaccacccaaaacaccctcctgtttaacatcttggggggatgggtggctgcccaactcgctcctcccagcgctgcgtcagctttcgtgggcgccggcatcgccggagcggctgttggcagcataggccttgggaaggtgctcgtggacatcttggcgggctatggggcaggggtagccggcgcactcgtggcctttaaggtcatgagcggcgaggtgccctccaccgaggacctggtcaacttactccctgccatcctctctcctggtgccctggtcgtcggggtcgtgtgcgcagcaatactgcgtcggcacgtgggcccgggagagggggctgtgcagtggatgaaccggctgatagcgttcgcttcgcggggtaaccacgtctcccctacgcactatgtgcctgagagcgacgctgcagcacgtgtcactcagatcctctctagccttaccatcactcaactgctgaagcggctccaccagtggattaatgaggactgctctacgccatgctccggctcgtggctaagggatgtttgggattggatatgcacggtgttgactgacttcaagacctggctccagtccaaactcctgccgcggttaccgggagtccctttcctgtcatgccaacgcgggtacaagggagtctggcggggggacggcatcatgcaaaccacctgcccatgcggagcacagatcgccggacatgtcaaaaacggttccatgaggatcgtagggcctagaacctgcagcaacacgtggcacggaacgttccccatcaacgcatacaccacgggaccttgcacaccctccccggcgcccaactattccagggcgctatggcgggtggctgctgaggagtacgtggaggttacgcgtgtgggggatttccactacgtgacgggcatgaccactgacaacgtaaagtgcccatgccaggttccggcccccgaattcttcacggaggtggatggagtgcggttgcacaggtacgctccggcgtgcaaacctcttctacgggaggacgtcacgttccaggtcgggctcaaccaatacttggtcgggtcgcagctcccatgcgagcccgaaccggacgtaacagtgcttacttccatgctcaccgatccctcccacattacagcagagacggctaagcgtaggctggctagagggtctcccccctctttagccagctcatcagctatccagttgtctgcgccttctttgaaggcgacatgcactacccaccatgactccccggacgctgacctcatcgaggccaacctcttgtggcggcaggagatgggcggaaacatcactcgcgtggagtcagagaataaggtagtaattctggactctttcgaaccgcttcacgcggagggggatgagagggagatatccgtcgcggcggagatcctgcgaaaatccaggaagttcccctcagcgttgcccatatgggcacgcccggactacaatcctccactgctagagtcctggaaggacccggactacgtccctccggtggtacacggatgcccattgccacctaccaaggctcctccaataccacctccacggagaaagaggacggttgtcctgacagaatccaatgtgtcttctgccttggcggagctcgccactaagaccttcggtagctccggatcgtcggccgttgatagcggcacggcgaccgcccttcctgacctggcctccgacgacggtgacaaaggatccgacgttgagtcgtactcctccatgcccccccttgaaggggagccgggggaccccgatctcagcgacgggtcttggtctaccgtgagtgaggaggctagtgaggatgtcgtctgctgctcaatgtcctatacgtggacaggcgccctgatcacgccatgcgctgcggaggaaagtaagctgcccatcaacccgttgagcaactctttgctgcgtcaccacaacatggtctacgccacaacatcccgcagcgcaagcctccggcagaagaaggtcacctttgacagattgcaagtcctggatgatcattaccgggacgtactcaaggagatgaaggcgaaggcgtccacagttaaggctaagcttctatctatagaggaggcctgcaagctgacgcccccacattcggccaaatccaaatttggctatggggcaaaggacgtccggaacctatccagcagggccgttaaccacatccgctccgtgtgggaggacttgctggaagacactgaaacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggacctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 9 - DNA, 7979 bp, Artificial Sequence
  Thepolynucleotide sequence encodes sequences from HCV J4(B/RI) Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacggaggtggatggagtgcggttgcacaggtacgctccggcgtgcaaacctcttctacgggaggacgtcacgttccaggtcgggctcaaccaatacttggtcgggtcgcagctcccatgcgagcccgaaccggacgtaacagtgcttacttccatgctcaccgatccctcccacattacagcagagacggctaagcgtaggctggctagagggtctcccccctctttagccagctcatcagctatccagttgtctgcgccttctttgaaggcgacatgcactacccaccatgactccccggacgctgacctcatcgaggccaacctcttgtggcggcaggagatgggcggaaacatcactcgcgtggagtcagagaataaggtagtaattctggactctttcgaaccgcttcacgcggagggggatgagagggagatatccgtcgcggcggagatcctgcgaaaatccaggaagttcccctcagcgttgcccatatgggcacgcccggactacaatcctccactgctagagtcctggaaggacccggactacgtccctccggtggtacacggatgcccattgccacctaccaaggctcctccaataccacctccacggagaaagaggacggttgtcctgacagaatccaatgtgtcttctgccttggcggagctcgccactaagaccttcggtagctccggatcgtcggccgttgatagcggcacggcgaccgcccttcctgacctggcctccgacgacggtgacaaaggatccgacgttgagtcgtactcctccatgcccccccttgaaggggagccgggggaccccgatctcagcgacgggtcttggtctaccgtgagtgaggaggctagtgaggatgtcgtctgctgctcaatgtcctatacgtggacaggcgccctgatcacgccatgcgctgcggaggaaagtaagctgcccatcaacccgttgagcaactctttgctgcgtcaccacaacatggtctacgccacaacatcccgcagcgcaagcctccggcagaagaaggtcacctttgacagattgcaagtcctggatgatcattaccgggacgtactcaaggagatgaaggcgaaggcgtccacagttaaggctaagcttctatctatagaggaggcctgcaagctgacgcccccacattcggccaaatccaaatttggctatggggcaaaggacgtccggaacctatccagcagggccgttaaccacatccgctccgtgtgggaggacttgctggaagacactgaaacaccaattgacaccaccatcatggcaaaaagtgaggttttctgcgtccaaccagagaagggaggccgcaagccagctcgccttatcgtattcccagacctgggagttcgtgtatgcgagaagatggccctttacgacgtggtctccacccttcctcaggccgtgatgggctcctcatacggatttcaatactcccccaagcagcgggtcgagttcctggtgaatacctggaaatcaaagaaatgccctatgggcttctcatatgacacccgctgttttgactcaacggtcactgagagtgacattcgtgttgaggagtcaatttaccaatgttgtgacttggcccccgaggccagacaggccataaggtcgctcacagagcggctttacatcgggggtcccctgactaactcaaaagggcagaactgcggttatcgccggtgccgcgcaagtggcgtgctgacgactagctgcggtaataccctcacatgttacttgaaggccactgcagcctgtcgagctgcaaagctccaggactgcacgatgctcgtgaacggagacgaccttgtcgttatctgtgaaagcgcgggaacccaggaggatgcggcggccctacgagccttcacggaggctatgactaggtattccgccccccccggggatccgccccaaccagaatacgacctggagctgataacatcatgttcctccaatgtgtcagtcgcgcacgatgcatctggcaaaagggtatactacctcacccgtgaccccaccaccccccttgcacgggctgcgtgggagacagctagacacactccaatcaactcttggctaggcaatatcatcatgtatgcgcccaccctatgggcaaggatgattctgatgactcactttttctccatccttctagctcaagagcaacttgaaaaagccctggattgtcagatctacggggcttgctactccattgagccacttgacctacctcagatcattgaacgactccatggtcttagcgcatttacactccacagttactctccaggtgagatcaatagggtggcttcatgcctcaggaaacttggggtaccacccttgcgaacctggagacatcgggccagaagtgtccgcgctaagctactgtcccagggggggagggccgccacttgtggcagatacctctttaactgggcagtaaggaccaagcttaaactcactccaatcccggccgcgtcccagctggacttgtctggctggttcgtcgctggttacagcgggggagacatatatcacagcctgtctcgtgcccgaccccgctggtttccgttgtgcctactcctactttctgtaggggtaggcatttacctgctccccaaccgatgaacggggagctaaccactccaggccttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcaagt

SEQ ID NO: 10 - DNA, 7979 bp, Artificial Sequence
  Thepolynucleotide sequence encodes sequences from HCV J4(J4B/R1(C))Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacggaggtggatggagtgcggttgcacaggtacgctccggcgtgcaaacctcttctacgggaggacgtcacgttccaggtcgggctcaaccaatacttggtcgggtcgcagctcccatgcgagcccgaaccggacgtaacagtgcttacttccatgctcaccgatccctcccacattacagcagagacggctaagcgtaggctggctagagggtctcccccctctttagccagctcatcagctatccagttgtctgcgccttctttgaaggcgacatgcactacccaccatgactccccggacgctgacctcatcgaggccaacctcttgtggcggcaggagatgggcggaaacatcactcgcgtggagtcagagaataaggtagtaattctggactctttcgaaccgcttcacgcggagggggatgagagggagatatccgtcgcggcggagatcctgcgaaaatccaggaagttcccctcagcgttgcccatatgggcacgcccggactacaatcctccactgctagagtcctggaaggacccggactacgtccctccggtggtacacggatgcccattgccacctaccaaggctcctccaataccacctccacggagaaagaggacggttgtcctgacagaatccaatgtgtcttctgccttggcggagctcgccactaagaccttcggtagctccggatcgtcggccgttgatagcggcacggcgaccgcccttcctgacctggcctccgacgacggtgacaaaggatccgacgttgagtcgtactcctccatgcccccccttgaaggggagccgggggaccccgatctcagcgacgggtcttggtctaccgtgagtgaggaggctagtgaggatgtcgtctgctgctcaatgtcctatacgtggacaggcgccctgatcacgccatgcgctgcggaggaaagtaagctgcccatcaacccgttgagcaactctttgctgcgtcaccacaacatggtctacgccacaacatcccgcagcgcaagcctccggcagaagaaggtcacctttgacagattgcaagtcctggatgatcattaccgggacgtactcaaggagatgaaggcgaaggcgtccacagttaaggctaagcttctatctatagaggaggcctgcaagctgacgcccccacattcggccaaatccaaatttggctatggggcaaaggacgtccggaacctatccagcagggccgttaaccacatccgctccgtgtgggaggacttgctggaagacactgaaacaccaattgacaccaccatcatggcaaaaagtgaggttttctgcgtccaaccagagaagggaggccgcaagccagctcgccttatcgtattcccagacctgggagttcgtgtatgcgagaagatggccctttacgacgtggtctccacccttcctcaggccgtgatgggctcctcatacggatttcaatactcccccaagcagcgggtcgagttcctggtgaatacctggaaatcaaagaaatgccctatgggcttctcatatgacacccgctgttttgactcaacggtcactgagagtgacattcgtgttgaggagtcaatttaccaatgttgtgacttggcccccgaggccagacaggccataaggtcgctcacagagcggctttacatcgggggtcccctgactaactcaaaagggcagaactgcggttatcgccggtgccgcgcaagtggcgtgctgacgactagctgcggtaataccctcacatgttacttgaaggccactgcagcctgtcgagctgcaaagctccaggactgcacgatgctcgtgaacggagacgaccttgtcgttatctgtgaaagcgcgggaacccaggaggatgcggcggccctacgagccttcacggaggctatgactaggtattccgccccccccggggatccgccccaaccagaatacgacctggagctgataacatcatgttcctccaatgtgtcagtcgcgcacgatgcatctggcaaaagggtatactacctcacccgtgaccccaccaccccccttgcacgggctgcgtgggagacagctagacacactccaatcaactcttggctaggcaatatcatcatgtatgcgcccaccctatgggcaaggatgattctgatgactcactttttctccatccttctagctcaagagcaacttgaaaaagccctggattgtcagatctacggggcttgctactccattgagccacttgacctacctcagatcattgaacgactccatggtcttagcgcatttacactccacagttactctccaggtgagatcaatagggtggcttcatgcctcaggaaacttggggtaccacccttgcgaacctggagacatcgggccagaagtgtccgcgctaagctactgtcccagggggggagggccgccacttgtggcagatacctctttaactgggcagtaaggaccaagcttaaactcactccaatcccggccgcgtcccagctggacttgtctggctggttcgtcgctggttacagcgggggagacatatatcacagcctgtctcgtgcccgaccccgctggtttccgttgtgcctactcctactttctgtaggggtaggcatttacctgctccccaaccgatgaacggggagctaaccactccaggccttaagccatttcctgttttttttttttttttttttttttttttctttttttttttctttcctttccttctttttttcctttctttttcccttctttaatggtggctccatcttagccctagtcacggctagctgtgaaaggtccgtgagccgcttgactgcagagagtgctgatactggcctctctgcagatcaagt

SEQ ID NO: 11 - DNA, 7979 bp, Artificial Sequence
  Thepolynucleotide sequence encodes sequences from HCV J4 Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtagacctggacctcgtcggctggcaggcgccccccggggcgcgctccatgacaccatgcagctgtggcagctcggacctttacttggtcacgagacatgctgatgtcattccggtgcgccggcgaggcgacagcaggggaagtctactctcccccaggcccgtctcctacctgaaaggctcctcgggtggtccattgctttgcccttcggggcacgtcgtgggcgtcttccgggctgctgtgtgcacccggggggtcgcgaaggcggtggacttcatacccgttgagtctatggaaactaccatgcggtctccggtcttcacagacaactcaacccccccggctgtaccgcagacattccaagtggcacatctgcacgctcctactggcagcggcaagagcaccaaagtgccggctgcgtatgcagcccaagggtacaaggtgctcgtcctgaacccgtccgttgccgccaccttagggtttggggcgtatatgtccaaggcacacggtatcgaccctaacatcagaactggggtaaggaccattaccacgggcggctccattacgtactccacctatggcaagttccttgccgacggtggctgttctgggggcgcctatgacatcataatatgtgatgagtgccactcaactgactcgactaccatcttgggcatcggcacagtcctggaccaagcggagacggctggagcgcggctcgtcgtgctcgccaccgctacacctccgggatcggttaccgtgccacaccccaatatcgaggaaataggcctgtccaacaatggagagatccccttctatggcaaagccatccccattgaggccatcaagggggggaggcatctcattttctgccattccaagaagaaatgtgacgagctcgccgcaaagctgacaggcctcggactgaacgctgtagcatattaccggggccttgatgtgtccgtcataccgcctatcggagacgtcgttgtcgtggcaacagacgctctaatgacgggtttcaccggcgattttgactcagtgatcgactgcaatacatgtgtcacccagacagtcgacttcagcttggatcccaccttcaccattgagacgacgaccgtgccccaagacgcggtgtcgcgctcgcaacggcgaggtagaactggcaggggtaggagtggcatctacaggtttgtgactccaggagaacggccctcgggcatgttcgattcttcggtcctgtgtgagtgctatgacgcgggctgtgcttggtatgagctcacgcccgctgagacctcggttaggttgcgggcttacctaaatacaccagggttgcccgtctgccaggaccatctggagttctgggagagcgtcttcacaggcctcacccacatagatgcccacttcctgtcccagactaaacaggcaggagacaactttccttacctggtggcatatcaagctacagtgtgcgccagggctcaagctccacctccatcgtgggaccaaatgtggaagtgtctcatacggctgaaacctacactgcacgggccaacacccctgctgtataggctaggagccgtccaaaatgaggtcatcctcacacaccccataactaaatacatcatggcatgcatgtcggctgacctggaggtcgtcactagcacctgggtgctggtaggcggagtccttgcagctttggccgcatactgcctgacgacaggcagtgtggtcattgtgggcaggatcatcttgtccgggaagccagctgtcgttcccgacagggaagtcctctaccaggagttcgatgagatggaagagtgtgcctcacaacttccttacatcgagcagggaatgcagctcgccgagcaattcaagcaaaaggcgctcgggttgttgcaaacggccaccaagcaagcggaggctgctgctcccgtggtggagtccaagtggcgagcccttgagaccttctgggcgaagcacatgtggaatttcatcagcggaatacagtacctagcaggcttatccactctgcctggaaaccccgcgatagcatcattgatggcatttacagcttctatcactagcccgctcaccacccaaaacaccctcctgtttaacatcttggggggatgggtggctgcccaactcgctcctcccagcgctgcgtcagctttcgtgggcgccggcatcgccggagcggctgttggcagcataggccttgggaaggtgctcgtggacatcttggcgggctatggggcaggggtagccggcgcactcgtggcctttaaggtcatgagcggcgaggtgccctccaccgaggacctggtcaacttactccctgccatcctctctcctggtgccctggtcgtcggggtcgtgtgcgcagcaatactgcgtcggcacgtgggcccgggagagggggctgtgcagtggatgaaccggctgatagcgttcgcttcgcggggtaaccacgtctcccctacgcactatgtgcctgagagcgacgctgcagcacgtgtcactcagatcctctctagccttaccatcactcaactgctgaagcggctccaccagtggattaatgaggactgctctacgccatgctccggctcgtggctaagggatgtttgggattggatatgcacggtgttgactgacttcaagacctggctccagtccaaactcctgccgcggttaccgggagtccctttcctgtcatgccaacgcgggtacaagggagtctggcggggggacggcatcatgcaaaccacctgcccatgcggagcacagatcgccggacatgtcaaaaacggttccatgaggatcgtagggcctagaacctgcagcaacacgtggcacggaacgttccccatcaacgcatacaccacgggaccttgcacaccctccccggcgcccaactattccagggcgctatggcgggtggctgctgaggagtacgtggaggttacgcgtgtgggggatttccactacgtgacgggcatgaccactgacaacgtaaagtgcccatgccaggttccggcccccgaattcttcacggaggtggatggagtgcggttgcacaggtacgctccggcgtgcaaacctcttctacgggaggacgtcacgttccaggtcgggctcaaccaatacttggtcgggtcgcagctcccatgcgagcccgaaccggacgtaacagtgcttacttccatgctcaccgatccctcccacattacagcagagacggctaagcgtaggctggctagagggtctcccccctctttagccagctcatcagctatccagttgtctgcgccttctttgaaggcgacatgcactacccaccatgactccccggacgctgacctcatcgaggccaacctcttgtggcggcaggagatgggcggaaacatcactcgcgtggagtcagagaataaggtagtaattctggactctttcgaaccgcttcacgcggagggggatgagagggagatatccgtcgcggcggagatcctgcgaaaatccaggaagttcccctcagcgttgcccatatgggcacgcccggactacaatcctccactgctagagtcctggaaggacccggactacgtccctccggtggtacacggatgcccattgccacctaccaaggctcctccaataccacctccacggagaaagaggacggttgtcctgacagaatccaatgtgtcttctgccttggcggagctcgccactaagaccttcggtagctccggatcgtcggccgttgatagcggcacggcgaccgcccttcctgacctggcctccgacgacggtgacaaaggatccgacgttgagtcgtactcctccatgcccccccttgaaggggagccgggggaccccgatctcagcgacgggtcttggtctaccgtgagtgaggaggctagtgaggatgtcgtctgctgctcaatgtcctatacgtggacaggcgccctgatcacgccatgcgctgcggaggaaagtaagctgcccatcaacccgttgagcaactctttgctgcgtcaccacaacatggtctacgccacaacatcccgcagcgcaagcctccggcagaagaaggtcacctttgacagattgcaagtcctggatgatcattaccgggacgtactcaaggagatgaaggcgaaggcgtccacagttaaggctaagcttctatctatagaggaggcctgcaagctgacgcccccacattcggccaaatccaaatttggctatggggcaaaggacgtccggaacctatccagcagggccgttaaccacatccgctccgtgtgggaggacttgctggaagacactgaaacaccaattgacaccaccatcatggcaaaaagtgaggttttctgcgtccaaccagagaagggaggccgcaagccagctcgccttatcgtattcccagacctgggagttcgtgtatgcgagaagatggccctttacgacgtggtctccacccttcctcaggccgtgatgggctcctcatacggatttcaatactcccccaagcagcgggtcgagttcctggtgaatacctggaaatcaaagaaatgccctatgggcttctcatatgacacccgctgttttgactcaacggtcactgagagtgacattcgtgttgaggagtcaatttaccaatgttgtgacttggcccccgaggccagacaggccataaggtcgctcacagagcggctttacatcgggggtcccctgactaactcaaaagggcagaactgcggttatcgccggtgccgcgcaagtggcgtgctgacgactagctgcggtaataccctcacatgttacttgaaggccactgcagcctgtcgagctgcaaagctccaggactgcacgatgctcgtgaacggagacgaccttgtcgttatctgtgaaagcgcgggaacccaggaggatgcggcggccctacgagccttcacggaggctatgactaggtattccgccccccccggggatccgccccaaccagaatacgacctggagctgataacatcatgttcctccaatgtgtcagtcgcgcacgatgcatctggcaaaagggtatactacctcacccgtgaccccaccaccccccttgcacgggctgcgtgggagacagctagacacactccaatcaactcttggctaggcaatatcatcatgtatgcgcccaccctatgggcaaggatgattctgatgactcactttttctccatccttctagctcaagagcaacttgaaaaagccctggattgtcagatctacggggcttgctactccattgagccacttgacctacctcagatcattgaacgactccatggtcttagcgcatttacactccacagttactctccaggtgagatcaatagggtggcttcatgcctcaggaaacttggggtaccacccttgcgaacctggagacatcgggccagaagtgtccgcgctaagctactgtcccagggggggagggccgccacttgtggcagatacctctttaactgggcagtaaggaccaagcttaaactcactccaatcccggccgcgtcccagctggacttgtctggctggttcgtcgctggttacagcgggggagacatatatcacagcctgtctcgtgcccgaccccgctggtttccgttgtgcctactcctactttctgtaggggtaggcatttacctgctccccaaccgatgaacggggagctaaccactccaggccttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcaagt

SEQ ID NO: 12 - DNA, 7979 bp, Artificial Sequence
  Thepolynucleotide sequence encodes sequences from HCV J4 Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtagacctggacctcgtcggctggcaggcgccccccggggcgcgctccatgacaccatgcagctgtggcagctcggacctttacttggtcacgagacatgctgatgtcattccggtgcgccggcgaggcgacagcaggggaagtctactctcccccaggcccgtctcctacctgaaaggctcctcgggtggtccattgctttgcccttcggggcacgtcgtgggcgtcttccgggctgctgtgtgcacccggggggtcgcgaaggcggtggacttcatacccgttgagtctatggaaactaccatgcggtctccggtcttcacagacaactcaacccccccggctgtaccgcagacattccaagtggcacatctgcacgctcctactggcagcggcaagagcaccaaagtgccggctgcgtatgcagcccaagggtacaaggtgctcgtcctgaacccgtccgttgccgccaccttagggtttggggcgtatatgtccaaggcacacggtatcgaccctaacatcagaactggggtaaggaccattaccacgggcggctccattacgtactccacctatggcaagttccttgccgacggtggctgttctgggggcgcctatgacatcataatatgtgatgagtgccactcaactgactcgactaccatcttgggcatcggcacagtcctggaccaagcggagacggctggagcgcggctcgtcgtgctcgccaccgctacacctccgggatcggttaccgtgccacaccccaatatcgaggaaataggcctgtccaacaatggagagatccccttctatggcaaagccatccccattgaggccatcaagggggggaggcatctcattttctgccattccaagaagaaatgtgacgagctcgccgcaaagctgacaggcctcggactgaacgctgtagcatattaccggggccttgatgtgtccgtcataccgcctatcggagacgtcgttgtcgtggcaacagacgctctaatgacgggtttcaccggcgattttgactcagtgatcgactgcaatacatgtgtcacccagacagtcgacttcagcttggatcccaccttcaccattgagacgacgaccgtgccccaagacgcggtgtcgcgctcgcaacggcgaggtagaactggcaggggtaggagtggcatctacaggtttgtgactccaggagaacggccctcgggcatgttcgattcttcggtcctgtgtgagtgctatgacgcgggctgtgcttggtatgagctcacgcccgctgagacctcggttaggttgcgggcttacctaaatacaccagggttgcccgtctgccaggaccatctggagttctgggagagcgtcttcacaggcctcacccacatagatgcccacttcctgtcccagactaaacaggcaggagacaactttccttacctggtggcatatcaagctacagtgtgcgccagggctcaagctccacctccatcgtgggaccaaatgtggaagtgtctcatacggctgaaacctacactgcacgggccaacacccctgctgtataggctaggagccgtccaaaatgaggtcatcctcacacaccccataactaaatacatcatggcatgcatgtcggctgacctggaggtcgtcactagcacctgggtgctggtaggcggagtccttgcagctttggccgcatactgcctgacgacaggcagtgtggtcattgtgggcaggatcatcttgtccgggaagccagctgtcgttcccgacagggaagtcctctaccaggagttcgatgagatggaagagtgtgcctcacaacttccttacatcgagcagggaatgcagctcgccgagcaattcaagcaaaaggcgctcgggttgttgcaaacggccaccaagcaagcggaggctgctgctcccgtggtggagtccaagtggcgagcccttgagaccttctgggcgaagcacatgtggaatttcatcagcggaatacagtacctagcaggcttatccactctgcctggaaaccccgcgatagcatcattgatggcatttacagcttctatcactagcccgctcaccacccaaaacaccctcctgtttaacatcttggggggatgggtggctgcccaactcgctcctcccagcgctgcgtcagctttcgtgggcgccggcatcgccggagcggctgttggcagcataggccttgggaaggtgctcgtggacatcttggcgggctatggggcaggggtagccggcgcactcgtggcctttaaggtcatgagcggcgaggtgccctccaccgaggacctggtcaacttactccctgccatcctctctcctggtgccctggtcgtcggggtcgtgtgcgcagcaatactgcgtcggcacgtgggcccgggagagggggctgtgcagtggatgaaccggctgatagcgttcgcttcgcggggtaaccacgtctcccctacgcactatgtgcctgagagcgacgctgcagcacgtgtcactcagatcctctctagccttaccatcactcaactgctgaagcggctccaccagtggattaatgaggactgctctacgccatgctccggctcgtggctaagggatgtttgggattggatatgcacggtgttgactgacttcaagacctggctccagtccaaactcctgccgcggttaccgggagtccctttcctgtcatgccaacgcgggtacaagggagtctggcggggggacggcatcatgcaaaccacctgcccatgcggagcacagatcgccggacatgtcaaaaacggttccatgaggatcgtagggcctagaacctgcagcaacacgtggcacggaacgttccccatcaacgcatacaccacgggaccttgcacaccctccccggcgcccaactattccagggcgctatggcgggtggctgctgaggagtacgtggaggttacgcgtgtgggggatttccactacgtgacgggcatgaccactgacaacgtaaagtgcccatgccaggttccggcccccgaattcttcacggaggtggatggagtgcggttgcacaggtacgctccggcgtgcaaacctcttctacgggaggacgtcacgttccaggtcgggctcaaccaatacttggtcgggtcgcagctcccatgcgagcccgaaccggacgtaacagtgcttacttccatgctcaccgatccctcccacattacagcagagacggctaagcgtaggctggctagagggtctcccccctctttagccagctcatcagctatccagttgtctgcgccttctttgaaggcgacatgcactacccaccatgactccccggacgctgacctcatcgaggccaacctcttgtggcggcaggagatgggcggaaacatcactcgcgtggagtcagagaataaggtagtaattctggactctttcgaaccgcttcacgcggagggggatgagagggagatatccgtcgcggcggagatcctgcgaaaatccaggaagttcccctcagcgttgcccatatgggcacgcccggactacaatcctccactgctagagtcctggaaggacccggactacgtccctccggtggtacacggatgcccattgccacctaccaaggctcctccaataccacctccacggagaaagaggacggttgtcctgacagaatccaatgtgtcttctgccttggcggagctcgccactaagaccttcggtagctccggatcgtcggccgttgatagcggcacggcgaccgcccttcctgacctggcctccgacgacggtgacaaaggatccgacgttgagtcgtactcctccatgcccccccttgaaggggagccgggggaccccgatctcagcgacgggtcttggtctaccgtgagtgaggaggctagtgaggatgtcgtctgctgctcaatgtcctatacgtggacaggcgccctgatcacgccatgcgctgcggaggaaagtaagctgcccatcaacccgttgagcaactctttgctgcgtcaccacaacatggtctacgccacaacatcccgcagcgcaagcctccggcagaagaaggtcacctttgacagattgcaagtcctggatgatcattaccgggacgtactcaaggagatgaaggcgaaggcgtccacagttaaggctaagcttctatctatagaggaggcctgcaagctgacgcccccacattcggccaaatccaaatttggctatggggcaaaggacgtccggaacctatccagcagggccgttaaccacatccgctccgtgtgggaggacttgctggaagacactgaaacaccaattgacaccaccatcatggcaaaaagtgaggttttctgcgtccaaccagagaagggaggccgcaagccagctcgccttatcgtattcccagacctgggagttcgtgtatgcgagaagatggccctttacgacgtggtctccacccttcctcaggccgtgatgggctcctcatacggatttcaatactcccccaagcagcgggtcgagttcctggtgaatacctggaaatcaaagaaatgccctatgggcttctcatatgacacccgctgttttgactcaacggtcactgagagtgacattcgtgttgaggagtcaatttaccaatgttgtgacttggcccccgaggccagacaggccataaggtcgctcacagagcggctttacatcgggggtcccctgactaactcaaaagggcagaactgcggttatcgccggtgccgcgcaagtggcgtgctgacgactagctgcggtaataccctcacatgttacttgaaggccactgcagcctgtcgagctgcaaagctccaggactgcacgatgctcgtgaacggagacgaccttgtcgttatctgtgaaagcgcgggaacccaggaggatgcggcggccctacgagccttcacggaggctatgactaggtattccgccccccccggggatccgccccaaccagaatacgacctggagctgataacatcatgttcctccaatgtgtcagtcgcgcacgatgcatctggcaaaagggtatactacctcacccgtgaccccaccaccccccttgcacgggctgcgtgggagacagctagacacactccaatcaactcttggctaggcaatatcatcatgtatgcgcccaccctatgggcaaggatgattctgatgactcactttttctccatccttctagctcaagagcaacttgaaaaagccctggattgtcagatctacggggcttgctactccattgagccacttgacctacctcagatcattgaacgactccatggtcttagcgcatttacactccacagttactctccaggtgagatcaatagggtggcttcatgcctcaggaaacttggggtaccacccttgcgaacctggagacatcgggccagaagtgtccgcgctaagctactgtcccagggggggagggccgccacttgtggcagatacctctttaactgggcagtaaggaccaagcttaaactcactccaatcccggccgcgtcccagctggacttgtctggctggttcgtcgctggttacagcgggggagacatatatcacagcctgtctcgtgcccgaccccgctggtttccgttgtgcctactcctactttctgtaggggtaggcatttacctgctccccaaccgatgaacggggagctaaccactccaggccttaagccatttcctgttttttttttttttttttttttttttttctttttttttttctttcctttccttctttttttcctttctttttcccttctttaatggtggctccatcttagccctagtcacggctagctgtgaaaggtccgtgagccgcttgactgcagagagtgctgatactggcctctctgcagatcaagt

SEQ ID NO: 13 - DNA, 7989 bp, Artificial Sequence
  Thepolynucleotide sequence encodes sequences from HCV J4(BB7/J4NS5B)Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctatccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaagtaagctgcccatcaacccgttgagcaactctttgctgcgtcaccacaacatggtctacgccacaacatcccgcagcgcaagcctccggcagaagaaggtcacctttgacagattgcaagtcctggatgatcattaccgggacgtactcaaggagatgaaggcgaaggcgtccacagttaaggctaagcttctatctatagaggaggcctgcaagctgacgcccccacattcggccaaatccaaatttggctatggggcaaaggacgtccggaacctatccagcagggccgttaaccacatccgctccgtgtgggaggacttgctggaagacactgaaacaccaattgacaccaccatcatggcaaaaagtgaggttttctgcgtccaaccagagaagggaggccgcaagccagctcgccttatcgtattcccagacctgggagttcgtgtatgcgagaagatggccctttacgacgtggtctccacccttcctcaggccgtgatgggctcctcatacggatttcaatactcccccaagcagcgggtcgagttcctggtgaatacctggaaatcaaagaaatgccctatgggcttctcatatgacacccgctgttttgactcaacggtcactgagagtgacattcgtgttgaggagtcaatttaccaatgttgtgacttggcccccgaggccagacaggccataaggtcgctcacagagcggctttacatcgggggtcccctgactaactcaaaagggcagaactgcggttatcgccggtgccgcgcaagtggcgtgctgacgactagctgcggtaataccctcacatgttacttgaaggccactgcagcctgtcgagctgcaaagctccaggactgcacgatgctcgtgaacggagacgaccttgtcgttatctgtgaaagcgcgggaacccaggaggatgcggcggccctacgagccttcacggaggctatgactaggtattccgccccccccggggatccgccccaaccagaatacgacctggagctgataacatcatgttcctccaatgtgtcagtcgcgcacgatgcatctggcaaaagggtatactacctcacccgtgaccccaccaccccccttgcacgggctgcgtgggagacagctagacacactccaatcaactcttggctaggcaatatcatcatgtatgcgcccaccctatgggcaaggatgattctgatgactcactttttctccatccttctagctcaagagcaacttgaaaaagccctggattgtcagatctacggggcttgctactccattgagccacttgacctacctcagatcattgaacgactccatggtcttagcgcatttacactccacagttactctccaggtgagatcaatagggtggcttcatgcctcaggaaacttggggtaccacccttgcgaacctggagacatcgggccagaagtgtccgcgctaagctactgtcccagggggggagggccgccacttgtggcagatacctctttaactgggcagtaaggaccaagcttaaactcactccaatcccggccgcgtcccagctggacttgtctggctggttcgtcgctggttacagcgggggagacatatatcacagcctgtctcgtgcccgaccccgctggtttccgttgtgcctactcctactttctgtaggggtaggcatttacctgctccccaaccgatgaacgggtacgtaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 14 - DNA, 7989 bp, Artificial Sequence
  Thepolynucleotide sequence encodes sequences from HCV H77(pBB7-SN)Replicons
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctatccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacgggtacgtaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 15 - DNA, 22 bp, Artificial Sequence
  The sequence encodes DNA fragments amplified by using pHCV rep1b(BB7) as template and the primer pairs
cgtctgctgctcgatgtcctac

SEQ ID NO: 16 - DNA, 45 bp, Artificial Sequence
  The sequence encodes DNA fragments amplified by using pHCV rep1b(BB7) as template and the primer pairs
ctcccccaaccgatgaacgggtacgtaaacactccaggccaatag

SEQ ID NO: 17 - DNA, 27 bp, Artificial Sequence
  The sequence encodes DNA fragments amplified by using pHCV rep1b(BB7) as template and the primer pairs
gcactagtacttgatctgcagagaggc

SEQ ID NO: 18 - DNA, 45 bp, Artificial Sequence
  The sequence encodes DNA fragments amplified by using pHCV rep1b(BB7) as template and the primer pairs
ctattggcctggagtgtttacgtacccgttcatcggttgggggag

SEQ ID NO: 19 - DNA, 23 bp, Artificial Sequence
  To generate pBB7/H77NS5B, the HCV type 1a H77 NS5B gene was first amplified by using H77 DNA and primers
cctggacaggcgcactgatcacc

SEQ ID NO: 20 - DNA, 22 bp, Artificial Sequence
  To generate this sequence, two DNA fragments were amplified by using pHCVrep1b(BB7) as template and the primer pairs
gaggacttgctggaagacactg

SEQ ID NO: 21 - DNA, 26 bp, Artificial Sequence
  To generate this sequence, two DNA fragments were amplified by using pHCVrep1b(BB7) as template and the primer pairs
caggagtacttgatctgcagagaggc

SEQ ID NO: 22 - DNA, 40 bp, Artificial Sequence
  To generate this sequence, two DNA fragments were amplified by using pHCVrep1b(BB7) as template and the primer pairs
ctttagccagctcatcagctatccagttgtctgcgccttc

SEQ ID NO: 23 - DNA, 40 bp, Artificial Sequence
  To generate this sequence, two DNA fragments were amplified by using pHCVrep1b(BB7) as template and the primer pairs
gaaggcgcagacaactggatagctgatgagctggctaaac

SEQ ID NO: 24 - DNA, 24 bp, Artificial Sequence
  To generate this sequence, the neoomycin resitance gene was performed using PCR
tcaagaccgacctgtccggtgccc

SEQ ID NO: 25 - DNA, 24 bp, Artificial Sequence
  To generate this sequence, the neoomycin resitance gene was performed using PCR
cttgagcctggcgaacagttcggc

SEQ ID NO: 26 - DNA, 20 bp, Artificial Sequence
  To generate this sequence, the neoomycin resitance gene was performed using PCR
accacagtccatgccatcac

SEQ ID NO: 27 - DNA, 20 bp, Artificial Sequence
  To generate this sequence, the neoomycin resitance gene was performed using PCR
tccaccaccctgttgctgta

SEQ ID NO: 28 - DNA, 18 bp, Artificial Sequence
  The total cellular was isolated using RNeasy Mini Kit (Qiagen)
ccggctacctgcccattc

SEQ ID NO: 29 - DNA, 21 bp, Artificial Sequence
  The total cellular was isolated using RNeasy Mini Kit (Qiagen)
ccagatcatccgatcgacaag

SEQ ID NO: 30 - DNA, 24 bp, Artificial Sequence
  The total cellular was isolated using RNeasy Mini Kit (Qiagen)
acatcgcatcgagcgagcacgtac

SEQ ID NO: 31 - DNA, 25 bp, Artificial Sequence
  DNA primer sequences used in making the chimeric replicon constructs. Primers of the invention was derived from plasmids.
catccagatgtacaccaatgtggac

SEQ ID NO: 32 - DNA, 26 bp, Artificial Sequence
  DNA primer sequences used in making the chimeric replicon constructs. Primers of the invention was derived from plasmids.
catcgcccgaattcttcacagaattg

SEQ ID NO: 33 - DNA, 26 bp, Artificial Sequence
  DNA primer sequences used in making the chimeric replicon constructs. Primers of the invention was derived from plasmids.
caattctgtgaagaattcgggcgatg

SEQ ID NO: 34 - DNA, 24 bp, Artificial Sequence
  DNA primer sequences used in making the chimeric replicon constructs. Primers of the invention was derived from plasmids.
gtaacaccaattgacactaccatc

SEQ ID NO: 35 - DNA, 24 bp, Artificial Sequence
  DNA primer sequences used in making the chimeric replicon constructs. Primers of the invention was derived from plasmids.
gatggtagtgtctattggtgttac

SEQ ID NO: 36 - DNA, 56 bp, Artificial Sequence
  DNA primer sequences used in making the chimeric replicon constructs. Primers of the invention was derived from plasmids.
gcactagtacttgatctgcagagaggccagtatcagcactctctgcagtcaagcgg

SEQ ID NO: 37 - DNA, 40 bp, Artificial Sequence; identical to SEQ ID NO: 22
  DNA primer sequences used in making the chimeric replicon constructs. Primers of the invention was derived from plasmids.

SEQ ID NO: 38 - DNA, 40 bp, Artificial Sequence; identical to SEQ ID NO: 23
  DNA primer sequences used in making the chimeric replicon constructs. Primers of the invention was derived from plasmids.

SEQ ID NO: 39 - DNA, 45 bp, Artificial Sequence
  DNA primer sequences used in making the chimeric replicon constructs. Primers of the invention was derived from plasmids.
gagatggagcggacagctggatagccgaggagctggccatagaag

SEQ ID NO: 40 - DNA, 45 bp, Artificial Sequence
  DNA primer sequences used in making the chimeric replicon constructs. Primers of the invention was derived from plasmids.
cttctatggccagctcctcggctatccagctgtccgctccatctc

SEQ ID NO: 41 - DNA, 22 bp, Artificial Sequence
  DNA primer sequences used in making the chimeric replicon constructs. Primers of the invention was derived from plasmids.
cgtctgctgctcgatgtcctac

SEQ ID NO: 42 - DNA, 45 bp, Artificial Sequence; identical to SEQ ID NO: 16
  DNA primer sequences used in making the chimeric replicon constructs. Primers of the invention was derived from plasmids.

SEQ ID NO: 43 - DNA, 23 bp, Artificial Sequence
  DNA primer sequences used in making the chimeric replicon constructs. Primers of the invention was derived from plasmids.
cctggacaggcgcactgatcacc

SEQ ID NO: 44 - DNA, 22 bp, Artificial Sequence
  DNA primer sequences used in making the chimeric replicon constructs.  Primers of the invention was derived from plasmids.
gaggacttgctggaagacactg

SEQ ID NO: 45 - DNA, 26 bp, Artificial Sequence
  DNA primer sequences used in making the chimeric replicon constructs.  Primers of the invention was derived from plasmids.
caggagtacttgatctgcagagaggc

SEQ ID NO: 46 - DNA, 27 bp, Artificial Sequence
  DNA primer sequences used in making the chimeric replicon constructs.  Primers of the invention was derived from plasmids.
gcactagtacttgatctgcagagaggc

SEQ ID NO: 47 - DNA, 45 bp, Artificial Sequence; identical to SEQ ID NO: 18
  DNA primer sequences used in making the chimeric replicon constructs.  Primers of the invention was derived from plasmids.

SEQ ID NO: 48 - DNA, 24 bp, Artificial Sequence
  DNA primer sequences used in making the chimeric replicon constructs. Primers of the invention was derived from plasmids.
tcaagaccgacctgtccggtgccc

SEQ ID NO: 49 - DNA, 24 bp, Artificial Sequence
  DNA primer sequences used in making the chimeric replicon constructs. Primers of the invention was derived from plasmids.
cttgagcctggcgaacagttcggc

SEQ ID NO: 50 - DNA, 20 bp, Artificial Sequence
  DNA primer sequences used in making the chimeric replicon constructs. Primers of the invention was derived from plasmids.
accacagtccatgccatcac

SEQ ID NO: 51 - DNA, 20 bp, Artificial Sequence
  DNA primer sequences used in making the chimeric replicon constructs. Primers of the invention was derived from plasmids.
tccaccaccctgttgctgta

SEQ ID NO: 52 - DNA, 18 bp, Artificial Sequence
  DNA primer sequences used in making the chimeric replicon constructs. Primers of the invention was derived from plasmids.
ccggctacctgcccattc

SEQ ID NO: 53 - DNA, 21 bp, Artificial Sequence
  DNA primer sequences used in making the chimeric replicon constructs. Primers of the invention was derived from plasmids.
ccagatcatccgatcgacaag

SEQ ID NO: 54 - DNA, 24 bp, Artificial Sequence
  DNA primer sequences used in making the chimeric replicon constructs. Primers of the invention was derived from plasmids.
acatcgcatcgagcgagcacgtac